(19) United States
(12) Patent Application Publication
(10) Pub. No.: US 2007/0010875 A1
(43) Pub. Date: Jan. 11, 2007

(54) Method and apparatus to attach an unsupported surgical component

(76) Inventors: Hugh H. Trout, Chevy Chase, MD (US); Frank V. Patterson, Exeter, NH (US); Howard M. Tanner, Naperville, IL (US); Ronald J. Ehmsen, Goleta, CA (US)

Correspondence Address: KELLEY DRYE & WARREN LLP, 3050 K STREET, NW, SUITE 400, WASHINGTON, DC, 20007, US

(21) Appl. No.: 11/366,604

(22) Filed: May 2, 2006

Related U.S. Application Data
(63) Continuation-in-part of application No. 10/389,972, filed on Mar. 18, 2003, now abandoned.
(63) Continuation-in-part of application No. 10/678,622, filed on Oct. 3, 2003, now abandoned.
Application No. 10/678,622 is a division of application No. 09/108,191, filed on Jul. 1, 1998, now Pat. No. 6,635,066.
Application No. 09/108,191 is a division of application No. 08/896,415, filed on Jul. 18, 1997, now Pat. No. 5,944,750.
(60) Provisional application No. 60/364,601, filed on Mar. 18, 2002.
(60) Provisional application No. 60/382,084, filed on May 22, 2002.
(60) Provisional application No. 60/374,833, filed on Apr. 24, 2002.
(60) Provisional application No. 60/375,807, filed on Apr. 29, 2002.
(60) Provisional application No. 60/051,209, filed on Jun. 30, 1997.

(51) Int. Cl. A61F 2/06

(52) U.S. Cl. 623/1.36

(57) ABSTRACT

An insertion apparatus comprises an introducer sheath, a surgical component, at least two attachment wires, and a longitudinal wire. The insertion apparatus is used for deploying a surgical component to a site in a vessel. The introducer sheath has a lumen, a proximal opening and a distal opening with an appropriate diameter and length allowing fro insertion and navigation through the vessel. The surgical component has a tube portion with a top and at least one limb. The surgical component corresponds to the site in the vessel and fits within the lumen of the introducer sheath. The attachment wires have a first and a second end. The first end is releasably connected to the top of the surgical component and the second end converges above the top of the surgical component. The activation of a releasing mechanism releases the first end from the top of the surgical component. The longitudinal wire anchors from the second end extending through the surgical component and through the introducer sheath to the releasing mechanism outside the vessel.

### CROSS-REFERENCE TO RELATED APPLICATIONS

The present invention is a continuation-in-part application of U.S. patent application Ser. No. 10/389,972 filed Mar. 18, 2003 which relates to, and is entitled to the benefit of the earlier filing date and priority of U.S. Application No. 60/364,601 filed Mar. 18, 2002, U.S. Application No. 60/382,084, filed May 22, 2002; U.S. Application No. 60/374,833 filed Apr. 24, 2002; and U.S. Application No. 60/375,807 filed Apr. 29, 2002. The present application is also a continuation-in-part application of U.S. patent application Ser. No. 10/678,622 filed Oct. 20, 2003 which relates to is a divisional application of utility application Ser. No. 09/108,191, filed on Jul. 1, 1988, which is a divisional application of application Ser. No. 08/896,415, filed Jul. 18, 1997, which claims the benefit of provisional Application No. 06/051,209, filed on Jun. 30, 1997. The entire content of all aforementioned applications is hereby incorporated by reference.

### FIELD OF THE INVENTION

The present invention relates to apparatus and methods for performing a surgical procedure. More particularly, the present invention relates to apparatus and methods to attach an unsupported surgical component.

### BACKGROUND OF THE INVENTION

An aneurysm is a ballooning of the wall of an artery resulting from the weakening of the artery due to disease or other conditions. Left untreated, the aneurysm will frequently rupture, resulting in loss of blood through the rupture and death.

Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in that portion of the abdominal aorta between the renal arteries and the iliac arteries. This portion of the abdominal aorta is particularly susceptible to weakening, resulting in an aortic aneurysm. Such an aneurysm is often located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm because no statistical benefit exists in performing such procedures.

Aneurysms in the abdominal aorta are associated with a particularly high mortality rate; accordingly, current medical standards call for urgent operative repair. Abdominal surgery, however, results in substantial stress to the body. Although the mortality rate for an aortic aneurysm is high, there is also considerable mortality and morbidity associated with open surgical intervention to repair an aortic aneurysm. This intervention involves penetrating the abdominal wall to the location of the aneurysm to reinforce or replace the diseased section of the aortic aneurysm. A prosthetic device, typically a synthetic tube graft, is used for this purpose. The graft serves to exclude the aneurysm from the circulatory system, thus relieving pressure and stress on the weakened section of the aorta at the aneurysm.

Repair of an aortic aneurysm by surgical means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of intra-abdominal surgery. It is, therefore, desirable to reduce the mortality and morbidity associated with intra-abdominal surgical intervention.

In recent years, methods have been developed to attempt to treat an aortic aneurysm without the attendant risks of intra-abdominal surgical intervention. Among them are inventions disclosed and claimed in Kornberg, U.S. Pat. No. 4,562,596 for Aortic Graft, Device and Method for Performing an Intraluminal Abdominal Aortic Aneurysm Repair; Lazarus, U.S. Pat. No. 4,787,899 for Intraluminal Graft Device, System and Method; and Taheri, U.S. Pat. No. 5,042,707 for Intravascular Stapler, and Method of Operating Same.

Although in recent years certain techniques have been developed that may reduce the stress, morbidity, and risk of mortality associated with surgical intervention to repair aortic aneurysms, none of the systems that have been developed effectively treat the aneurysm and exclude the affected section of aorta from the pressures and stresses associated with circulation. None of the devices disclosed in the references provide a reliable and quick means to reinforce an aneurysmal artery. In addition, all of the prior references require a sufficiently large section of healthy aorta surrounding the aneurysm to ensure attachment of the graft. The neck of the aorta at the cephalad end (i.e., above the aneurysm) is usually sufficient to maintain a graft's attachment means. However, when an aneurysm is located near the iliac arteries, there may be an ill-defined neck or no neck below the aneurysm. Such an ill-defined neck would have an insufficient amount of healthy aortic tissue to which to successfully mount a graft. Furthermore, much of the abdominal aorta wall may be calcified which may make it extremely difficult to attach the graft to the wall. Furthermore, the prior art does not disclose surgical devices that can be used during a surgical procedure that address these concerns. Others have developed devices that are not easily manipulated or oriented during intraluminal surgical procedures.

It is therefore an advantage of some, but not necessarily all, embodiments of the present invention to provide an improved apparatus and method to attach an unsupported surgical component.

Additional advantages of various embodiments of the invention are set forth, in part, in the description that follows and, in part, will be apparent to one of ordinary skill in the art from the description and/or from the practice of the invention.

### SUMMARY OF THE INVENTION

Responsive to the foregoing challenges, Applicant has developed an innovative method and apparatus to attach an unsupported surgical component.

An embodiment of the present invention is an insertion apparatus for deploying a surgical component to a site in a vessel comprising: attachment means for attaching the surgical component to the insertion apparatus, wherein the attachment means is releasably connected to the surgical component; and release means for releasing the attachment means from the surgical component, wherein the release means is in communication with the attachment means.

An embodiment of the present invention is an insertion apparatus for deploying a surgical component to a site in a vessel comprising: at least one attachment wire with a first end and a second end; a support wire; wherein the first end of the at least one attachment wire is releaseably connected to the surgical component and the second end is connected to the support wire; and wherein the attachment wire and support wire positions the surgical component at a site in the vessel.

An embodiment of the present invention is an insertion apparatus for deploying a surgical component to a site in a vessel comprising: an attachment hoop releasably connected to the surgical component; at least one attachment wire with a first end and a second end; a support wire; wherein the first end of the at least one attachment wire is connected to the attachment hoop and the second end of the at least one attachment wire is connected to the support wire; and wherein the insertion apparatus positions the surgical component in the vessel.

An embodiment of the present invention is an insertion system for deploying a surgical component to a site in a vessel comprising: a surgical component; an insertion apparatus; attachment means for attaching the surgical component to the insertion apparatus, wherein the attachment means is releasably connected to the surgical component; and release means for releasing the attachment means from the surgical component, wherein the release means is in communication with the attachment means.

An embodiment of the present invention is a method for positioning a surgical component to a site in a vessel comprising the steps of: introducing an insertion apparatus proximal to the site in the vessel; activating the insertion apparatus; and withdrawing the insertion apparatus. An embodiment of the present invention further comprises the step of fastening the surgical component to the vessel.

In accordance with an embodiment of the present invention, there is provided an introducer sheath for deploying a surgical component to a site in a vessel. The apparatus includes an introducer sheath, a surgical component, at least two attachment wires, and a longitudinal wire. The introducer sheath has a lumen, a proximal opening and a distal opening with an appropriate diameter and length allowing for insertion and navigation through the vessel. The surgical component has a tube portion with a top and at least one limb. The surgical component corresponds to the site in the vessel and fits within the lumen of the introducer sheath. The attachment wires have a first and a second end. The first end is releasably connected to the top of the surgical component and the second end converges above the top of the surgical component. The activation of a releasing mechanism releases the first end from the top of the surgical component. The longitudinal wire anchors from the second end extending through the surgical component and through the introducer sheath to the releasing mechanism outside the vessel.

In accordance with an embodiment of the present invention, there is provided a method for attaching a surgical component with an insertion apparatus to site in a vessel comprising the steps of: inserting the insertion apparatus to the site in the vessel; positioning the surgical component to the site in the vessel; fastening the surgical component to the vessel; activating a release mechanism of the insertion apparatus; and withdrawing the insertion apparatus.

It is to be understood that both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention and, together with the detailed description, serve to explain the principles of the present invention.

### BRIEF DESCRIPTION OF THE DRAWINGS

In order to assist the understanding of this invention, reference will now be made to the appended drawings, in which like reference characters refer to like elements. The drawings are exemplary only, and should not be construed as limiting the invention.

FIGS. 1 and 2 are perspective views of a surgical component with and without distinctive markings according to an embodiment of the present invention.

FIG. 3 is a perspective view of a surgical component according to an embodiment of the present invention.

FIGS. 4, 5, and 6 are enlarged perspective views of embodiments of a release mechanism according to embodiments of the present invention.

FIG. 7 is a perspective view of an embodiment of the attachment wires secured to a bulb with a subsequent device attached thereto.

FIG. 8 is a perspective view of an expanded surgical component with an insulated longitudinal wire according to an embodiment of the present invention.

FIG. 9 is a perspective view of a contracted surgical component according to an embodiment of the present invention.

FIG. 10 is a perspective view of an embodiment of the surgical component attached to a release mechanism employing an electrical current to separate the attachment wires.

FIG. 11 is a perspective view of an embodiment of the surgical component having distinctive markings and sutures attached to the ends of the limbs.

FIGS. 12 and 13 are perspective views of the graft packed with a short sheath, which may be flared according to an embodiment of the present invention.

FIGS. 14, 15, and 16 are perspective views of the graft with limbs trimmed to an appropriate length by the inserter to accommodate patient size according to an embodiment of the present invention.

FIGS. 17 and 18 are perspective views of the introducer sheath with a detachable hemostatic valve and a dilator according to an embodiment of the present invention.

FIG. 19 is a perspective view of introducer sheath and the short sheath wherein the short sheath containing the graft is introduced into the introducer sheath according to an embodiment of the present invention.

FIG. 20 is a perspective view of the graft advanced to the area just before the soft area of the introducer sheath where the graft is entirely removed from the short sheath according to an embodiment of the present invention.

FIGS. 21 and 22 are perspective views of the graft with the short sheath removed and the expansion of the soft or resilient area according to an embodiment of the present invention.

FIG. 23 is a perspective view of the graft advanced to the end of the introducer sheath according to an embodiment of the present invention.

FIGS. 24 and 25 are perspective views of a long delivery catheter and a short sheath with a graft according to an embodiment of the present invention.

FIGS. 26 and 27 are perspective views of the top of the short sheath being placed at the opening of the long delivery catheter according to an embodiment of the present invention.

FIG. 28 is a perspective view of the entire graft assembly advanced into the long delivery catheter according to an embodiment of the present invention.

FIG. 29 is a perspective view of an embodiment of the insertion apparatus within an aneurysm.

FIGS. 30 and 31 are perspective views of an embodiment of the insertion apparatus with a snare catheter within an aneurysm.

FIGS. 32 and 33 are perspective views of the introducer sheath pulled back from the graft in the aneurysm according to an embodiment of the present invention.

FIG. 34 is a perspective view of the unsupported graft within the aortic neck with the attachment wires unrestrained according to an embodiment of the present invention.

FIG. 35 is a perspective view of the snare catheter passed through the left femoral artery out to the right femoral artery according to an embodiment of the present invention.

FIGS. 36 and 37 are perspective views of the aneurysm with graft limbs in the right femoral artery and the left femoral artery according to an embodiment of the present invention.

FIG. 38 is a perspective view of a fastener delivery catheter inserted and positioned within the graft located in the aortic neck according to an embodiment of the present invention.

FIG. 39 is a perspective view of the graft with fasteners inserted around the top portion of the graft according to an embodiment of the present invention.

FIGS. 40 and 41 are perspective views releasing the attachment wires from a graft with the fasteners inserted according to an embodiment of the present invention.

FIG. 42 is a perspective view of the snare catheter inserted above the attachment wires according to an embodiment of the present invention.

FIGS. 43 and 44 are perspective views of the snare catheter tightened around the attachment wires for removal according to an embodiment of the present invention.

FIG. 45 is a perspective view of the graft in the aneurysm without the attachment wires according to an embodiment of the present invention.

FIG. 46 is a perspective view of two stents inserted in the right and left limbs of the graft according to an embodiment of the present invention.

FIG. 47 is a perspective view of the sutures attached to the right and left limbs of the graft which have been cut by a suture cutter according to an embodiment of the present invention.

FIG. 48 is a perspective view of an embodiment of the longitudinal wire with a containment sheath according to an embodiment of the present invention.

FIG. 49 is a perspective view of the containment sheath enclosing the attachment wires of the graft according to an embodiment of the present invention.

FIG. 50 is a perspective view of an alternate embodiment of the short sheath with an open or partial sheath attached according to an embodiment of the present invention.

FIG. 51 is a perspective view of an insertion sheath with a short sheath according to an embodiment of the present invention.

FIG. 52 is a perspective view of an alternate embodiment of the short sheath with a closed circumferential sheath according to an embodiment of the present invention.

FIGS. 53 and 54 are perspective views of the graft when a release mechanism is applied according to an embodiment of the present invention.

FIGS. 55 and 56 are perspective views of the constraining and releasing of the attachment wires according to an embodiment of the present invention.

FIG. 57 is a perspective view of a method to attach and detach the tube portion of the graft from the attachment wires according to an embodiment of the present invention.

FIG. 58 is a perspective view of the tube portion of a graft contained within the circumferential short sheath according to an embodiment of the present invention.

FIG. 59 is a perspective view of the introducer sheath having a plurality of soft resilient areas and a hemostatic valve according to an embodiment of the present invention.

FIG. 60 is a perspective view of introducer sheaths having a variety of connectors and hemostatic valves according to an embodiment of the present invention.

FIG. 61 is a perspective view of multiple ports within the inner wall of the introducer sheath according to an embodiment of the present invention.

FIG. 62 is a cross section view of the introducer sheath with a passageway according to an embodiment of the present invention.

FIG. 63 is a perspective view of an introducer sheath containing a plurality of soft spots attached by a connector to a double port also having a plurality of soft spots according to an embodiment of the present invention.

FIG. 64 is a perspective view of the introducer sheath having a user-controlled variable restricting device according to an embodiment of the present invention.

FIGS. 65 and 66 are perspective views of a suture cutter with a suture according to an embodiment of the present invention.

FIG. 67 is a perspective view of an embodiment of a closed circumferential sheath with a partial or open sheath according to an embodiment of the present invention.

FIG. 68A is a perspective view of a prosthetic bifurcated tube graft and bifurcated cuff according to a preferred embodiment of the present invention;

FIG. 68B is a perspective view of a prosthetic bifurcated tube graft and bifurcated cuff according to another embodiment of the present invention;

FIG. 69A is a perspective view of the prosthetic bifurcated tube graft and bifurcated cuff of FIG. 68A secured within the abdominal aorta;

FIG. 69B is a perspective view of the prosthetic bifurcated tube graft and bifurcated cuff of FIG. 68B secured within the abdominal aorta;

FIG. 70 is a perspective view of a prosthetic tube graft and cuff according to another embodiment of the present invention;

FIG. 71 is a perspective view of the prosthetic tube graft and cuff of FIG. 70 secured within the abdominal aorta;

FIG. 72 is a perspective view of the connection between the prosthetic tube graft and the cuff;

FIG. 73 is a side view of the prosthetic tube graft of FIG. 71 secured to a secondary cuff;

FIG. 74 is an exploded view of the connection between the prosthetic tube graft and secondary cuff as shown in FIG. 73;

FIG. 75 is a perspective view of attachment cuffs according to another embodiment of the present invention;

FIG. 76 is a perspective view of the flexible attachment cuff according to embodiments of the present invention;

FIG. 77 is a perspective view of the attachment cuffs of FIG. 75 having a prosthetic tube graft secured between the attachment cuffs;

FIG. 78 is a perspective view of an IntraVascular Endoscopy (IVE) based repair system according to an embodiment of the present invention containing an embodiment of a visualization device according to the present invention;

FIG. 79 is an end view of the IntraVascular Endoscopy (IVE) based repair system according to the embodiment of FIG. 78;

FIG. 80 is an end view of the visualization device depicted in FIG. 78;

FIG. 81 is another perspective view of the IntraVascular Endoscopy (IVE) based repair system illustrating the guide wire and articulation cables exiting the housing of the repair system;

FIG. 82A is a perspective view of an IntraVascular Endoscopy (IVE) based repair system according to an embodiment of the present invention containing an embodiment of a penetration device according to the present invention and an embodiment of a fastener cartridge according to the present invention;

FIG. 82B is a perspective view of an IVE based repair system according to another embodiment of the present invention containing a penetration device and fastener cartridge according to the present invention;

FIG. 82C is a perspective view of an IVE based repair system according to the embodiment of FIG. 82B containing a penetration device and fastener cartridge according to another embodiment of the present invention;

FIG. 82D is a perspective view of an IVE based repair system according to another embodiment of the present invention containing a penetration device and fastener cartridge according to the present invention;

FIG. 83 is an end view of the penetration device according to an embodiment of the present invention;

FIG. 84 is an end view of the penetration device according to another embodiment of the present invention;

FIG. 85 is an end view of the fastener cartridge according to the embodiment of FIG. 82

FIG. 86 is a perspective view of an advancing mechanism of a penetration device according to an embodiment of the present invention;

FIG. 87 is a schematic view of another advancing mechanism of a penetration device and fastener cartridge according to another embodiment of the present invention;

FIGS. 88 and 89 are perspective views of an IntraVascular UltraSound (IVUS) based repair apparatus according to another embodiment of the present invention containing a visualization device and a penetration device;

FIG. 90 is a cross sectional view of a housing according to an embodiment of the present invention;

FIG. 91 is an end view of a penetration device depicted in FIG. 88;

FIGS. 92 and 93 are perspective views of a wire fastener for securing the cuff detail of a surgical cuff to a vessel wall according to an embodiment of the present invention;

FIGS. 94 and 95 are perspective views of a wire fastener according to another embodiment of the present invention for securing the cuff detail of a surgical cuff to a vessel wall;

FIGS. 96 and 97 are perspective views of a wire fastener according to another embodiment of the present invention for securing the cuff detail of a surgical cuff to a vessel wall;

FIGS. 98, 99a, 99b, 99c, 99d and 99e are perspective views of a fastener according to another embodiment of the present invention for securing the cuff to a vessel wall;

FIG. 100 is a schematic view of an embodiment of the penetration device according to the present invention having fasteners, as shown in FIGS. 98, 99a, 99b and 99c stored thereon;

FIG. 101 is a schematic view of an another embodiment of the penetration device according to the present invention having fasteners, as shown in FIGS. 98, 99a, 99b and 99c stored therein;

FIGS. 102 and 103 are perspective views illustrating the fastener attachment of the cuff detail to the vessel wall using a fastener as shown in FIGS. 96 and 97 according to an embodiment of the present invention;

FIG. 104 is a perspective view of another embodiment of an IntraVascular Endoscopy (IVE) based repair system according to an another embodiment of the present invention;

FIG. 105 is a perspective view on an introducer sheath device according to the present invention; and,

FIG. 106 is a cross sectional view of a seal assembly for the introducer sheath device according to an embodiment of the present invention.

### DETAILED DESCRIPTION OF EMBODIMENTS OF THE INVENTION

Reference will now be made in detail to embodiments of the present invention, examples of which are illustrated in the accompanying drawings.

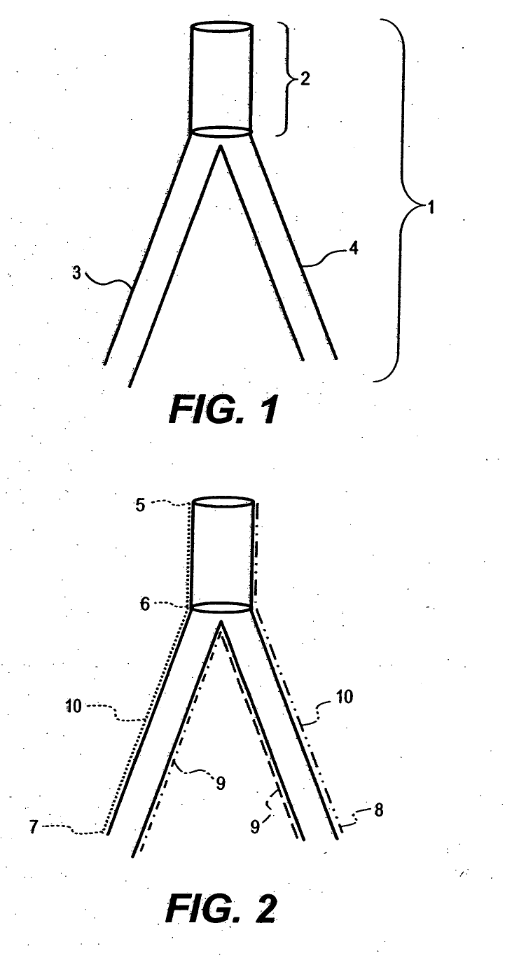

The following methods and apparatus may be used with any suitable surgical components. By way of example only, the following paragraphs describe methods of use and the apparatus with surgical components, such as, but not limited to, grafts, prosthetic grafts, endografts, patches, or any other suitable surgical component. For purposes of explanation only, these surgical components may be on-the-shelf ready for use or custom fabricated. The surgical components may have the tube portion 2, as shown in FIG. 1, with fixed length. The surgical component may be a patch or tubular in shape, and may or may not have limbs. If a custom made surgical component were required, it is likely to be in an unusual circumstance and that would be known in advance (because of the preoperative three dimensional (3D) computerized tomography (CT) scan or any other appropriate scan or modality) and a special order could be placed. The following examples are explanatory only and not meant to be limiting of the type of surgical component that may be used.

FIG. 1 depicts a surgical component 1, which may be, for purposes of example only, and is not limited to, a prosthetic bifurcated graft 1. This surgical component may have a fixed length tube portion 2 of any suitable size and may be, but is not limited to, from about 1-10 cm and may be comprised of, but is not limited to, Dacron, polyester, or PTFE. Graft limbs 3 and 4 may be supplied along with the intent that they will be trimmed to fit the patient size.

Depending on the size of the vessel in the patient, each tube diameter may be supplied with three different limb diameters. For example, the middle diameter may be one half of the tube diameter and the two others may be about 2 mm less and about 2 mm more than half the diameter of the tube portion. Thus, a 20 mm tube may come in three versions: 1) 20 mm tube and 8 mm limb diameter; 2) 20 mm tube and 10 mm limb diameter; and 3) 20 mm tube and 12 mm limb diameter. It is also possible to have one limb diameter larger than the other.

In FIG. 2, distinctive markings depicted by rings, dots and dashes may be added to the graft. Distinctive markings may be comprised of radiopaque markers or any other suitable distinctive markings, radiopaque or not, to facilitate easy identification under imaging techniques, including, but not limited to, fluoroscopy, magnetic resonance (MR), or any other suitable imaging modality. Radiopaque markers with surgical components are described in U.S. application Ser. No. 10/173,028 filed Jun. 18, 2001, the entire disclosure and subject matter of which is hereby incorporated herein by reference. These markers may be small pieces of radiopaque material or may be vaporized radiopaque material imbedded into the graft material, or of any other suitable material and affixed by any other suitable method. These markers will allow the inserter to locate the top 5, the bifurcation 6, and the ends of the limbs 7 and 8. These markers will also enable the inserter to distinguish among the medial 9 and lateral 10 portions of the graft limbs.

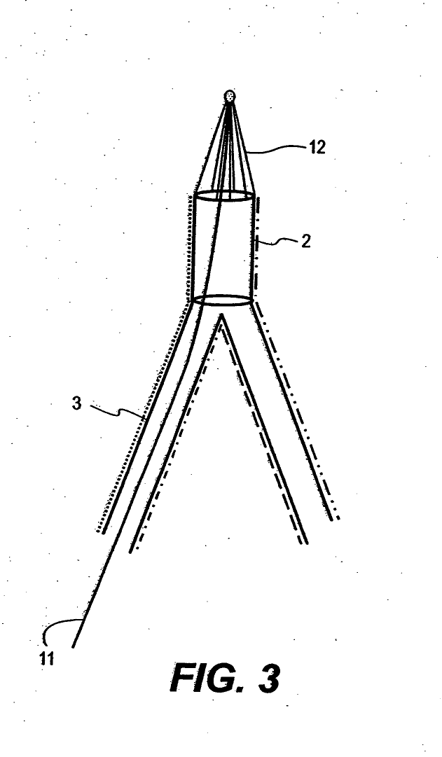

In FIG. 3, attachment means may comprise a support wire 11, attachment wire 12, and may further comprise additional support structures, including, but not limited to, at least one ring or hoop structure. Support wire 11 may have a slight bend or a tip deflection capability and may be inserted through the left 4 or right 3 limb of the graft 1 up through the tube portion 2 of the graft 1. The support wire 11 may be insulated. In FIGS. 3-6, a plurality of attachment wires 12 are connected to the graft 1 and to the support wire 11, either directly or through an intermediate structure, such as, but not limited to, a ring or hoop, described in more detail below. The attachment wires 12 may be connected to the top 5 of the graft 1 shown in FIG. 2.

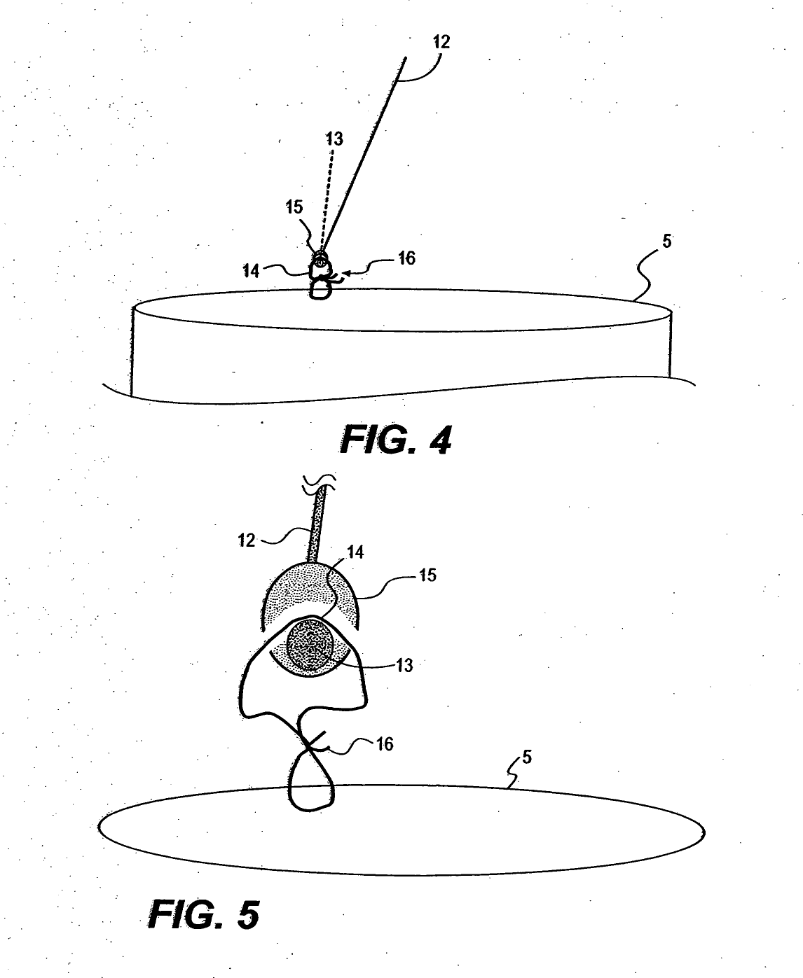

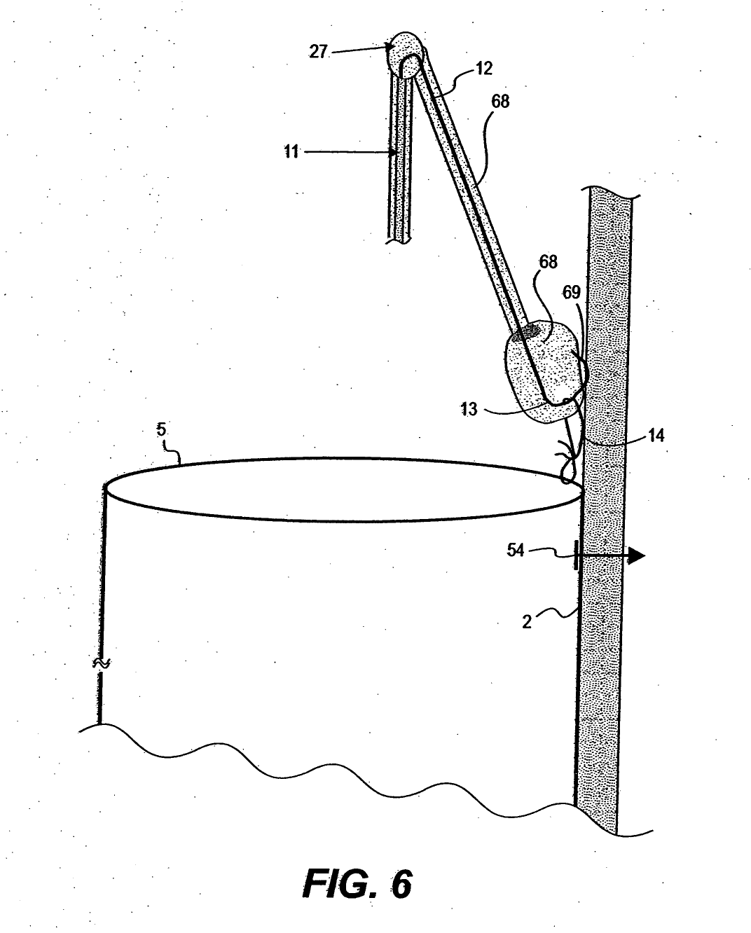

Details of the attachment wires 12 are shown in three blow-up drawings, FIGS. 4, 5, and 6. In one embodiment, an electric current is applied to the attachment wires 12. Attachment wires 12 connect to a filament 13 and/or a ball 15 that increases in temperature when a current is supplied. The filament 13 heats and burns through the suture 14 releasing the attachment wires from the graft 3. Release means may comprise an electrical current, heat, vibration, laser, dissolvable sutures, adhesive, metal alloys, or any other suitable release mechanism. In the embodiment shown in FIGS. 4 and 5, the suture 14 is cut by the hot filament 13 and separates from the ball 15. Because of the method used to tie the sutures, suture fragments are discouraged from embolizing elsewhere. FIG. 6 depicts another embodiment of the release mechanism where the attachment wires 12 are insulated with any suitable material 68. When a current is applied to the insulated 68 attachment wires 12, the filament 13 burns and severs the suture 14 attached to the top 5 of the graft 1. An exposed portion of the filament 13 may be used to ground the wire through tissue, such as the aortic wall. Multiple different circuit designs may be selected. For instance, instead of using a patient ground (electrical return path), the ground can be provided with a separate conductor onboard the device. Additionally, a feedback circuit, or any other suitable means, may be added to indicate to the user when the separation had been successfully completed.

A knot 16 of the suture 14 may be placed above the graft to decrease the bulkiness of the graft especially as the graft is being pushed/pulled through the introducer sheath 95. The balls 15 located near the top 5 of the graft 1 can be at different levels in order to reduce the bulk in any one transverse plane. Moreover, this method of attachment/release can be used in holding and detaching any foreign material in any vessel, artery or vein or in any tissue where a suture is a temporary attachment mechanism. A similar release mechanism could be achieved with magnets, glues that dissolve upon electrical or heat stimulation, or metal alloys that separate in response to heat or electrical current, or any other suitable method.

It will be apparent to those skilled in the art that variations and modifications of the present invention can be made without departing from the scope or spirit of the invention. For instance, as the need to insert objects into body orifices (whether percutaneously, through small incisions, or endoscopically) increases, it is progressively more important to reduce the size of these objects during their insertion as much as possible. One way to accomplish this objective is to insert components of the object through a small opening into the area where they will be attached or deployed. Once the components are inserted they may then be attached to each other to form a larger object. This may be accomplished via a scope or sheath inserted into an open orifice, such as, but not limited to, the trachea, the esophagus, the anus, the nose, the vagina or the urethra. Likewise, it may be done through an incision or skin penetration into the chest, abdomen, soft tissue, or a joint. It also may be accomplished through an incision or percutaneous penetration into an artery or vein remote from the desired site of implantation.

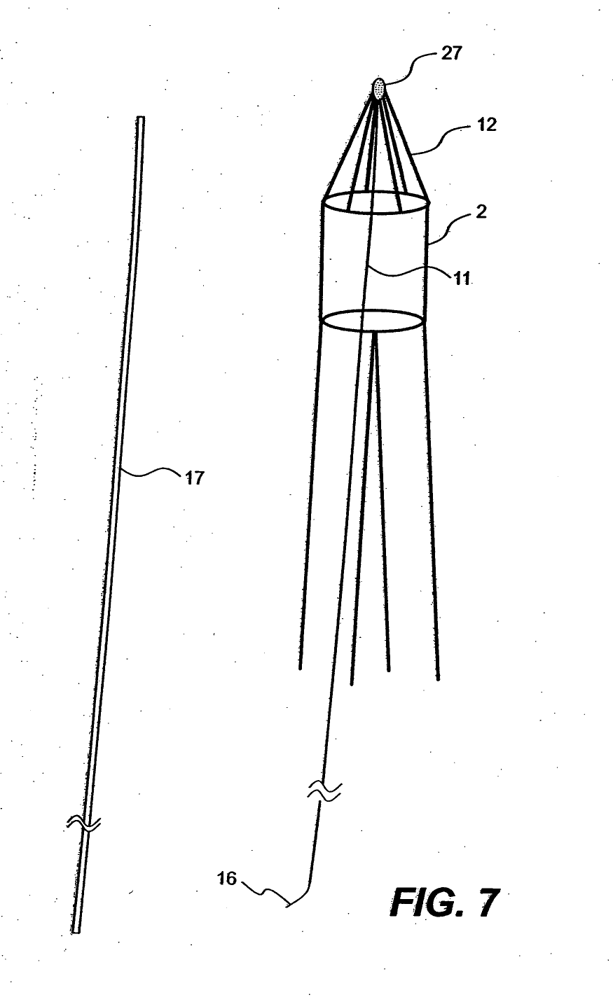

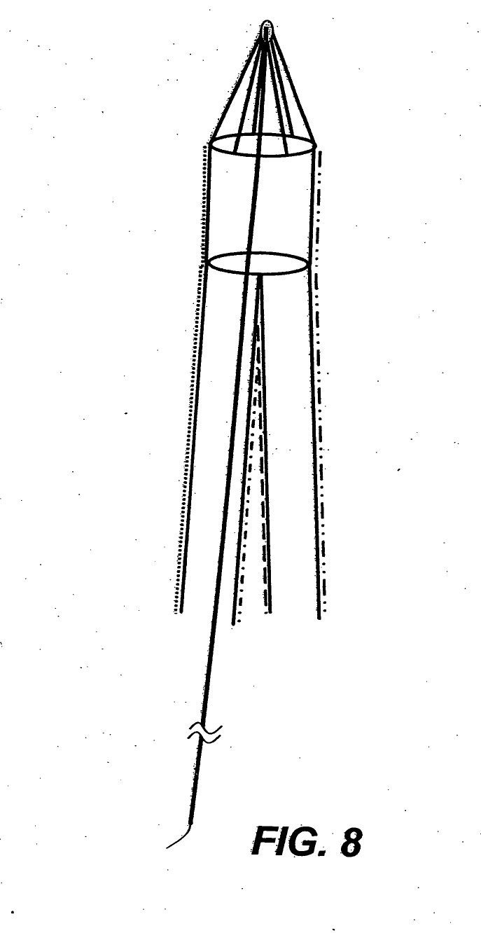

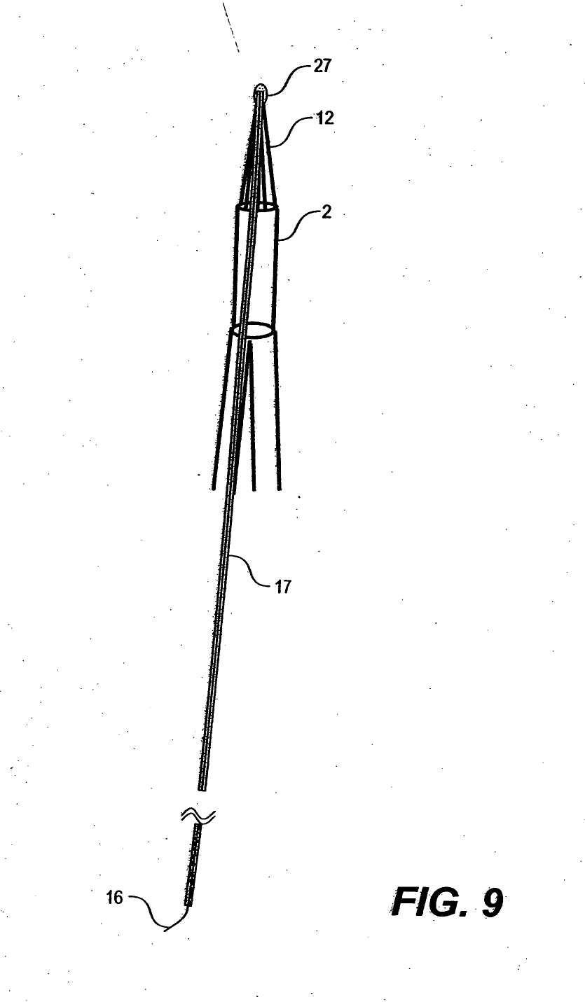

As an example of such an approach, an embodiment shown in FIG. 7, a very fine support wire 11, which may be, but is not limited to, a wire, a flexible suture, or any other suitable object is attached to a bulb 27 to which attachment wires 12 are connected on one and, on the other, to a graft 1 or the tube portion 2, such as, but not limited to, a prosthetic graft. This entire device may be compressed and delivered into an aorta through a small catheter. The wire 11 may have a smaller diameter so as to reduce the size of the device as it is being inserted. It may be necessary, however, for the device to be manipulated in such a way that the smaller diameter wire 11 is not capable of facilitating. In such an instance, it may be desirable to introduce over, about, or adjacent to the wire 11 at the distal end 16 a subsequent device 17 that attaches to a part of the bulb 27 so as to provide capabilities not provided by the small diameter wire 11. For instance, the subsequent device 17 may provide more support for the device, insulation for the wire 68 as in FIG. 8, or some sort of manipulating system that allows torsion or positioning control or control of the angulation of the attachment wires 12 from an expanded configuration in FIG. 7 to a contracted configuration in FIG. 9, or for any other suitable purpose. The subsequent device 17 may be attached to the support wire 11 and/or the bulb 27 by any suitable mechanism for attachment, such as, but not limited to, a snap-on mechanism, magnet, male/female type connector, screw-in attachment, or twist-lock mechanism. Further, the wire 11 with the subsequent device 17 may be disassembled, as desired, by any suitable release mechanism, such as, but not limited to, a switch, lever, heat, magnet, current applied, or current located to a control handle.

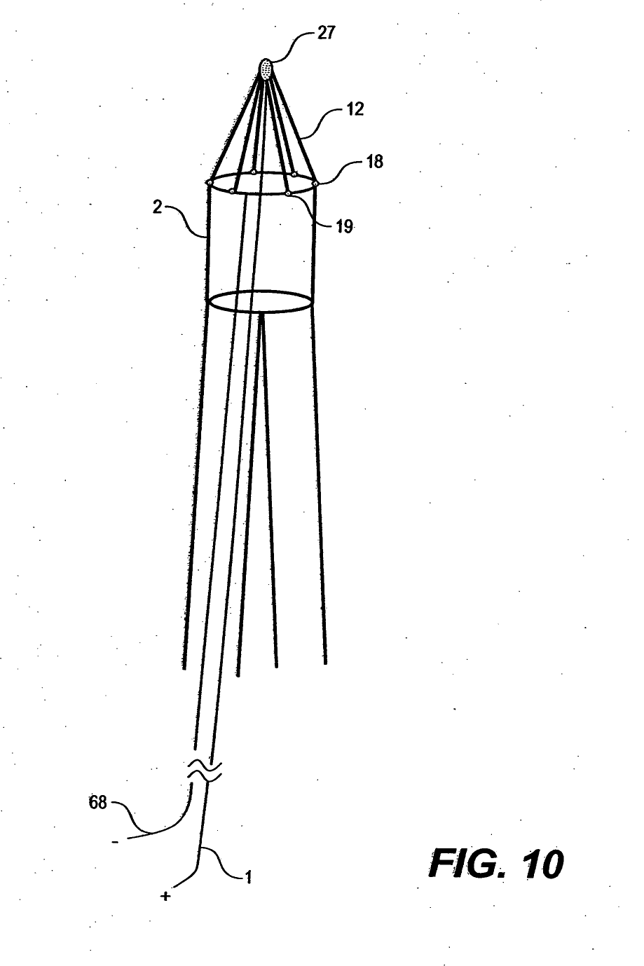

In addition, FIG. 10 depicts in another embodiment a mechanism to separate the attachment wires 12 from the prosthetic graft 1. An electrical current is applied to insulated 68 wires 11. In an embodiment, a high resistance connection 18 may then separate the attachment wires 12 from a thin wire or suture 19 disposed at or near the graft 1. For example, the connector 18 may be a metal alloy that weakens/separates when subjected to a current or it may be a filament that heats and severs an adjacent suture or some other such connector element that allows separation when a current is applied. FIG. 10 depicts a bipolar approach, but a monopolar mechanism with the ground being an adjacent arterial wall is also considered within the scope of the present invention. Thus, it is intended that the present invention cover all such modifications and variations of the invention, provided they come within the scope of the appended claims and their equivalents.

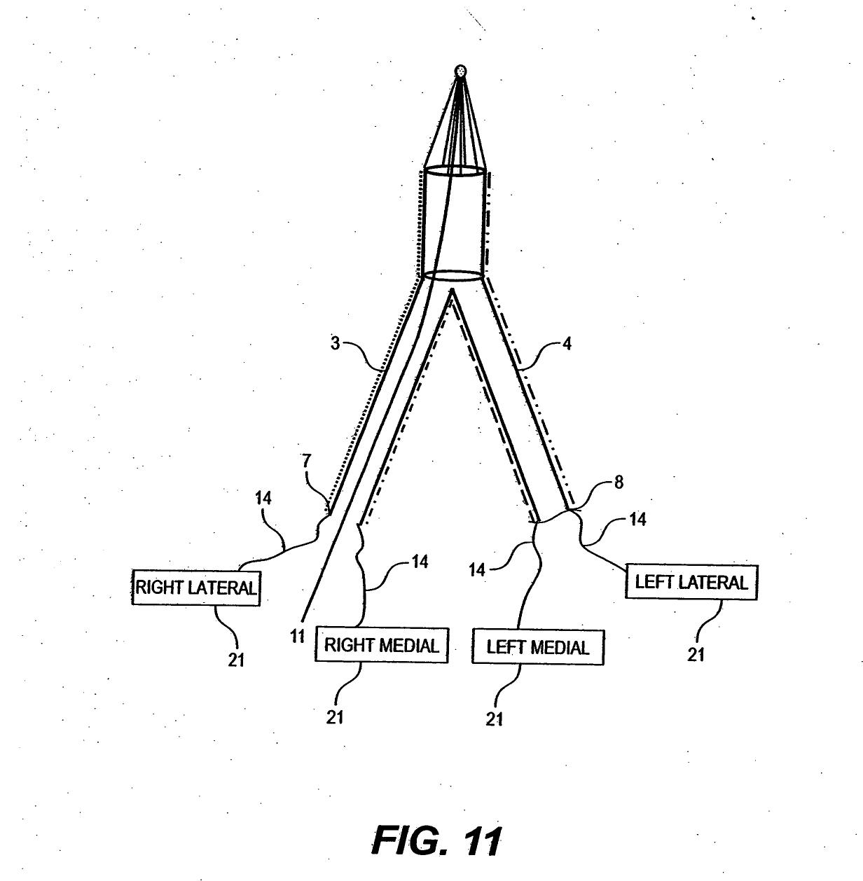

As shown in FIG. 11, a further embodiment includes a plurality of sutures 14 added to the end portions of the limbs 7 and 8. The sutures 14 may be attached to identification tags 21. The sutures 14 may also be removed when the limbs are trimmed to the appropriate length, at which time they may be replaced. The sutures 14 may be included in the packaged graft in order to provide a certainty as to the identification of the right 3 and left 4 limbs as well as the medial 9 and lateral 10 portions of the limb.

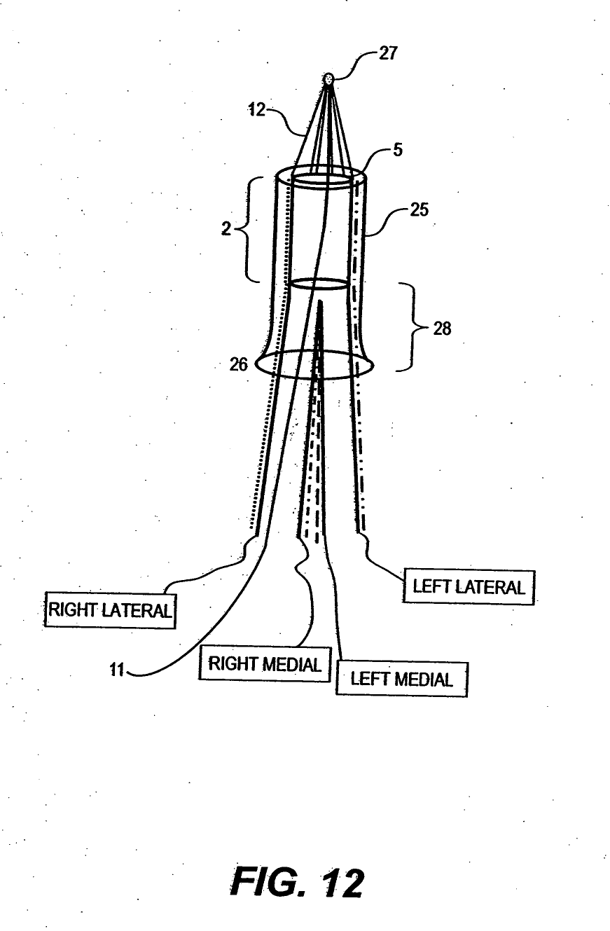

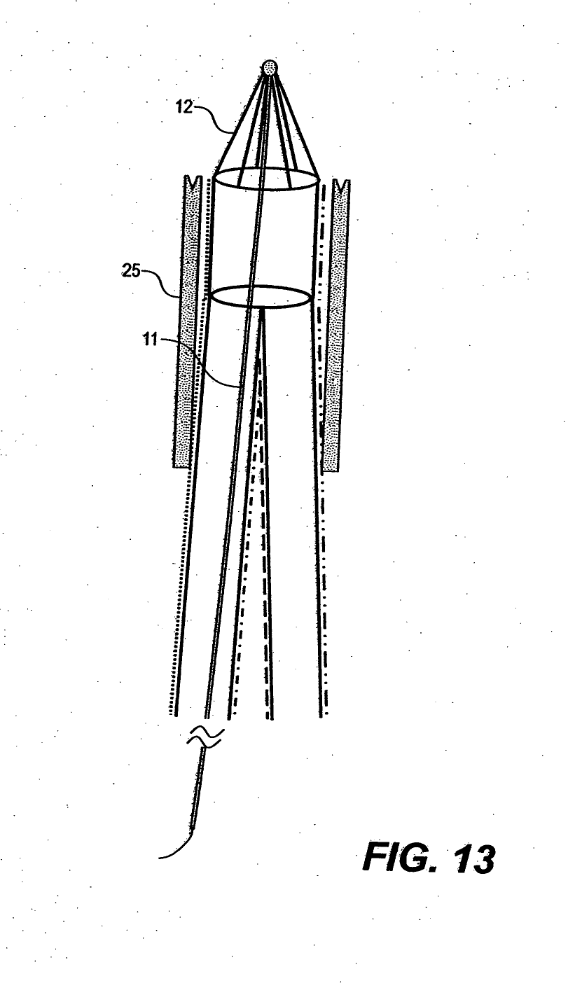

As depicted in FIGS. 12 and 13, the graft 1 may be packed in a short sheath 25. In an embodiment, the short sheath 25 may be flared at the bottom 26, as shown in FIG. 12. In FIGS. 11, 12, and 13, the support wire 11 may be attached to the attachment wires 12 at bulb 27. In FIG. 5, the attachment wires 12 may be attached to the filament balls 15 that, in turn, are attached to sutures 14 that, in turn, are attached to the top 5 of the tube portion of the graft 1. This arrangement allows for the bulk of the bulb 27 of the support wire 11, the attachment wires 12, the filament ball 15 in FIG. 5 and the knots 16 in FIG. 5 to be above the top of the graft as the graft is placed within one or more sheaths. The ball 15 is located at or near the tube portion 2 of the graft 1 whereas the bulb 27 is located above the tube portion 2 securing the attachment wires 12. The ball or bulb 15 or 27 can be the same structural element or may be different to accommodate the release mechanism. In an embodiment, the tube portion 2 may be a fixed length, such as, but is not limited to, about 2.5 cm and the upper portion of the limbs 28 included in the short sheath 25 may be, but is not limited to, about 3 cm. The tube portion 2 and the upper portion of the limbs 28 included in the short sheath 25 may be any suitable size depending upon the surgical procedure.

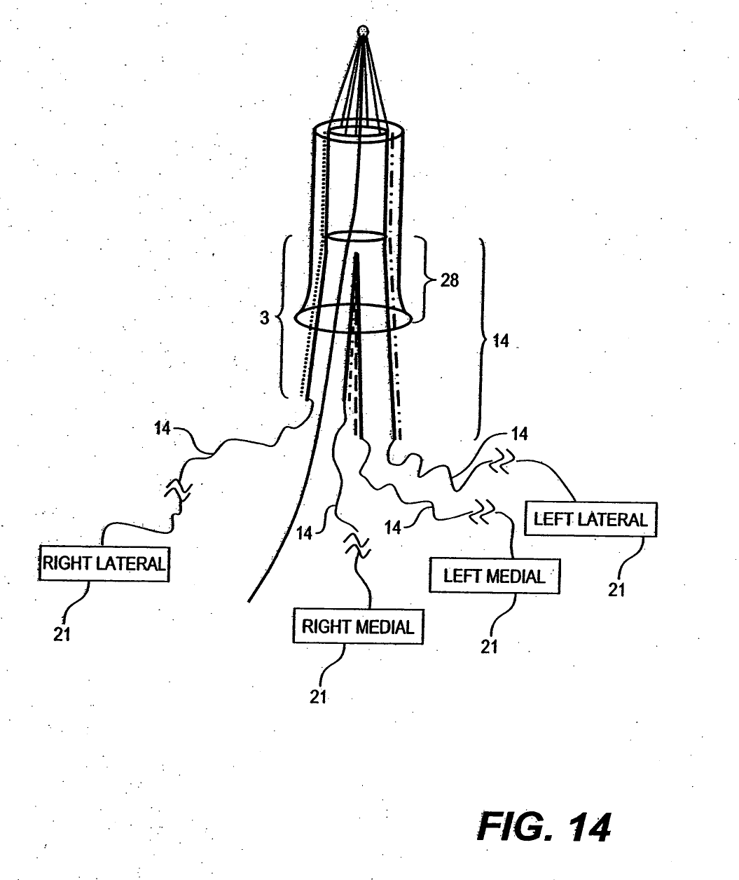

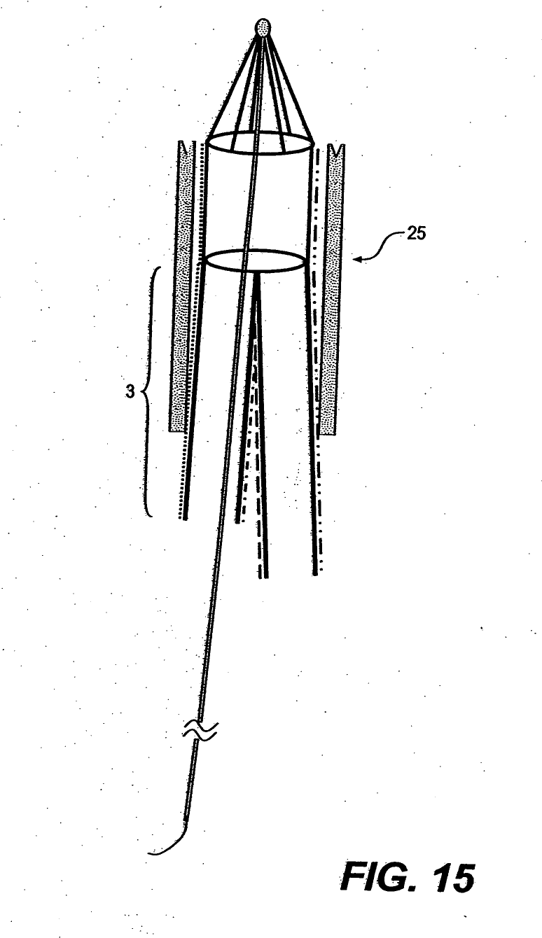

In another embodiment, shown in FIGS. 14 and 15, once the surgical component package is opened by the inserter, the right limb 3 may be cut to the appropriate length, as determined by measurements made from a suitable imaging modality, including, but not limited to, a 3D CT scan. The left limb 4 may be trimmed also. In FIGS. 14 and 16, longer sutures 14 are attached to the graft limbs 3 and 4, and the manufacturer supplied identification tags 21 may be applied or reapplied to the ends of the sutures.

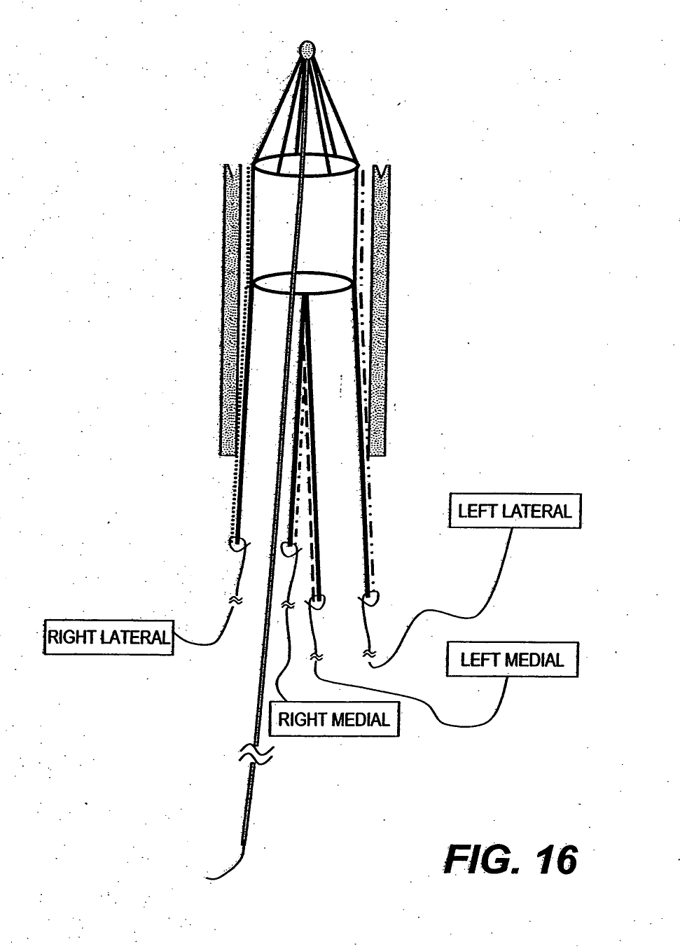

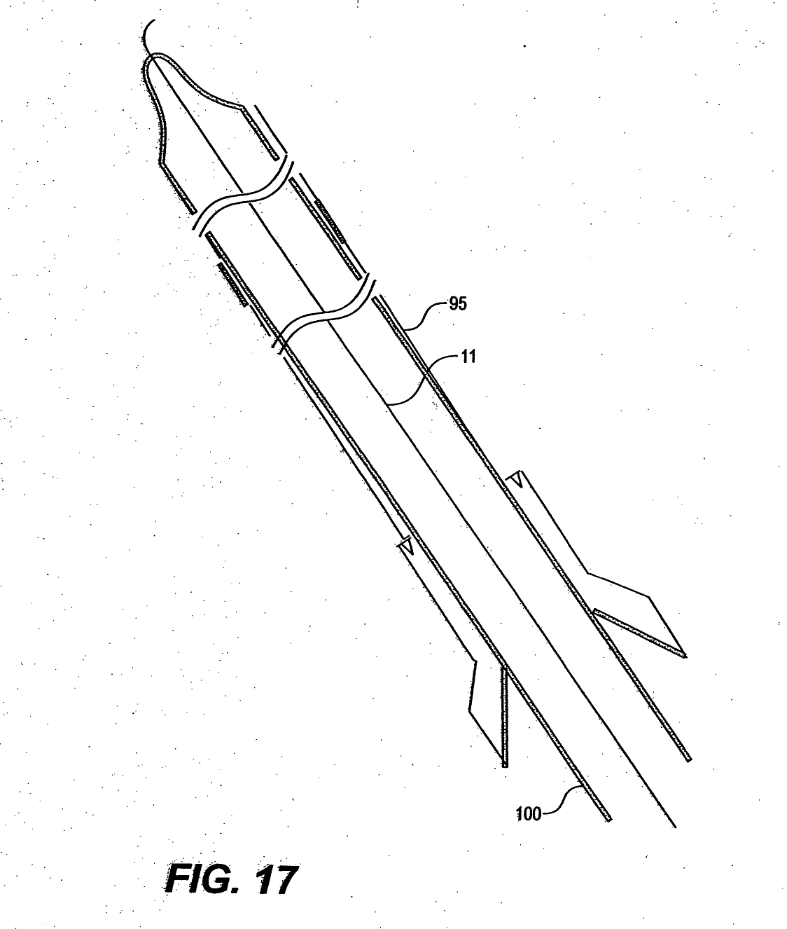

In another embodiment shown in FIG. 17, an introducer sheath 95 with a detachable hemostatic valve 105 and a dilator 100 are illustrated as another embodiment of the present invention. The introducer sheath 95 and dilator 100 may be passed over a support wire 11 from the right or left femoral artery up to the aortic neck. In FIG. 18, the introducer sheath 95 may have a detachable or non-detachable hemostatic valve 105, and the dilator 100 and wire 11 may be removed once the end of the introducer sheath 95 is placed at the level of the aortic neck. The introducer sheath 95 may have a soft or resilient portion 110 that can be clamped off.

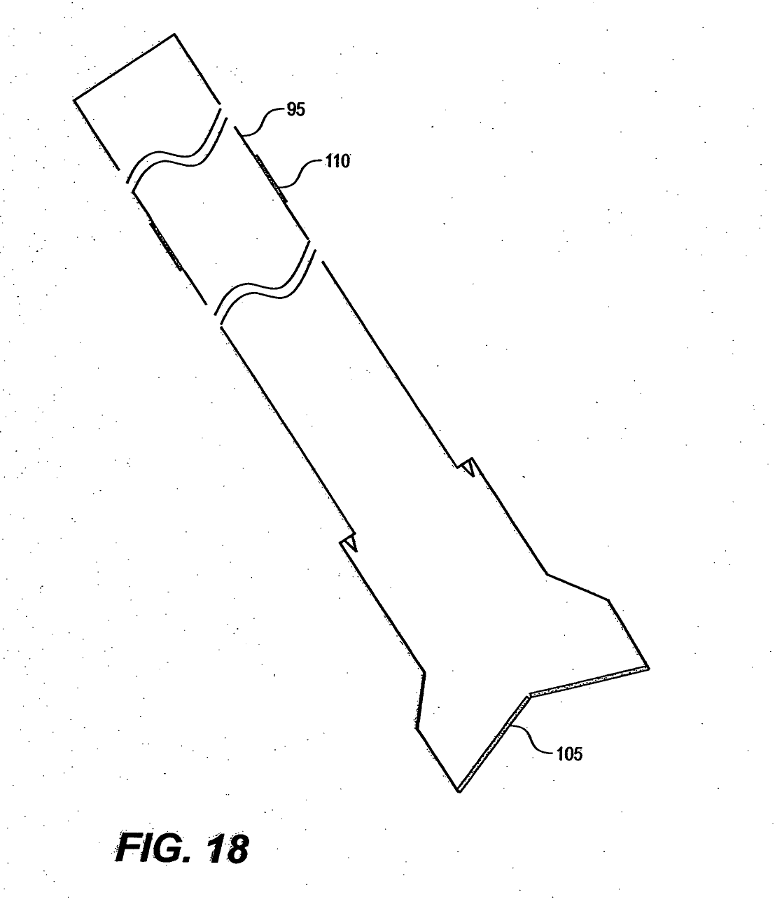

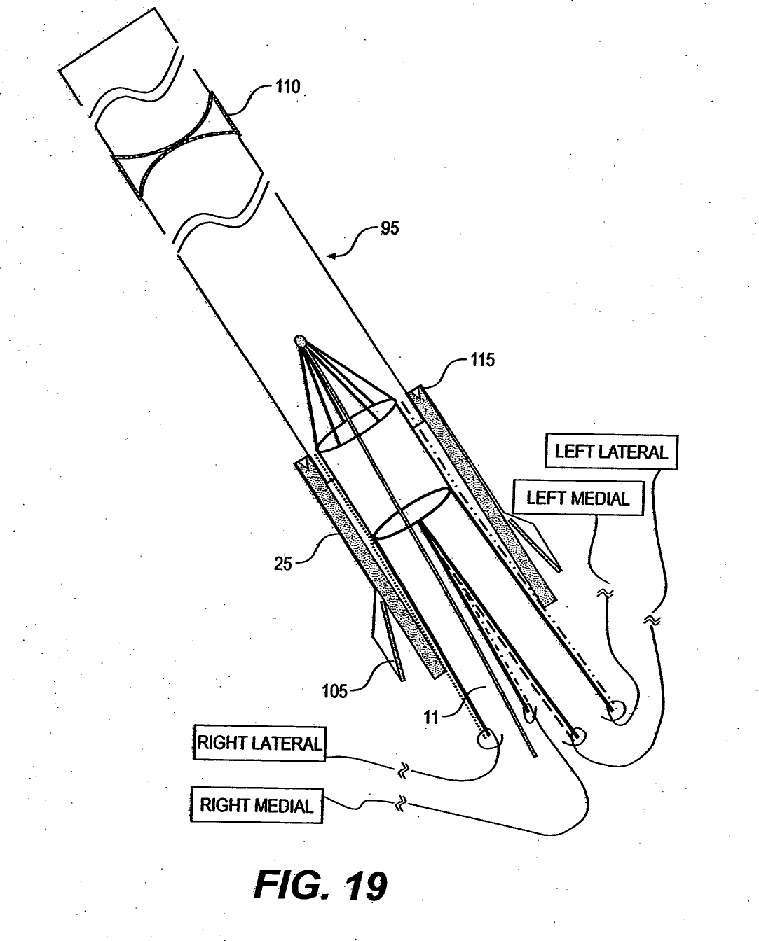

As shown in FIG. 19, the short sheath 25 containing the graft may be introduced into the introducer sheath 95 and advanced until it meets a docking point 115. The lumen of the short sheath 25 and that of the introducer sheath 95 may now be aligned so that easy transfer of the graft from the short sheath 25. The introducer sheath 95 is stationary while the support wire 11 advances the graft within the lumen of the introducer sheath. As the insertion apparatus is being introduced into the introducer sheath 95 the soft or resilient area 110 may be occluded to prevent blood loss while the hemostatic valve 105 is held open.

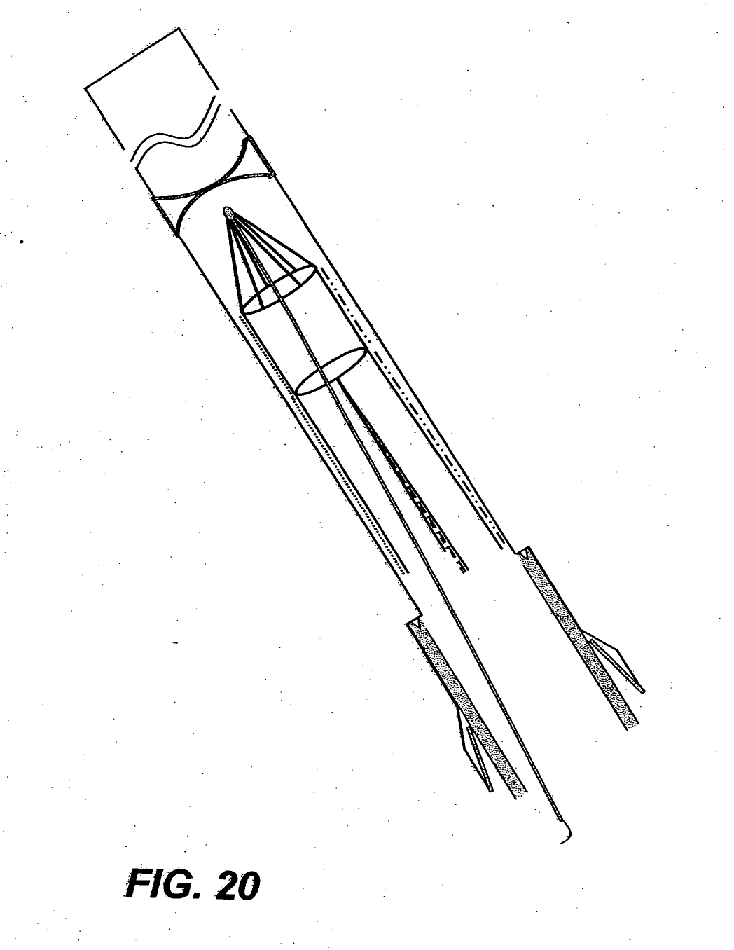

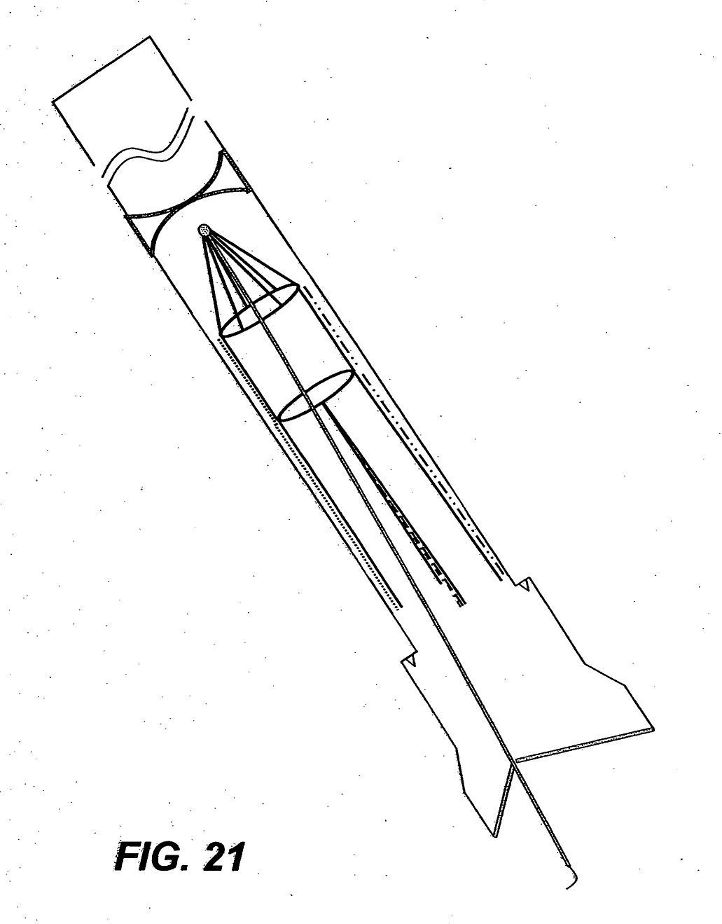

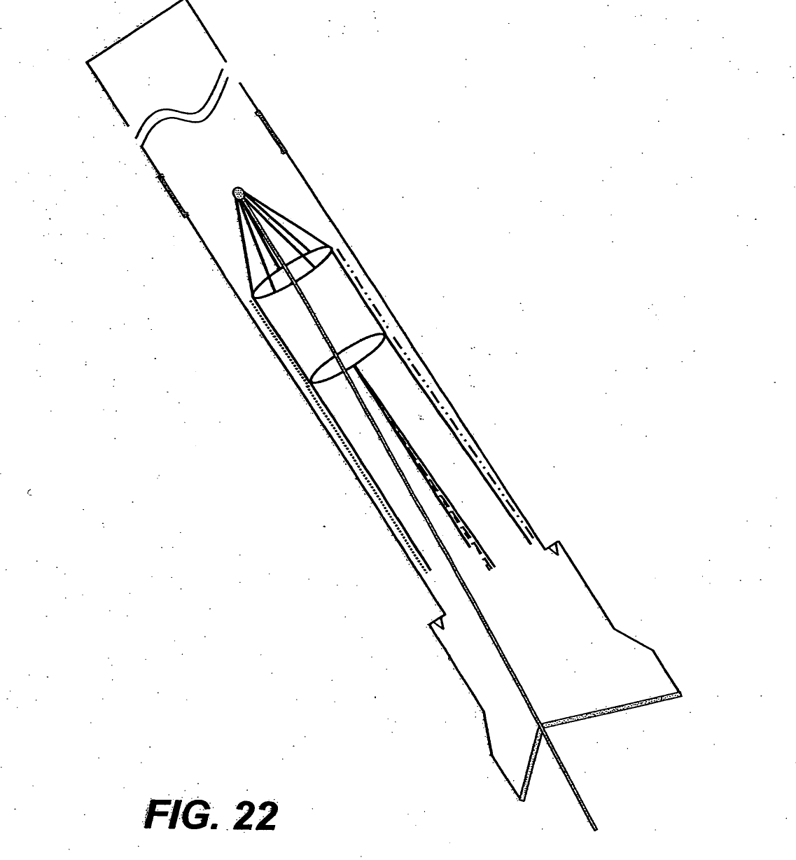

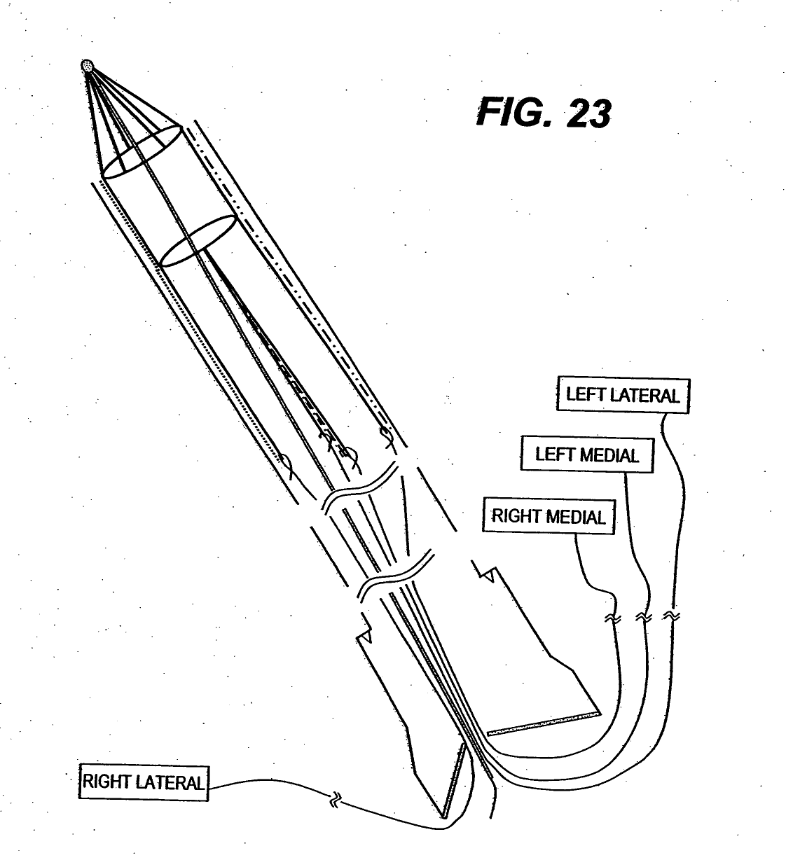

In FIG. 20, by pushing on the support wire 11, the graft may be advanced to the area just before the soft area 110 of the introducer sheath 95. At this point, the graft may be entirely removed from the short sheath 25. In FIG. 21, the short sheath 25 is removed. In FIG. 22, the occlusion mechanism at the soft or resilient area 110 is removed. The graft may be advanced to the end of the introducer sheath 95 and poised for release. FIG. 23 depicts the attachment wires 12 in a spread-out manner when in fact the wires may be tightly compressed against the support wire and may only be released once the restraint of the introducer sheath is removed.

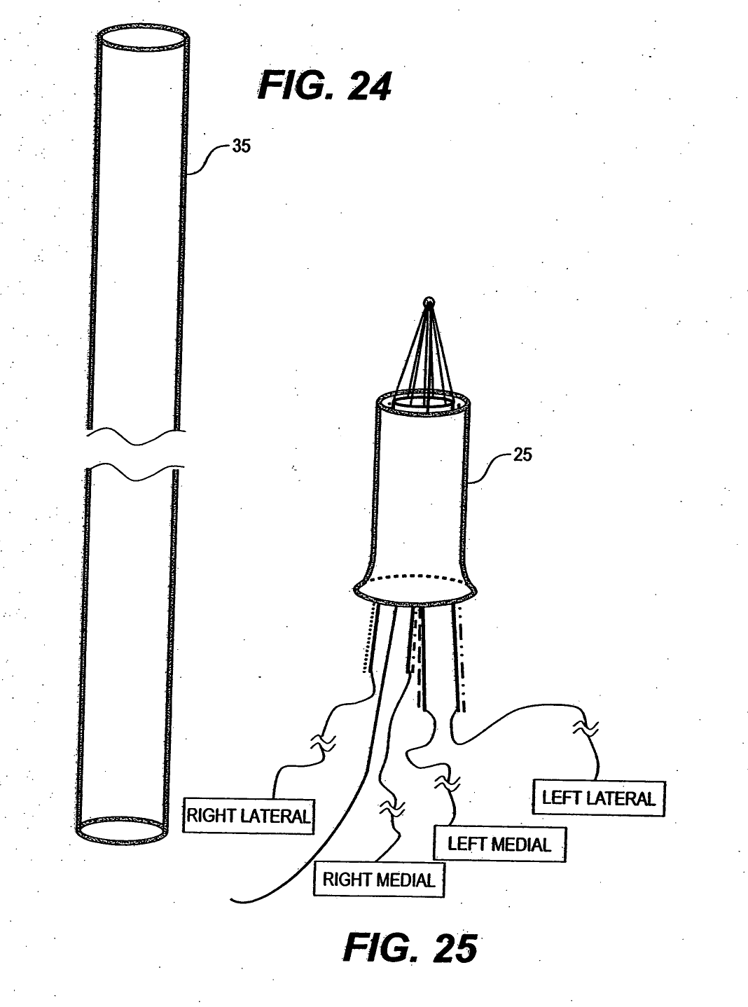

According to another embodiment of the present invention, FIG. 25 depicts a surgical component, which may be supplied to a physician in a short sheath 25 (now shown as opaque). A long delivery catheter 35, as depicted in FIG. 24, may be brought into the sterile field. The long delivery catheter 35 is capable of receiving the contents of the short sheath 25 once the graft limbs are trimmed to the appropriate length and long sutures, with attached tags are reattached to the graft limbs. The long delivery catheter 35 is also capable of insertion into the introducer sheath 95. Thus, the long delivery catheter 35 must be of appropriate length and diameter to facilitate insertion within the introducer sheath to reach the vessel for repair.

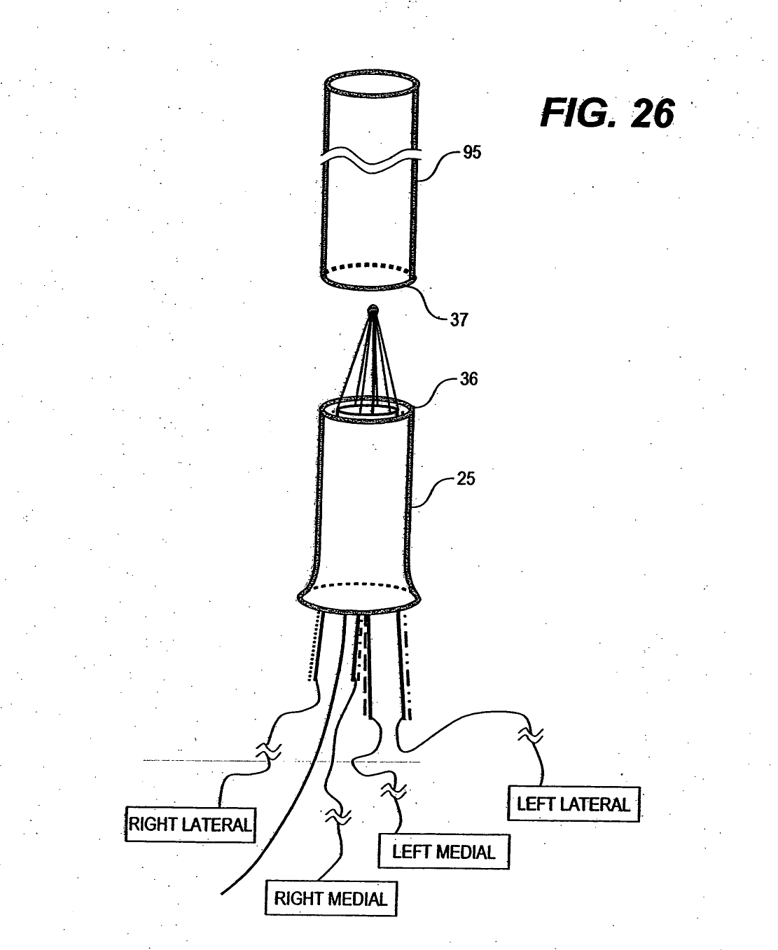

According to FIG. 26, at the end 37 of the long delivery catheter 35 opposite the portion that will be inserted into an artery there may be a connection mechanism so that a port or multiple ports with hemostatic seals can be attached. This will allow the support wire, the sutures attached to the graft limbs, and any other device to be inserted into the patient to either share a hemostatic port or to have separate ports for each or some combination of the above.

As shown in FIG. 26, the top 36 of the short sheath 25 is placed at the end 37 of the long delivery catheter 35 in preparation for the transfer of the graft assembly from the top 36 the short sheath 25 into the long delivery catheter 35.

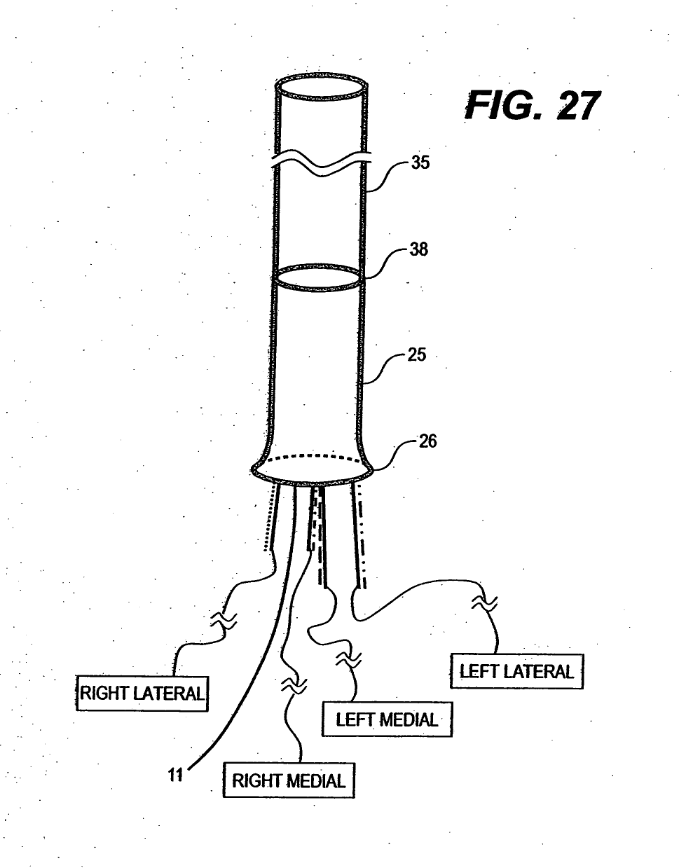

As depicted in FIG. 27, the short sheath 25 may be joined at its top to the long delivery catheter 35 at its bottom to form a junction 38. The junction 38 may be a locking joint that makes the inside appear seamless. The flare on the short sheath 26 assists in helping feed the graft into the short sheath 25 as the graft is pushed into the long delivery catheter 35 by pushing on the support wire 11.

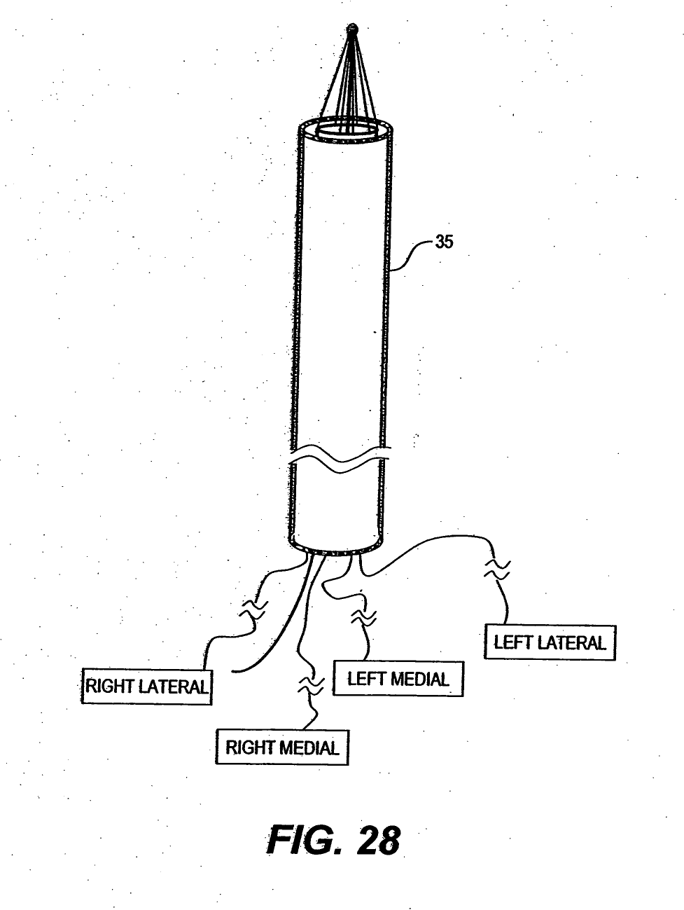

In FIG. 28, the entire graft has been advanced into the long delivery catheter 35 and the short sheath 25 has been removed. This graft transfer may be expedited by having a lubricious inner surface on the sheaths.

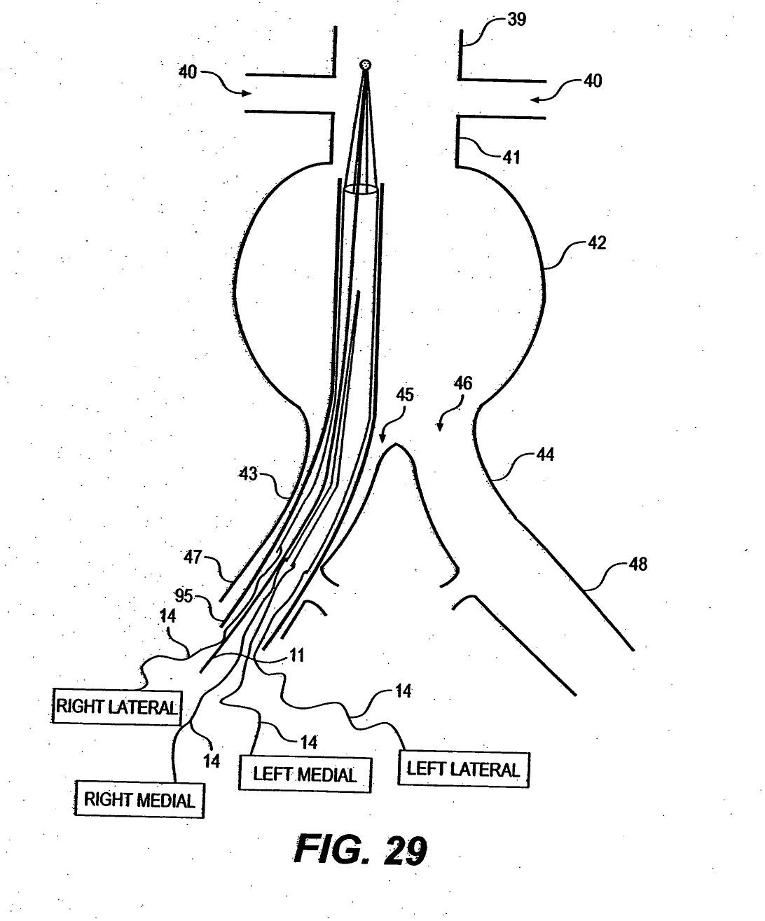

In FIG. 29, the aneurysm diagram depicts a suprarenal aortic portion 39, renal arteries 40, an infrarenal aortic neck 41, an abdominal aortic aneurysm 42, right 43 and left 44 common iliac arteries as well as their orifices 45 and 46, and right 47 and left 48 external iliac and common femoral arteries. According to an embodiment of the present invention, the introducer sheath 95 may already have been inserted into the right femoral artery and advanced up to the level of the aortic neck 41. The support wire 11 and the sutures 14 attached to the graft limbs both exit from the introducer sheath 95. In an alternate embodiment of the present invention, the long delivery sheath 35 is inserted into an introducer sheath (not shown) in the right femoral artery 47 and advanced up to the level of the aortic neck 41. The introducer sheath 95 inserted into the common femoral or external iliac artery 47 over a guide wire at the beginning of the procedure can be left in place for much, though not necessarily all of the procedure.

The end portion of the introducer sheath that is positioned within the aortic neck may be manufactured to be straight when the dilator 100 (FIG. 17) is in place. It may remain straight or it may assume a curved configuration once the dilator 100 is removed. The ability to tip deflect the end of the introducer sheath 95 by controls at the opposite end of the introducer sheath 95 outside the body may be adopted as part of the manufacturing process of the introducer sheath 95. These controls may include manipulating wires through wires passing through longitudinal holes or by means of applying a current or some other force that would change the durometer of the end of the introducer sheath in a manner that would facilitate tip deflection. Such tip deflection would facilitate manipulating the tube portion of the graft 2 into an angulated aortic neck 41.

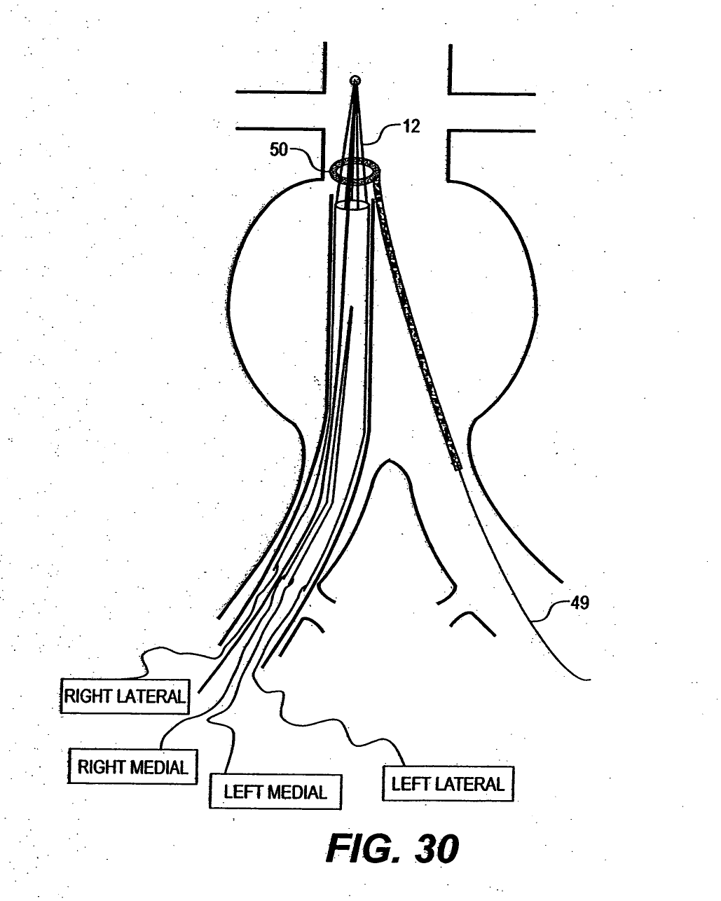

In an embodiment shown in FIG. 30, a snare catheter 49 may be inserted through the opposite femoral artery, in this example, the left femoral artery 48 sheath (not shown) and the snare component 50 may be advanced over the attachment wires 12. Use of a snare may reduce the introducer sheath's outside diameter. If space is available within the right larger introducer sheath, there may be a multiplicity of ways, for example, as referenced in the description of FIG. 7, to build in an expansion/retraction system to handle the expansion and retraction of the attachment wires 12.

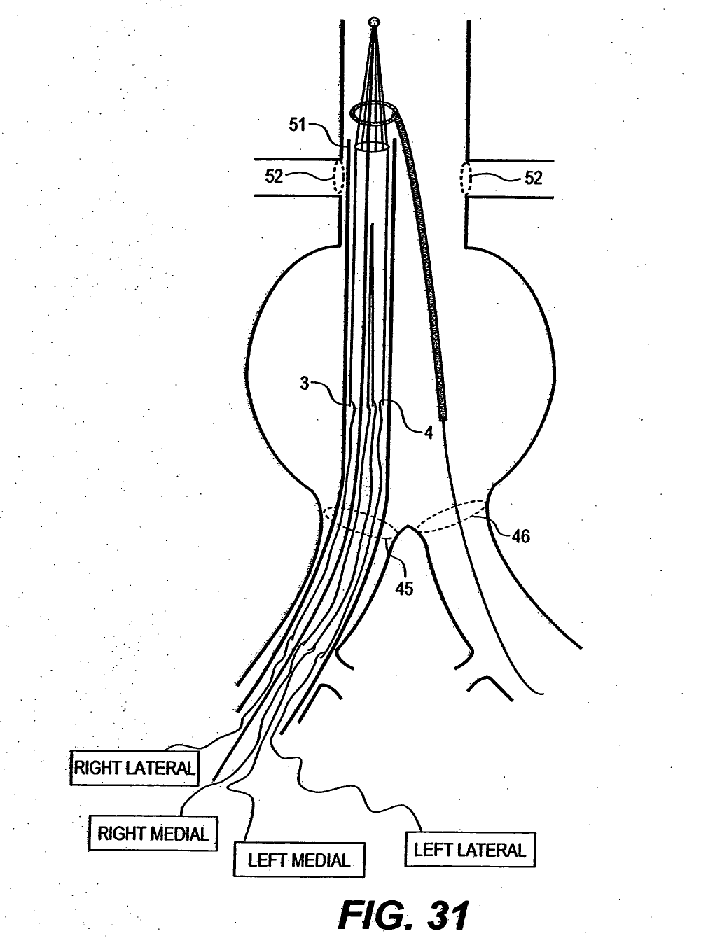

As shown in FIG. 31, the top of the introducer sheath 95 may be brought above the orifices 40 of the renal arteries so that the ends of the graft limbs 3 and 4 are above the orifices 45 and 46 of the common iliac arteries.

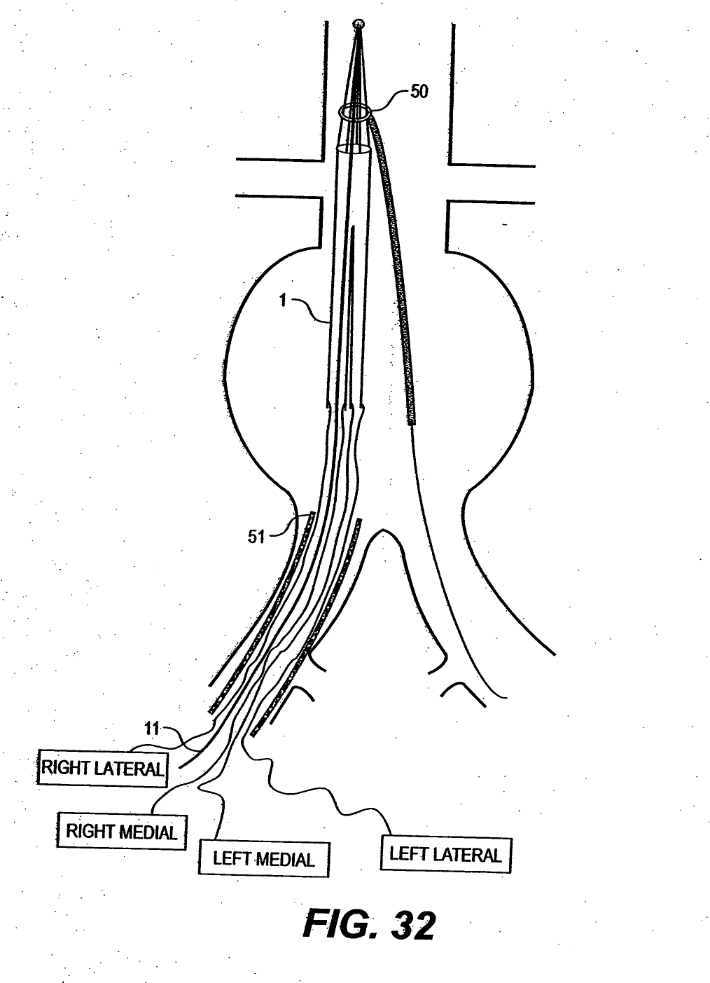

In FIG. 32, the top 51 of the introducer sheath 95 may be pulled back while the support wire 11 is held steady so that the entire graft 1 is released from the introducer sheath. In this embodiment the snare component 50 prevents the top of the graft from expanding. As referenced in the description of FIG. 7, a catheter could also be passed over the support wire 11 and connected to a mechanism at the bulb 27 that would allow control of the angulation of the attachment wires 12 which would make the use of the snare optional.

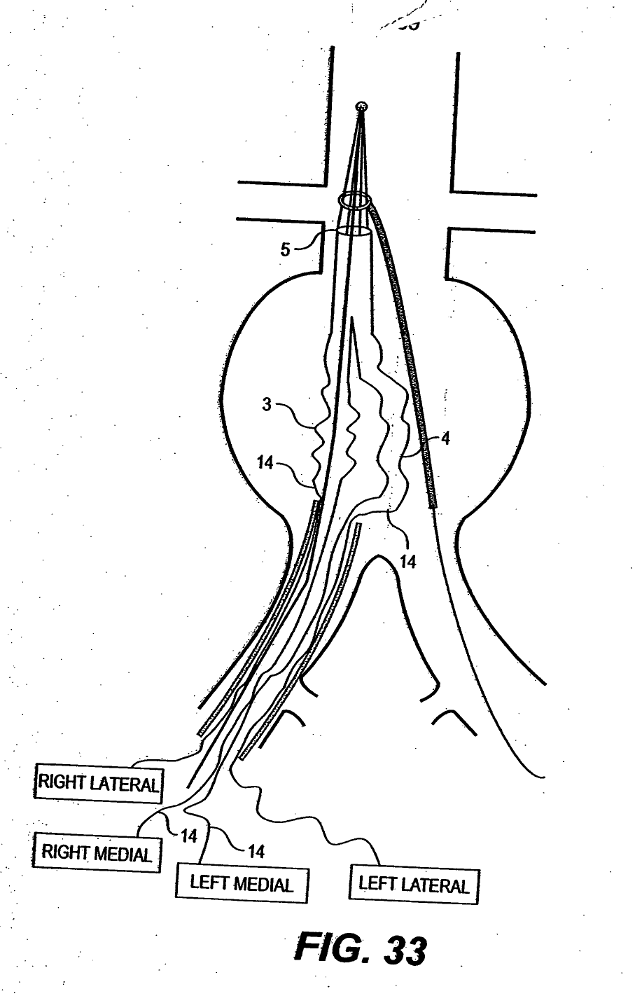

According to FIG. 33, the top of the graft 5 may be placed within the aortic neck. As the graft is released, the right 3 and left 4 limbs of the bifurcation graft may not be under tension from the attached sutures 14 and may be contained within the aneurysm.

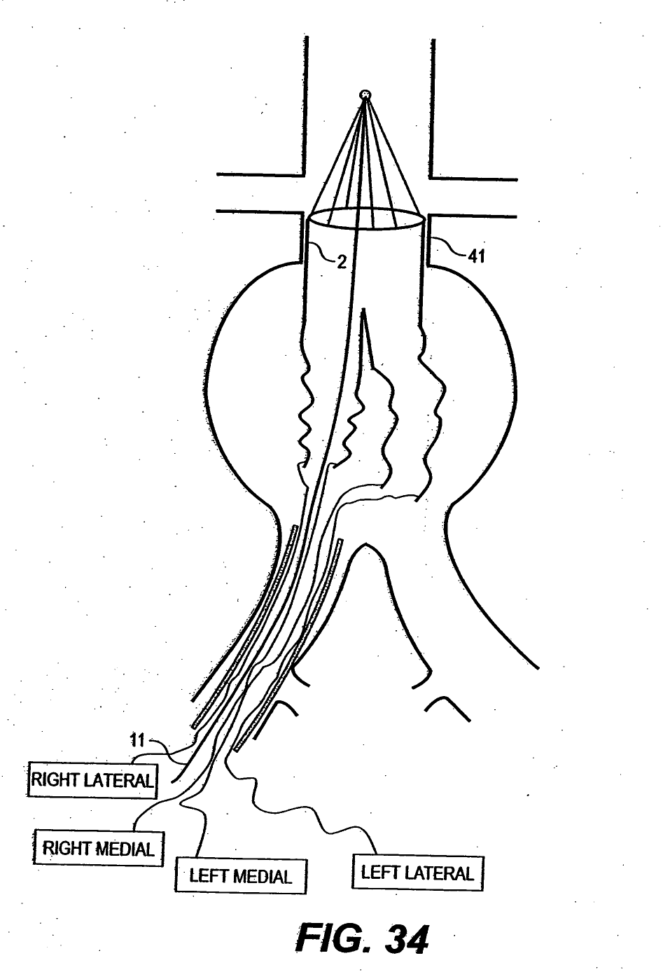

In FIG. 34, the snare component 50 is removed and, by holding the support wire 11 steady, the tube portion 2 of the graft remains in position within the aortic neck 41. The attachment wires 12, for the first time unrestrained, may hold the graft open. In an embodiment of the invention, the aorta is not occluded with the release of the graft. This approach may be less dangerous than techniques that require intermittent aortic occlusion because the heart is not subjected to large fluctuations in systemic vascular resistance. If the aorta is occluded near the level of the renal arteries, the pressure that the heart has to pump against is high. Indeed, systemic vascular resistance may be altered minimally with the approach presented in embodiments of the present invention.

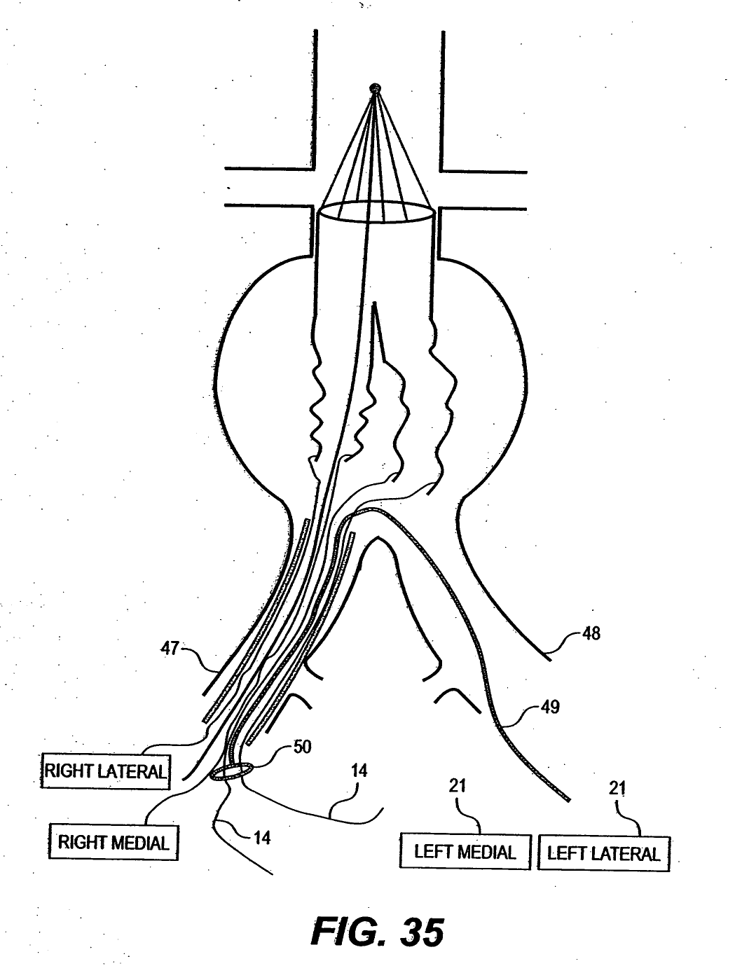

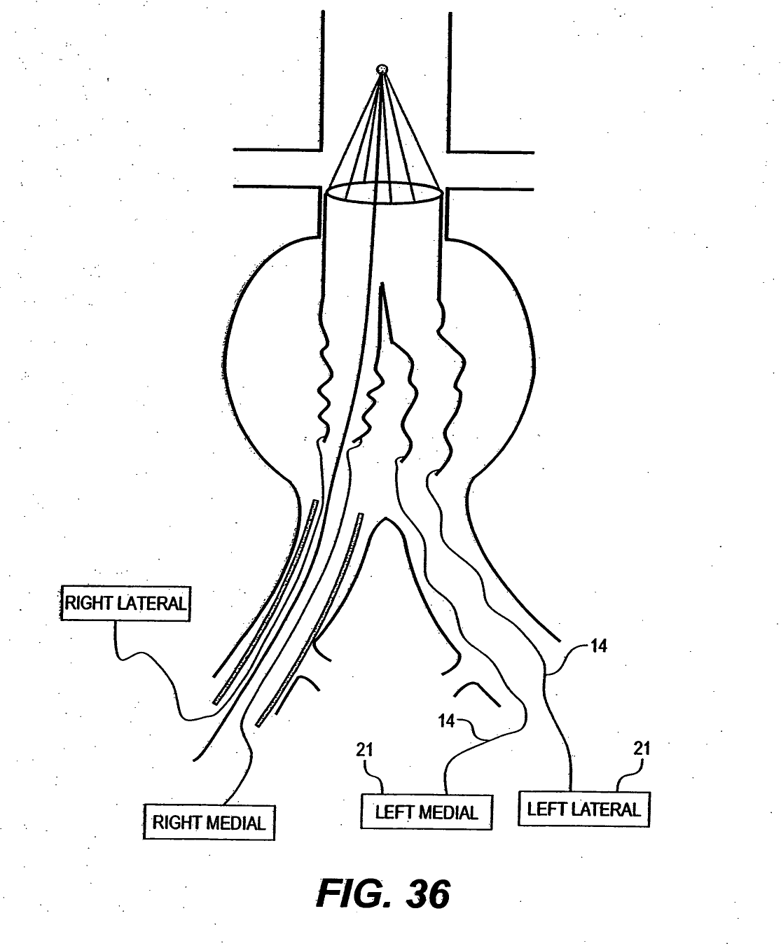

As shown in FIG. 35, in this example, a snare catheter 49 is passed from the left femoral artery 48 out the right femoral artery 47. The sutures 14 attached to the left limb 4 of the graft 1 may be placed within the snare component 50. In FIG. 36, the identification tags 21 for the left medial and left lateral have been detached and are reattached once the sutures have been passed to the left side.

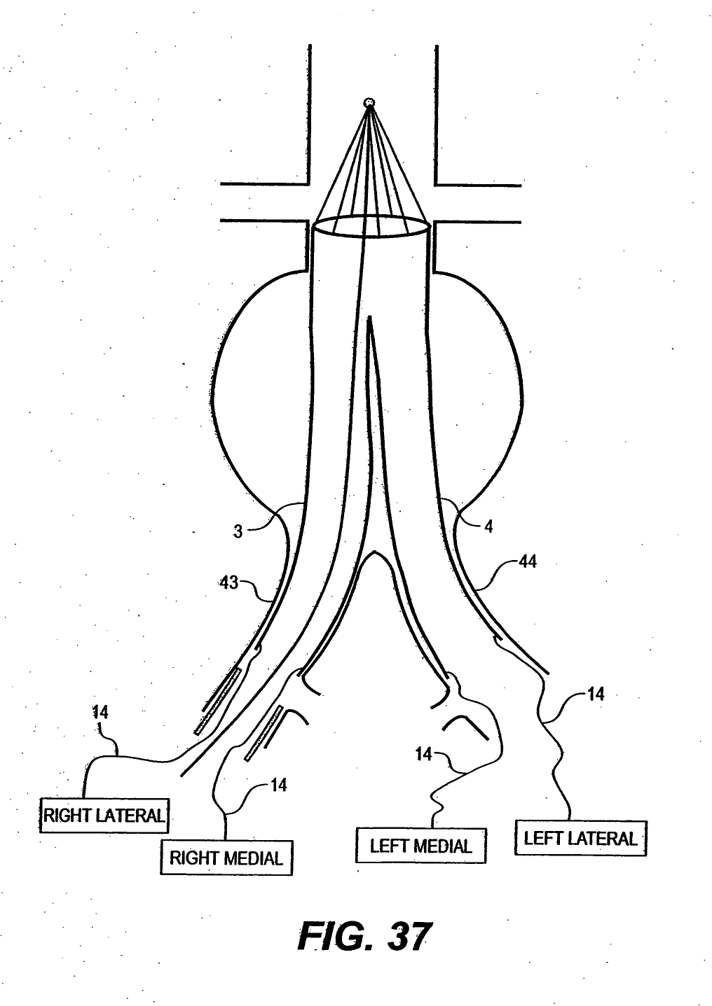

In FIG. 37, tension may be placed on the sutures 14 attached to the left limb 4 and also to the sutures 14 attached to the right limb 3 so that the limbs are brought down into the left 44 and right 43 common iliac arteries.

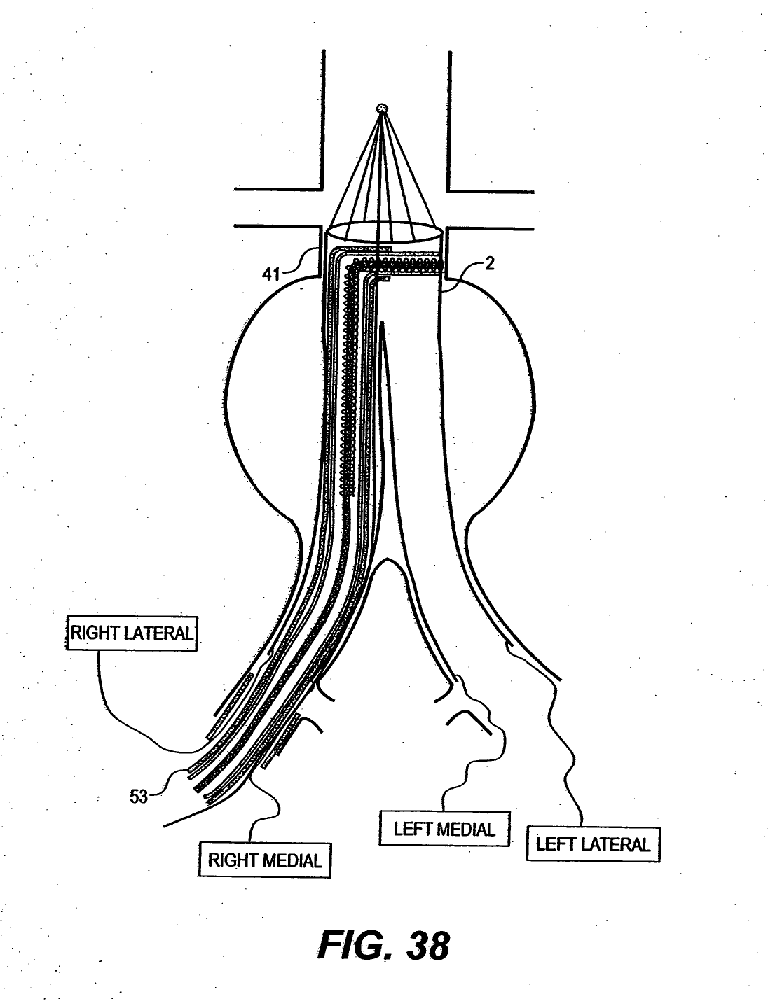

In an embodiment shown in FIG. 38, a fastener delivery catheter 53 may be inserted and positioned within the aortic neck 41 adjacent to or near the top of the tube portion 2 of the graft in order to initiate insertion of the fasteners 54. The fastener delivery catheter 53 disclosed in U.S. application Ser. No. 09/783,313 filed Feb. 15, 2001, the disclosure and the subject matter is hereby incorporated herein by reference. The fastener catheter 53 may also provide any suitable type of fasteners, including, but not limited to, metal alloy, plastic, suture, wire, or any other suitable fastening mechanism.

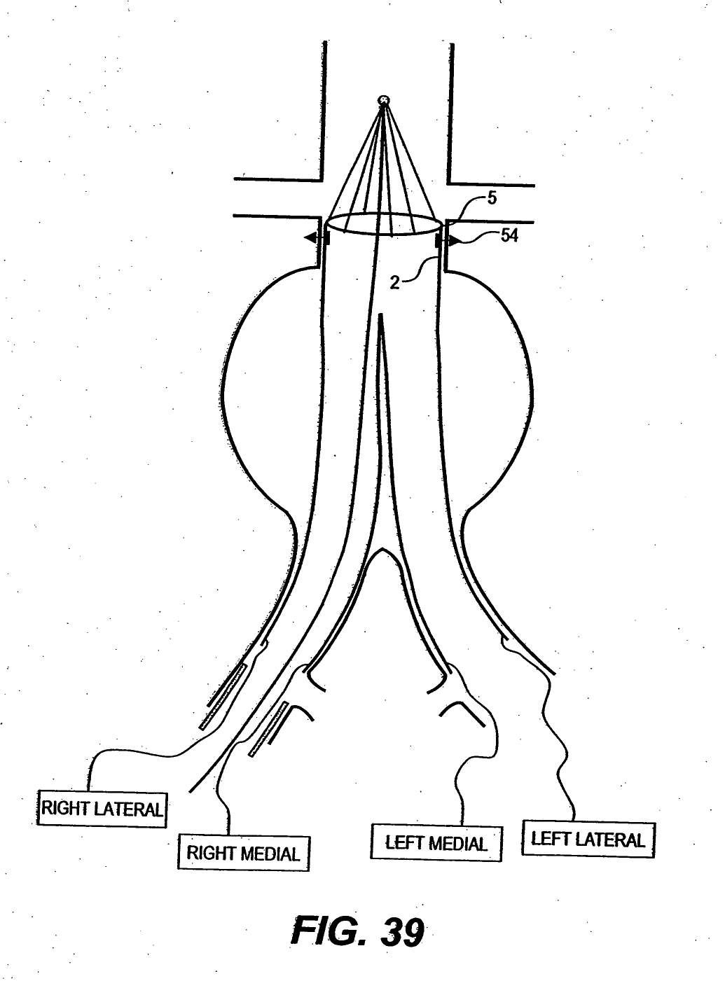

In an embodiment shown in FIG. 39, fasteners 54 may be inserted circumferentially around the top 5 of the tube portion 2 of the graft 1.

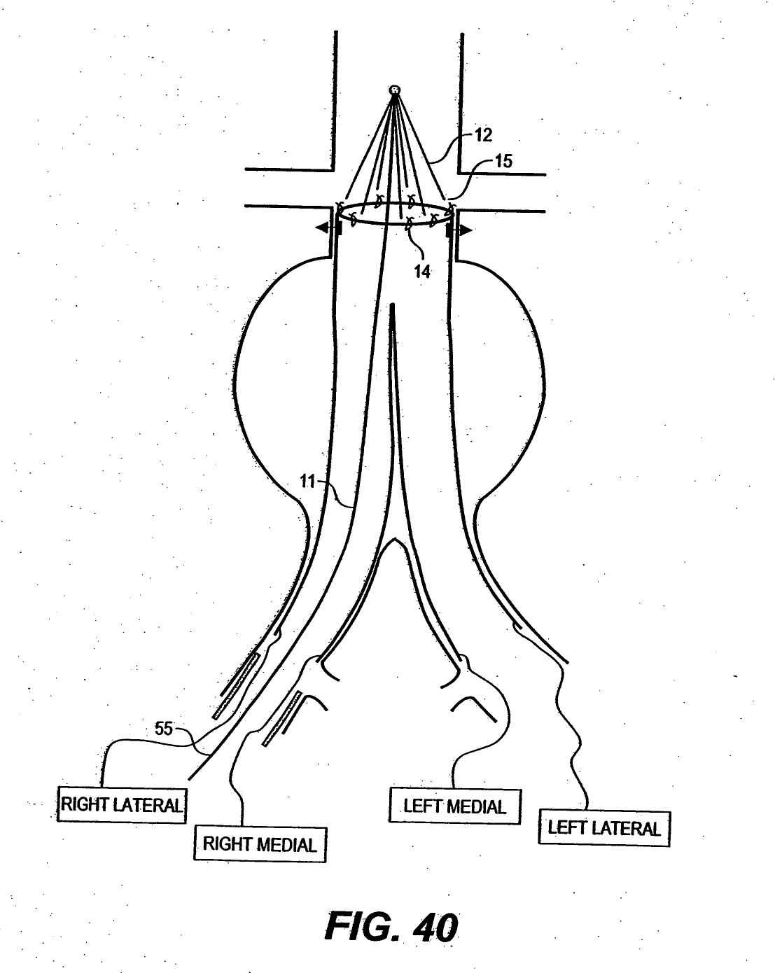

In an embodiment shown in FIG. 40, an electrical current may be applied to the support wire 11 by an actuator 55 outside the body of the patient. This causes the filaments attached to the attachment wires 12 to heat and sever the attached sutures 14. FIGS. 4, 5, and 6 detail an embodiment of the release mechanism using a current to increase temperature. Any suitable means may be used to effect release

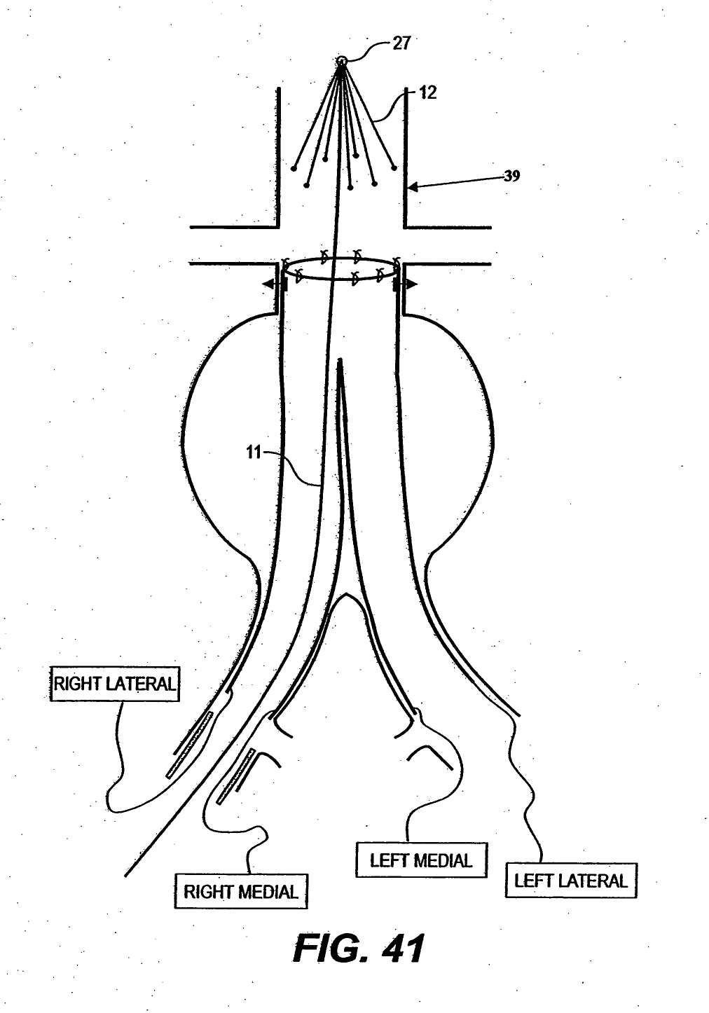

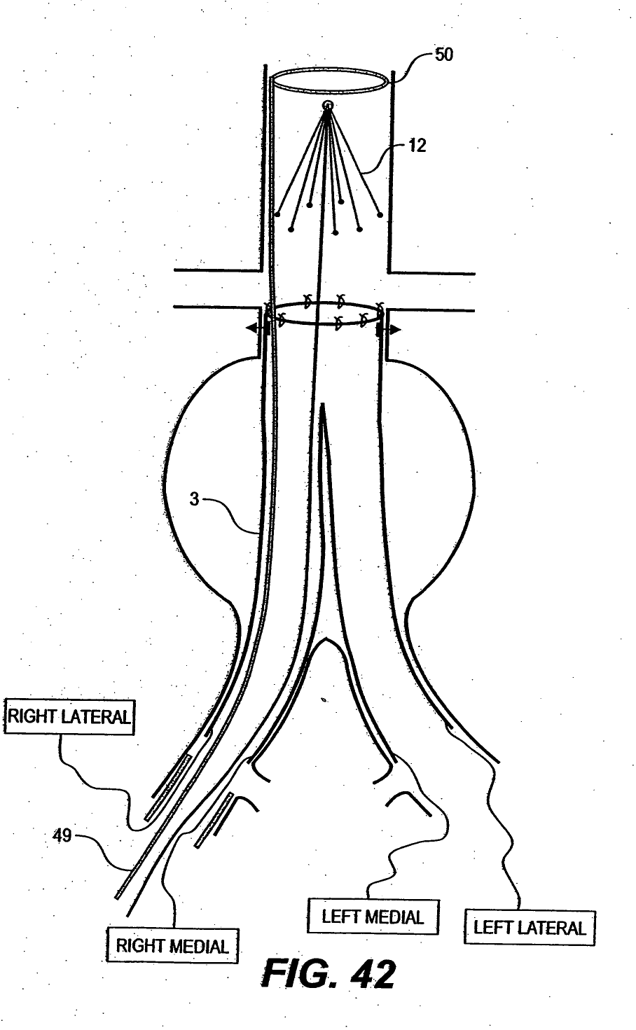

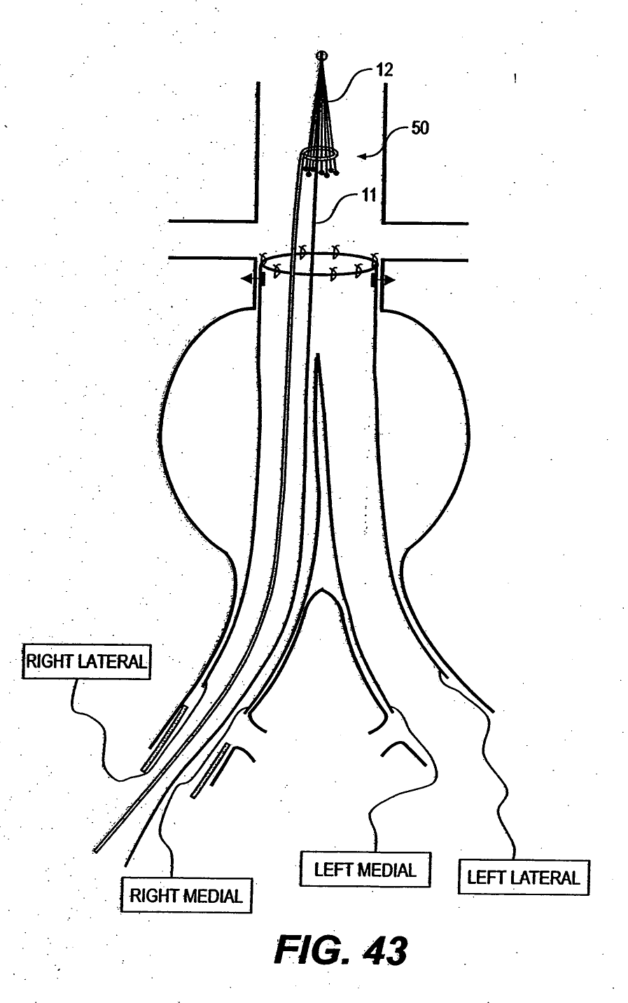

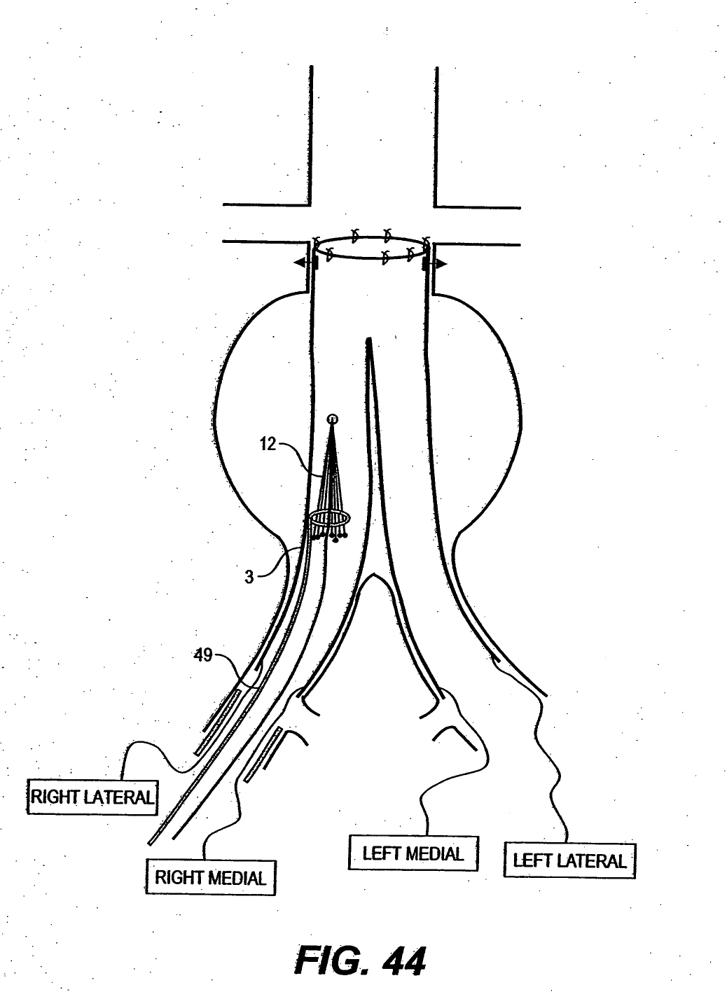

FIGS. 41 through 44 depict an embodiment of the release mechanism, and a method to remove the attachment wires 12 and the support wire 11 through the use of the snare component 50. In FIG. 41, by advancing the wire 11, the bulb 27, and the attached attachment wires 12 may be advanced into the suprarenal aorta 39. In FIG. 42, the snare catheter 49 is advanced through, by way of example, the right limb of the graft 3 and the snare component 50 is placed above the attachment wires 12. As shown in FIG. 43, the snare component 50 may be tightened around the attachment wires 12 so that they may be brought close to the support wire 11. In FIG. 44, the snare catheter 49 may be withdrawn through the right graft limb 3 bringing the attachment wires 12. As referenced in the description of FIG. 7, there can be multiple other methods of facilitating the transition between an expanded and a contracted configuration of the attachment wires.

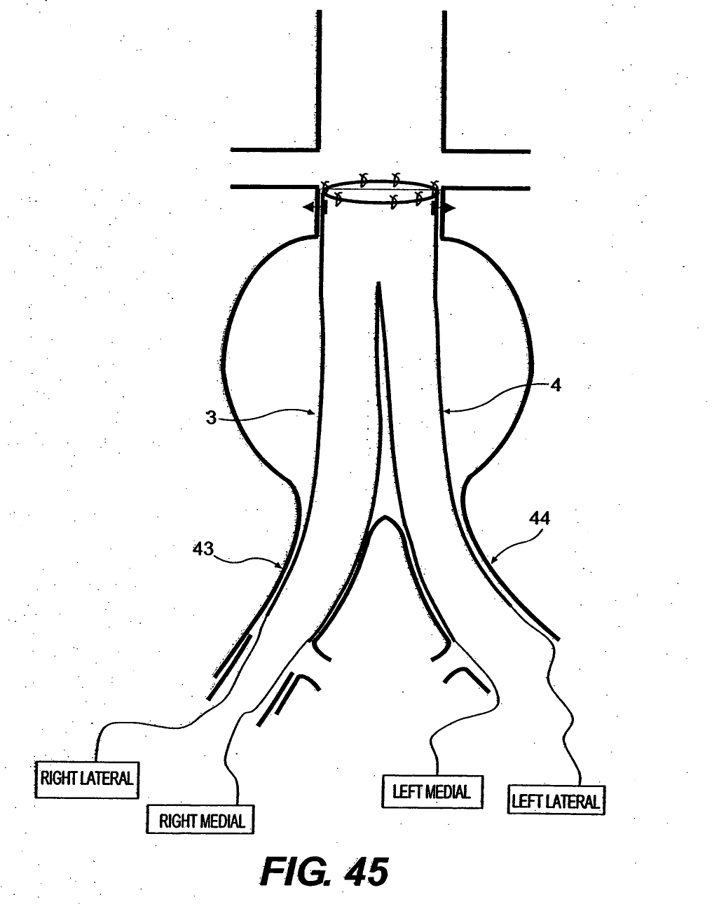

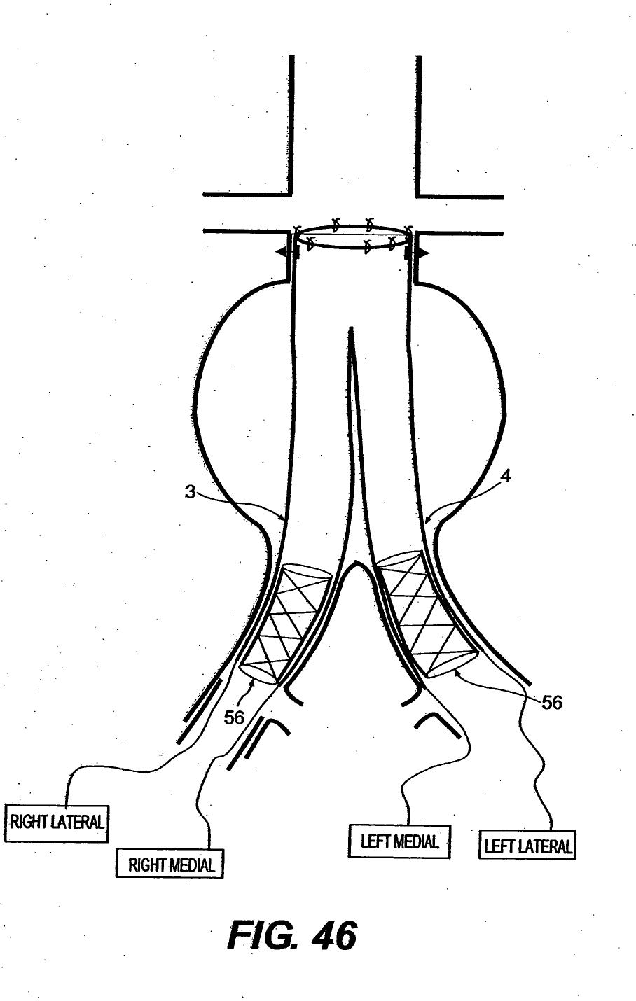

FIGS. 45-47 illustrate securing the graft limbs to the left 44 and right 43 common iliac arteries. As shown in FIG. 45, the attachment wires have been removed and the right 3 and left 4 graft limbs are now ready for attachment within the right 43 and left 44 common iliac arteries. In FIG. 46, two stents 56 have been inserted into the distal graft limbs 3 and 4 to provide an attachment means to the distal graft limbs.

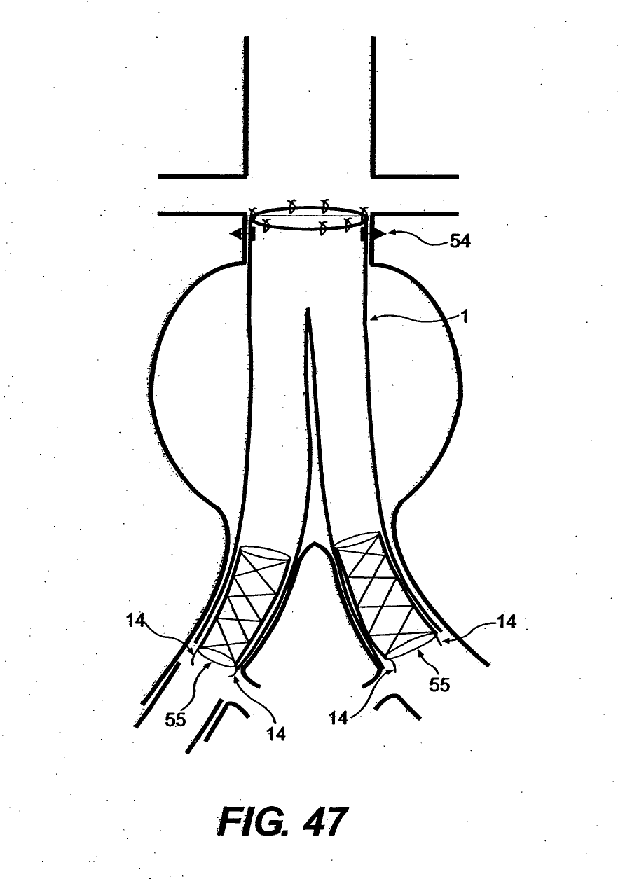

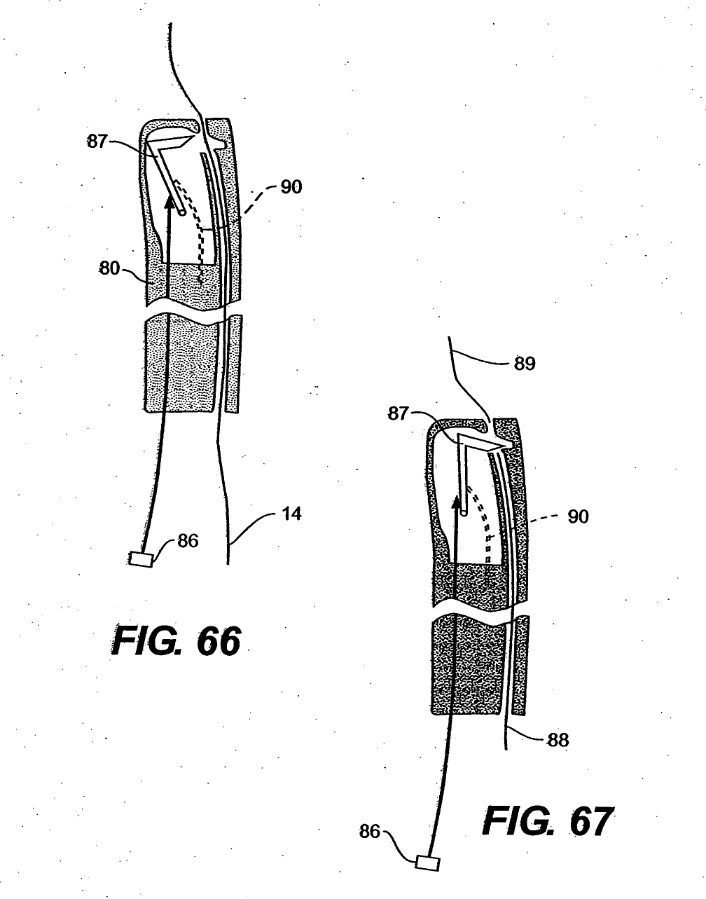

In FIG. 47, the sutures 14 have been cut with a suture cutter 80. The suture cutter 80 is shown in FIGS. 66 and 67, and described in more detail below. The graft 1 is now securely attached to the aortic neck with fasteners 54 and to the common iliac arteries with stents 55.

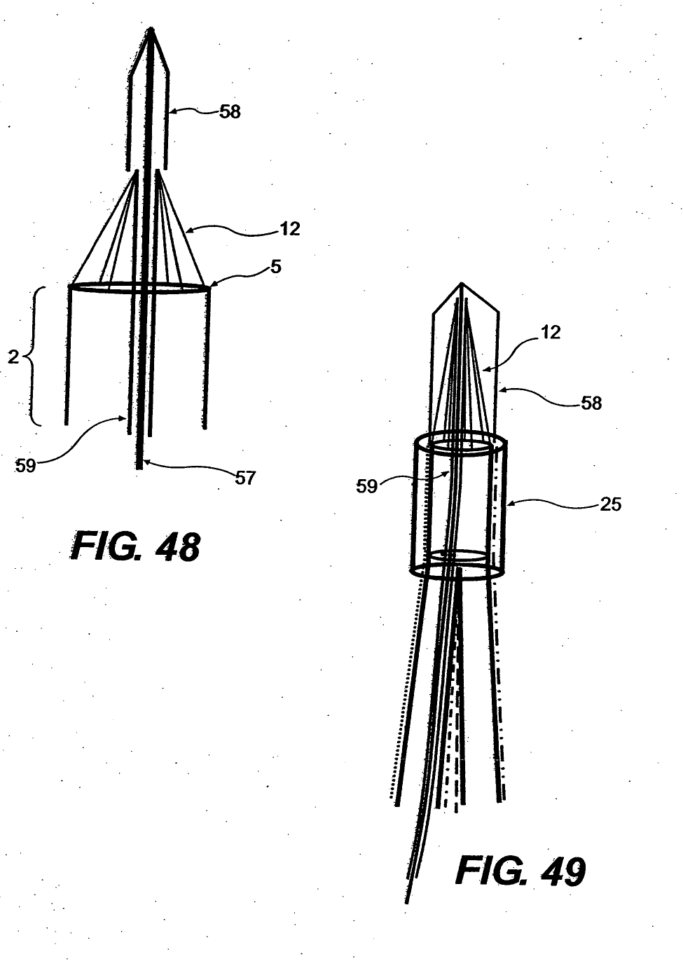

Instead of using the snare technique as shown in FIGS. 30-33 and FIGS. 42-44, an alternative embodiment uses a sheath as depicted in FIGS. 48, 49, and 54. The attachment wires 12 are attached to the top 5 of the tube portion 2 of the graft 1. The graft may be a supported graft or an unsupported graft. The insertion mechanism is composed of a central wire 57. The central wire 57 may be bendable by the inserter to facilitate inserting the graft into an angled aortic neck. The central wire 57 attaches to a containment sheath 58 above the top 5 of the tube portion 2. Instead of the attachment wires 12 being attached to the support wire 11, they now are attached to a catheter 59 surrounding the central wire 57. The central wire 57 and the support wire 11 function in a similar manner and may be made of the same material. FIG. 48 depicts the attachment wires 12 unsheathed.

The embodiment shown in FIG. 49 illustrates a way of supplying the graft positioned within a very short sheath 25 similar to the method described in FIG. 12. In this instance, however, the attachment wires 12 are enclosed and compressed toward the catheter 59 by the containment sheath 58. Note that the manufacturer can supply the graft with just the tube portion in a short sheath 25. The short sheath 25 may extend from the tube portion 2 to a portion over the limbs 28, as depicted in FIG. 12 or may extend just over the tube 2, as depicted in FIG. 49.

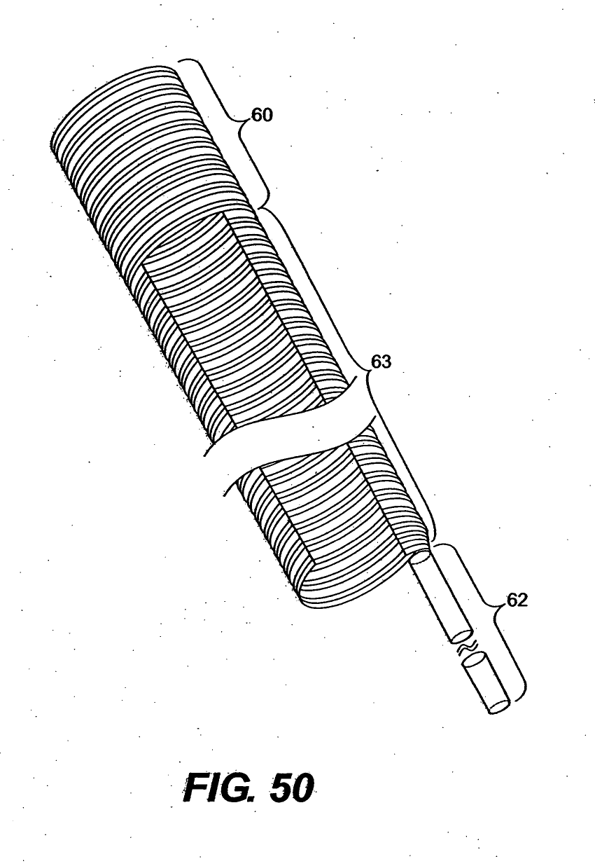

FIGS. 50, 51, and 52 provide additional embodiments of the present invention.

FIG. 50 provides a depiction of a very short sheath 25 connected to an open or partial sheath 63. If desired, this can be connected to a solid component such as, but not limited to, wire or a synthetic, such as plastic or an open catheter 62.

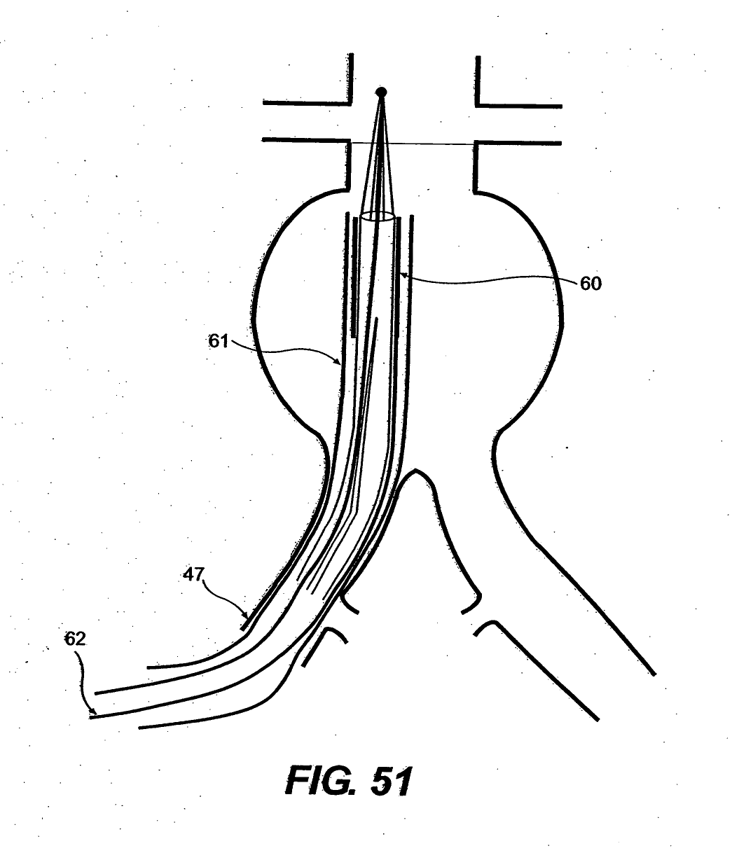

FIG. 51 depicts such a sheath in position within an aortic aneurysm. This example illustrates the tube portion 2 of the graft and attached to some wire 62 or partial catheter within it. Note that the manufacturer can supply the graft with just the tube portion in a very short sheath. This very short sheath 60 could allow the inserter to shorten the graft limbs to the desired length as shown in FIG. 14 and then allow the graft 1 to be inserted directly into an introducer sheath 61.

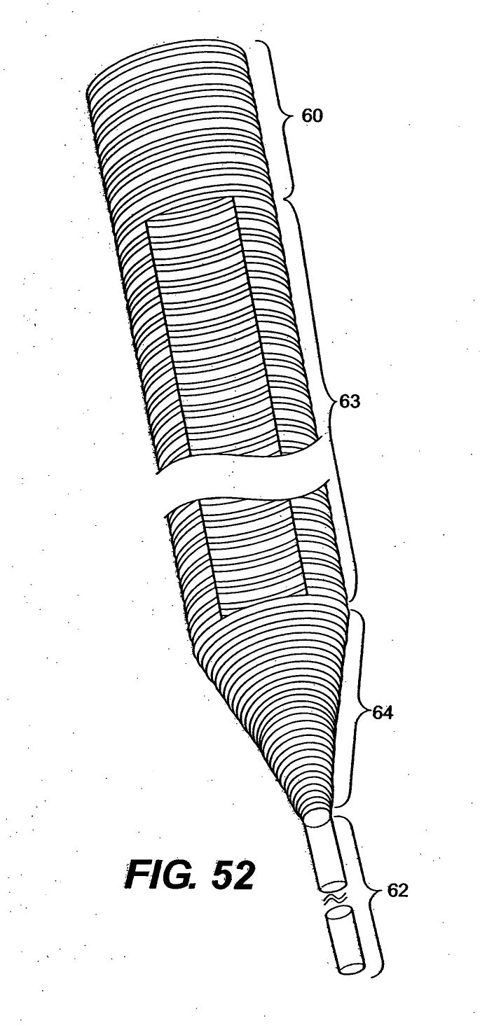

FIG. 52 is another embodiment of a sheath design analogous to that depicted in FIG. 50 though the catheter 62 would most likely have a lumen to allow a mechanism to pass through in order to deploy the graft 1. This is a depiction of closed circumferential sheath 60 that becomes a partial or open sheath 63 that cones down 64 to an open catheter 62.

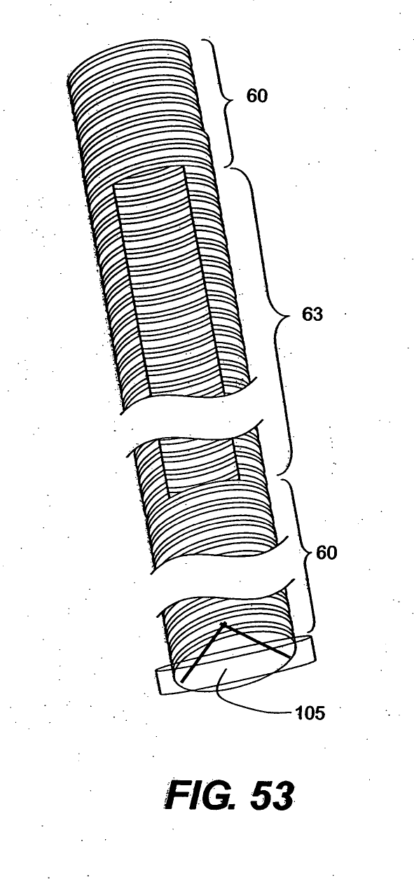

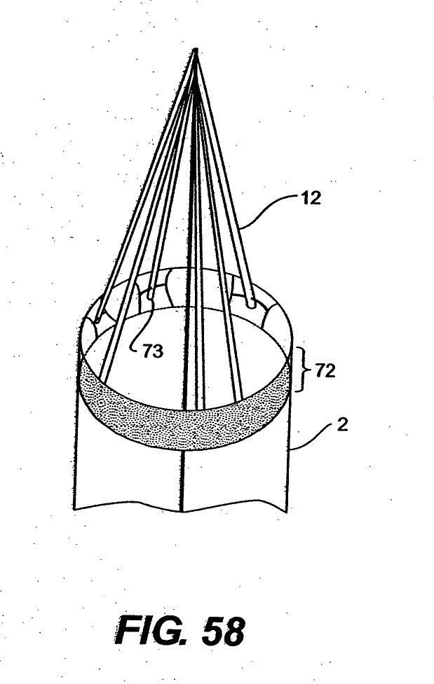

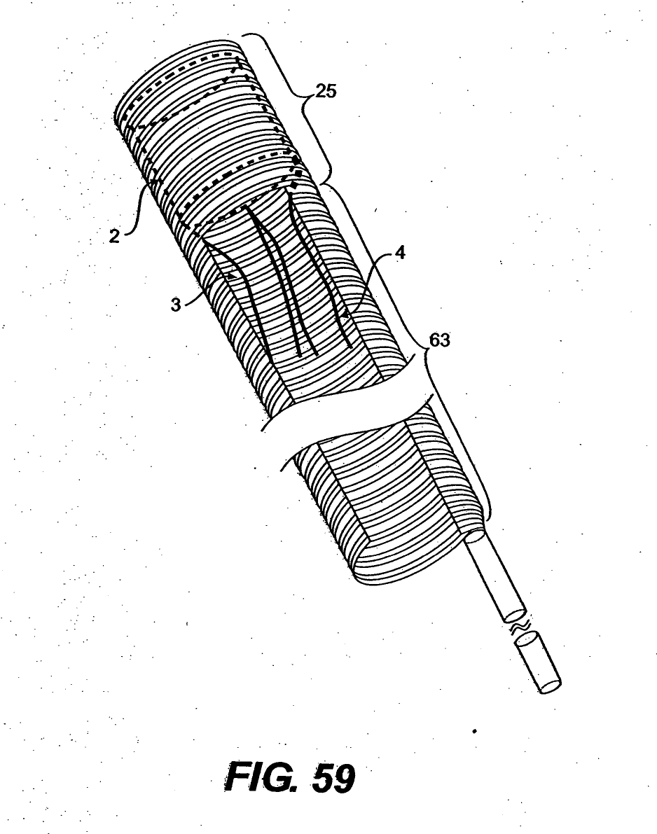

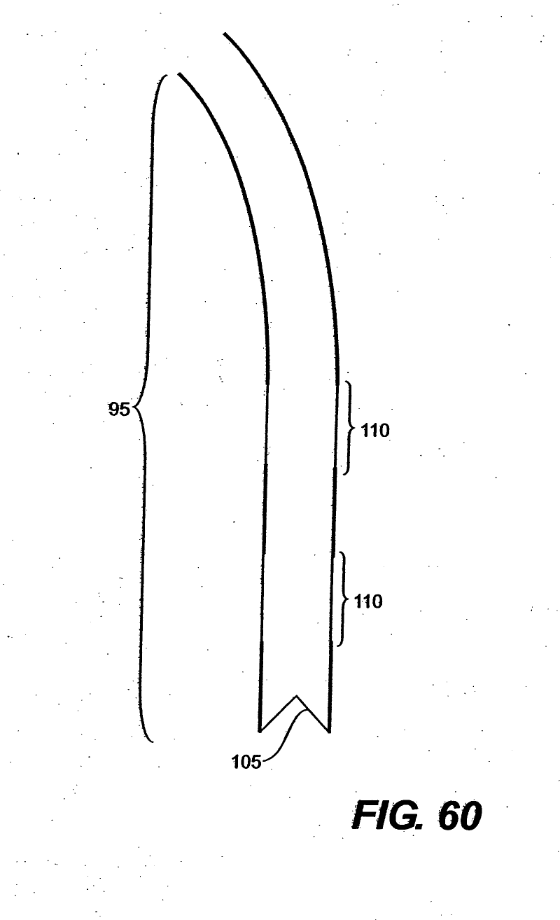

FIG. 53 depicts a closed circumferential sheath 60 that becomes a partial or open sheath 63 that then resumes its closed circumferential sheath configuration 60 that then may end in a hemostatic valve 105. Although not shown, the introducer sheath could be supplied with one or more soft or resilient areas, as depicted in FIG. 59, and the hemostatic valve could be replaced with a connector, as depicted in FIG. 60.

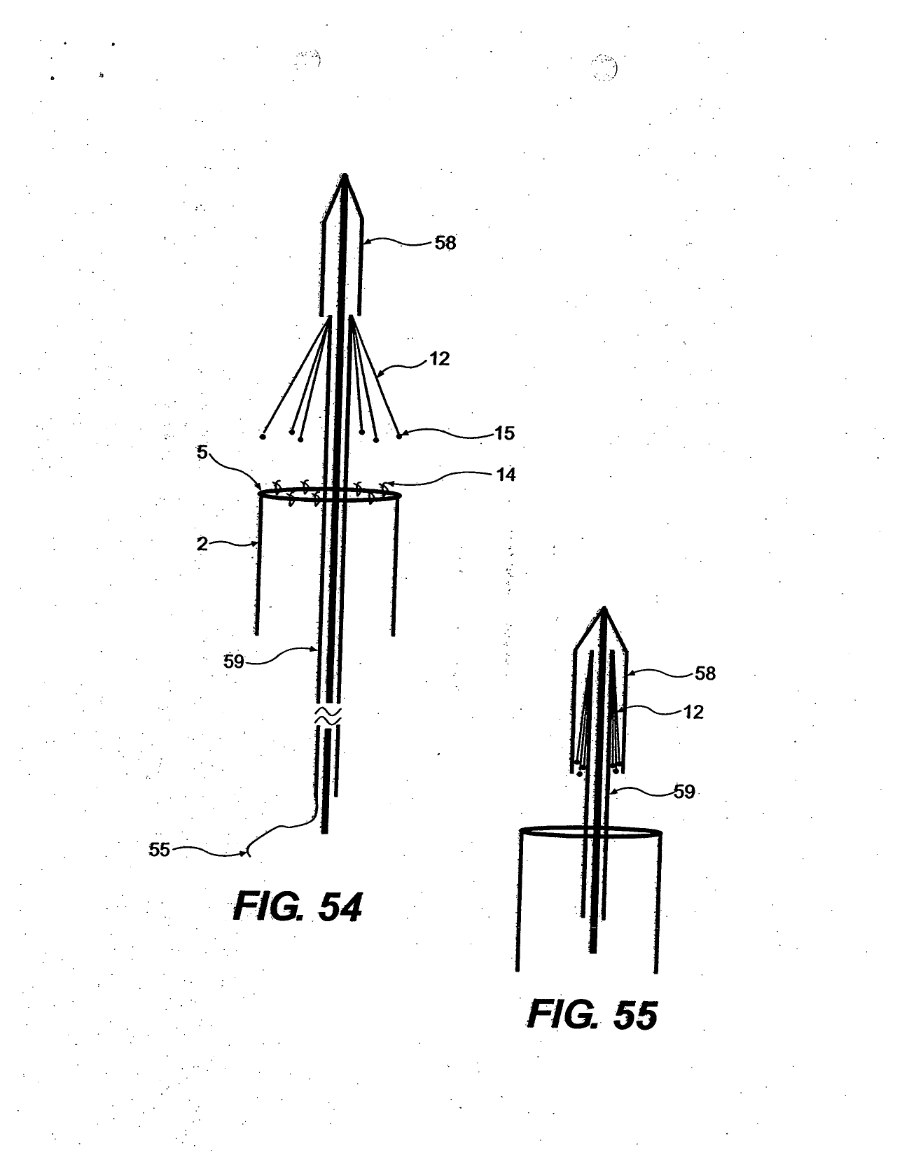

In FIG. 54, an electrical current is applied by an actuator 66 within the catheter 59 when it is desired to release the attachment wires 12 at the small attachment mechanism ball 15 attached to the sutures 14 that are in turn attached to the top 5 of the tube portion 2 of the graft. This release mechanism is also shown in FIGS. 4, 5 and 6. This current triggers the release mechanism. The containment sheath 58 in FIG. 54 is depicted as no longer constraining the attachment wires 12. This method of attachment and release can be used in holding and then detaching any foreign material such as, but not limited to, plastic, fabric, metal, alloy or any combination in any artery or vein or, indeed, in any tissue where a suture or other attachment device is intended to be a temporary (seconds, minutes, hours, days, months, years) attachment mechanism from which subsequent release may be desired. A similar release mechanism could be achieved with any object that changes character when exposed to energy such as light including, but not limited to, infrared and ultraviolet, sound waves, or electricity. Magnets and glues that dissolve upon electrical or heat stimulation or metal alloys that separate in response to heat or electrical current (this includes using resistive heating for activating a heat sensitive release mechanism for any foreign body within any vessel (artery or vein) or within any tissue space can also be used).

FIG. 55 depicts the attachment wires 12 captured within the containment sheath 58 and compressed toward the catheter 59. The constraining and releasing of the attachment wires illustrated is achieved by a push/pull mechanism. This could also be performed by a screwing mechanism, a ratcheting mechanism, or a twisting or turning mechanism.

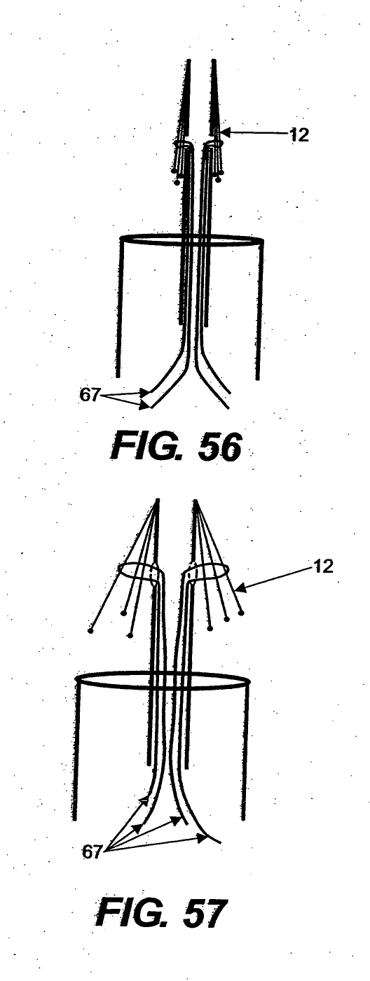

FIGS. 56 and 57 depict another embodiment to constrain (FIG. 56) and release (FIG. 57) the attachment wires 12 with pull wires 67.

In an alternate embodiment, FIG. 58 depicts a method of attaching and detaching the attachment wires 12 to the tube portion 2 of the graft. The graft is constructed with a semi-rigid circumferential attachment hoop 72 in the neck of the graft 1. The attachment hoop 72 may be composed of, but is not limited to, metal or polymer, or any other suitable material. The hoop serves as an attachment point 73 where the attachment wires 12 connect to splay or contract the graft 1. The attachment wires 12 fasten to the attachment hoop 72 at intervals around the hoop either by the inserter or at the factory before sterilization. The attachment wires 12 may be, but are not limited to, circumferential struts. The struts can be normally splayed as shown, but is not limited to, a diameter of about 10-30 mm or they can be normally axial with the support wire 11 and activated with secondary struts as is familiar in an umbrella opening mechanism. Schemes relating to reversible attaching and detaching are exemplary only and are not limited to those described below.

In one scheme, the attachment hoop is withdrawn from the strut, graft and the patient by an endovascular grasper allowing the struts to assume a position axial with the central catheter.

In a second scheme, the attachment hoop is a polymer that dissolves in blood in a time frame sufficient to carry out the correct placement and fastener attachment.

In a third scheme the attachment point detaches given sufficient force or energy which may include, but is not limited to electrical energy. With electrical energy the attachment point can soften and separate at the attachment points thus releasing the struts and the support wire. With electrical energy the strut end (attachment point) can assume a shape that releases the circumferential hoop.

It should be noted that the attachment hoop is not necessarily linear and can be shaped to be folded and inserted or can be shaped at the attachment points. Additionally, the attachment hoop does not necessarily need to be continuous and can be intermittent and confined to the attachment points.

In a fourth scheme, the attachment hoop is a long flexible strand such as, but is not limited to metal or polymer of a length allowing it to extend to the exterior of the patient. In this scheme the strand is pulled and it releases the struts at the attachment points.

FIG. 59 depicts the tube portion 2 of an graft contained within the short sheath 25 and the graft limbs 3 and 4 not constrained by the partial sheath portion 63 of the combination sheath. By way of example, FIG. 59 illustrates a graft within a sheath similar to that depicted in FIG. 50.

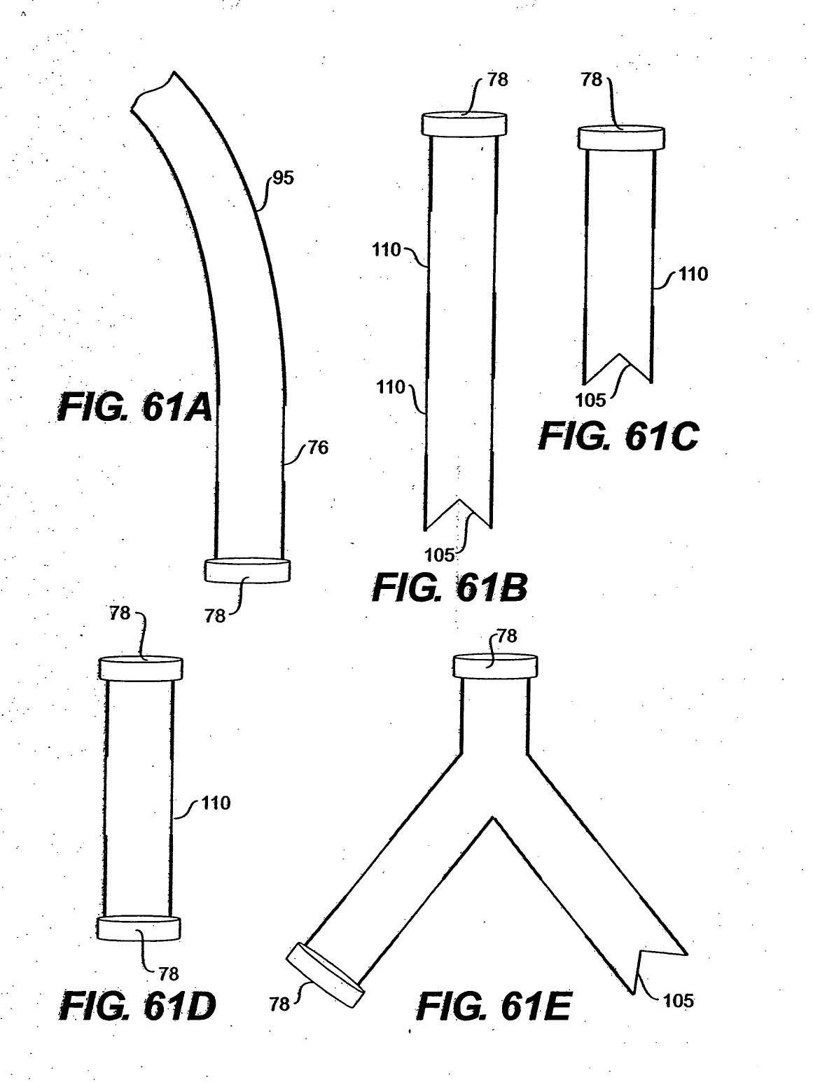

According to FIG. 60, the introducer sheath 95 may be, but is not limited to, about 3 to 30 French (F) (3F=1 mm) in internal diameter. The introducer sheath 95 can have one or more soft or resilient areas 110 that may remain outside the artery or vein and can be clamped or snared to eliminate or reduce backflow of blood. The soft or resilient areas can be constructed of, but is not limited to, rubber, cloth, plastic or any other suitable material that is relatively compressible. A hemostatic valve 105 is also depicted. This valve can be replaced with a connector 78, as illustrated in FIG. 61.

FIG. 61 depicts a variety of combinations of introducer sheaths 95 allowing the ability to construct, at the time of use, an introducer sheath that meets the needs of the individual inserting objects into a vessel, artery or vein. The basic sheath, FIG. 61A, may or may not have a soft area built in. It does have a connector 78 (snap-on, screw, latch etc.) that provides the ability to connect a variety of extensions or additional ports. FIG. 61B depicts an extender that has two soft areas 110 and a hemostatic valve 105. FIG. 61C has only one soft area 110 and a valve 105 whereas FIG. 61D has a soft area and a connector 78 on each end. FIG. 61E offers the possibility of adding multiple ports. These embodiments are some of the possible variations of connectors, soft areas and hemostatic valves that could be constructed and are intended to be illustrative and exemplary only.

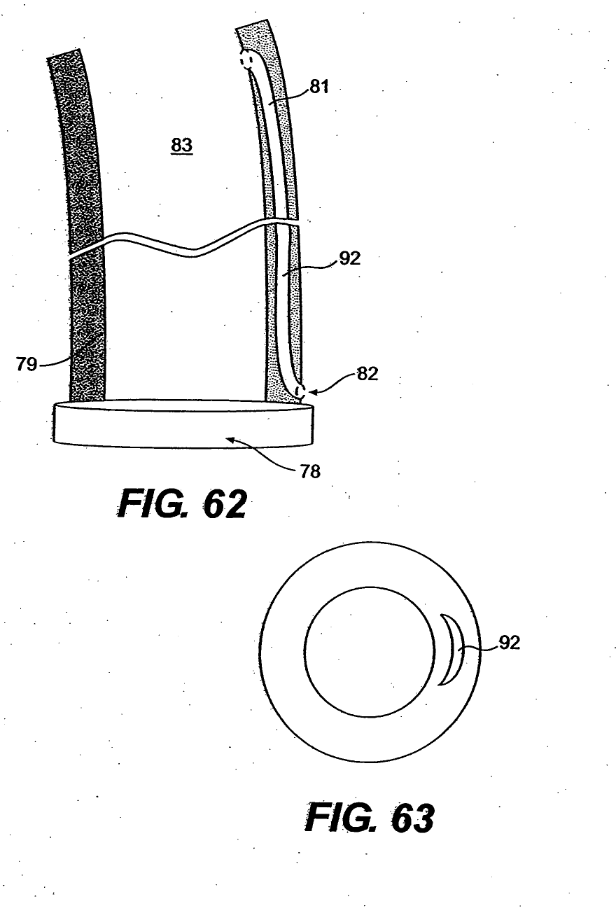

FIG. 62 shows that, within the inner wall 79 of the introducer sheath 95, there can be constructed a small port 81, 82 or multiple ports for the infusion of a liquid such as, but not limited to, a heparin solution. The ports 81, 82 link to a passageway 92 or a plurlity of passageways that may extend the length of the introducer sheath or a predetermined length. The entrance of the port 82 may be near the connector 78 or hemostatic valve 105 outside the area of insertion into the artery or vein. The ports exit site(s) may be near the top of the introducer sheath into the lumen 83 or multiple exit points at discreet places along the length or continuously along the length from proximal to distal. The method of distributing the infusate distally can be via a lumen in the internal diameter, in the wall or on the outside of the sheath wall via a secondary lumen.

The introducer sheath may have a simple sheath with a side infusion port and hemostatic valve or a simple sheath with a side infusion port and snap-on connector or sheath with soft area with infusion port or any combination thereof. FIG. 63 is a cross-section of the introducer sheath showing the infusion port 81.

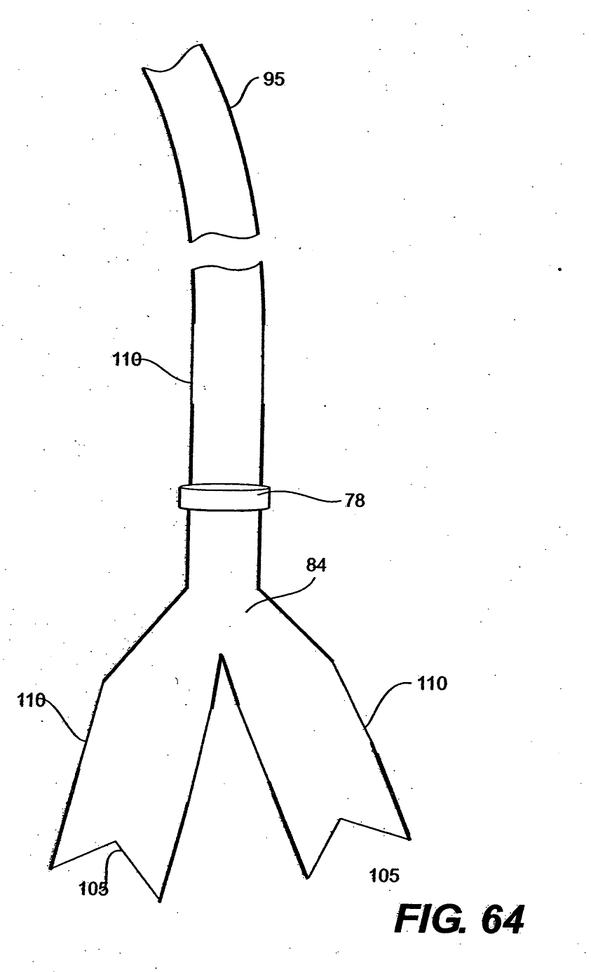

FIG. 64 depicts an introducer sheath 95 containing one soft spot 110 with a connector 78 attached to a double port 84. Each has a soft spot 110 and a hemostatic valve 105. FIG. 64 demonstrates how the connectors work and also shows that the lumens of the add-on ports and extenders can be of varying diameters.

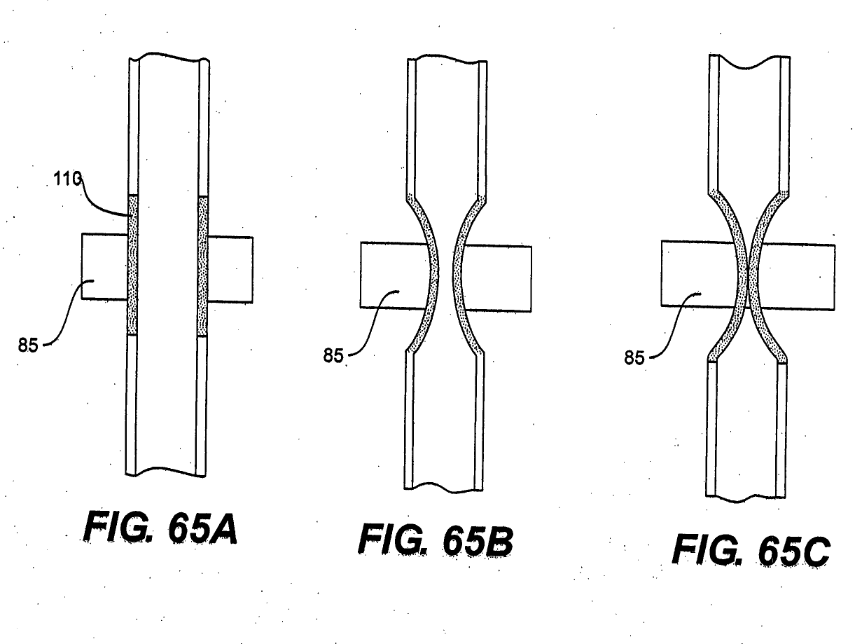

FIG. 65 depicts another possible feature of the introducer sheath 95 with a soft or resilient section 110, also illustrated in FIG. 61. This is a user-controlled variable restricting device 85 that is in circumferential contact with the resilient section 110. The user-controlled variable restricting device 85 is adjustable for the desired internal aperture of the resilient section. The desired internal aperture can be fully open for the largest outer diameter catheter A, partially open for hemostasis around any size catheter (including a guide wire) B, or fully closed for total hemostasis C. The purpose of the device is aperture control and thereby accommodating most sizes of devices passing through the internal diameter of the introducer sheath.

An advantage of alternative embodiments of the invention depicted in FIGS. 66 and 67 is to cut a suture along its length at a location remote from the operator and for the device to track over a suture or along side of it by a suture cutter 80. FIG. 66 shows a catheter based device 80 which tracks over a suture 14 to a remote location and cuts the suture at the distal end of the catheter when the operator inputs mechanical energy into the mechanical actuator 86. In this example the mechanical actuator 86 is pushed and the cutter 87 moves through the suture 14 and cuts it in two pieces 88 and 89. When the mechanical actuator 86 is withdrawn the counter-resistance mechanism 90 such as, but not limited to, spring, flex band as shown, or any other suitable counter-resistance mechanism causes the cutter 87 to return to its original position. Alternatively a spring can act as the force driving the cutter through the suture when the mechanical actuator is withdrawn. Also, the actuator and cutter can be arranged so that rotational or linear movement will cause the cutter to pass through the suture and cut it. An alternative method of affecting the cut of the suture can be laser light. The source of laser light immediately adjacent to the suture would be an optical fiber or a laser diode.

It is further noted that the suture can be fed through the lumen of the suture cutter via a simple snare. If the suture cutter tracks along side of the suture, the suture can be fed through a shorter lumen either axially or from the side. A side loading design would allow the suture to enter from the side but not allow its exit during the tracking procedure prior to cutting.

It will be apparent to those skilled in the art that variations and modifications of the present invention can be made without departing from the scope or spirit of the invention. For example, the method of performing a surgical procedure could be used in settings other than the repair of aneurysms. The method could be used to attach any prosthetic material to any tissue with a metal or plastic attachment device, such as a shape memory metal, plastic staple, or metal staple. For instance, the method could be used to attach a prosthetic mesh to fascia through a laparoscope/endoscope or directly in an open operation for hernia repair. Thus, it is intended that the present invention cover all such modifications and variations of the invention, provided they come within the scope of the appended claims and their equivalents.

The following descriptions of the preferred embodiments of the present invention are described, for purpose of example, in connection with the repair of an abdominal aortic aneurysm. The inventors of the present subject matter contemplate that the embodiments described herein are capable of use in the repair of other vessels and in other procedures. Thus, it is intended that the present invention cover the modifications and variations of the invention, provided they come within the scope of the appended claims and their equivalents.

### Repair Graft

Reference will now be made in detail to preferred embodiments of grafts according to the present invention for repair of abdominal aortic aneurysms, an example of which is illustrated in FIGS. 68-77.

FIGS. 68A and 69A depict a preferred embodiment of the repair graft assembly of the present invention directed to a proximal graft assembly 10000 and distal graft assembly 20000 for repair of a vessel 1000. The proximal graft assembly 10000 and distal graft assembly 20000 are secured to a wall 2000 of the vessel 1000 to exclude the aneurysm from the circulatory system of the patient. In the preferred embodiment of the present invention, the proximal graft assembly 10000 is a bifurcated tube graft.

The distal graft assembly 20000 preferably comprises an attachment cuff 21000. The attachment cuff 21000 is sized to secure the distal graft assembly 20000 to the wall 2000 of the vessel 1000 at the distal end of the vessel 1000. The distal graft assembly 20000 also comprises at least one graft attachment leg, tube or branch 22000. The attachment cuff 21000 is secured to the wall 2000 of the vessel 1000 out to the adventitia using a suitable fastener, described in detail below.

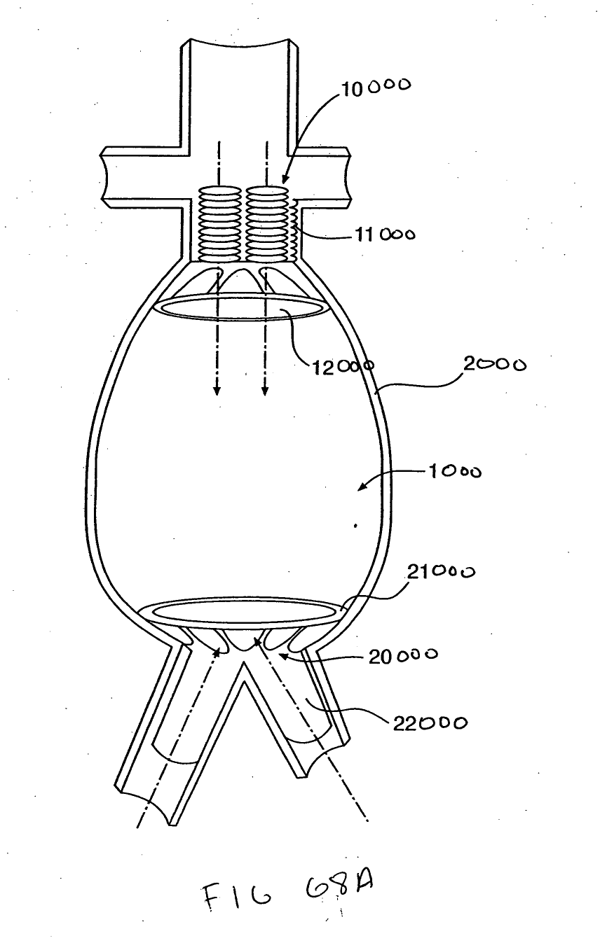

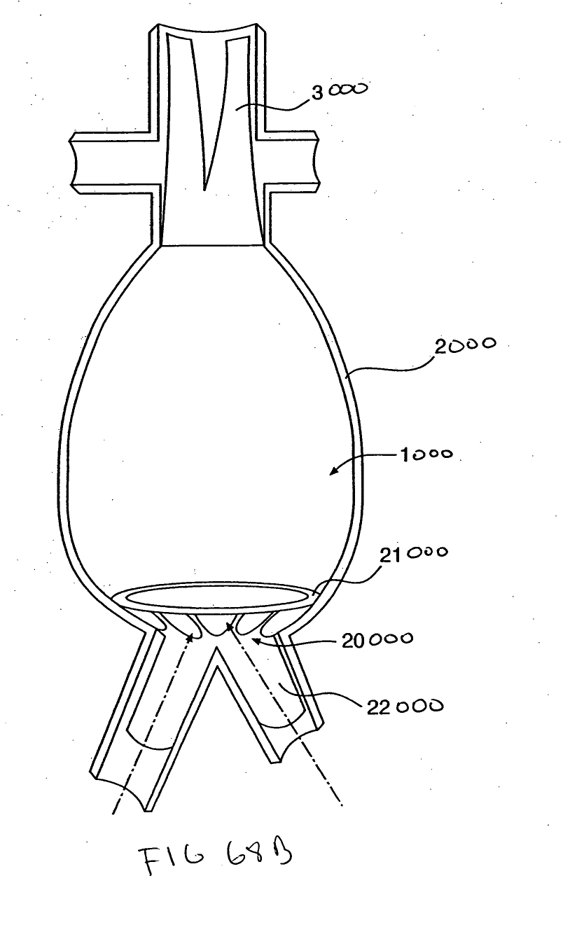

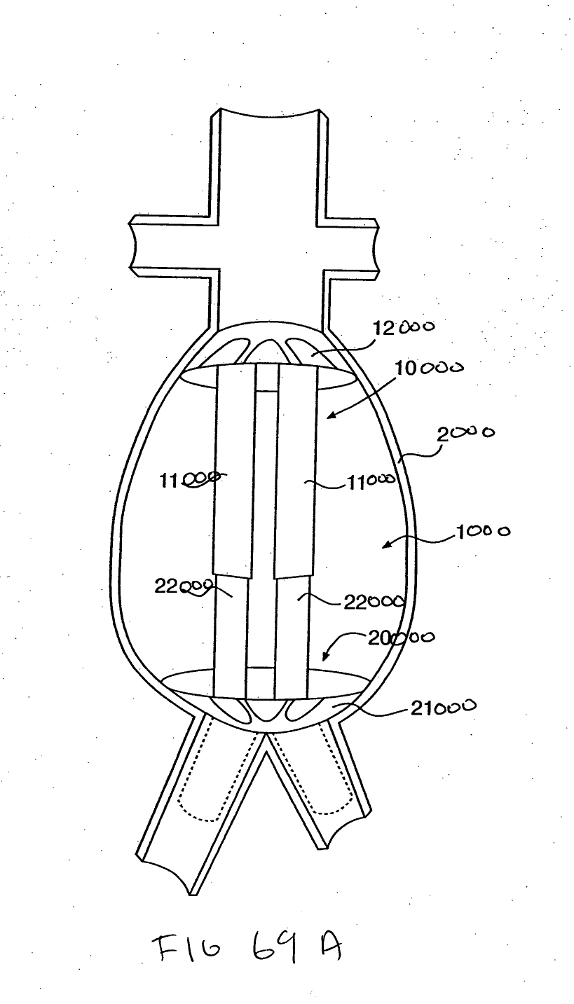

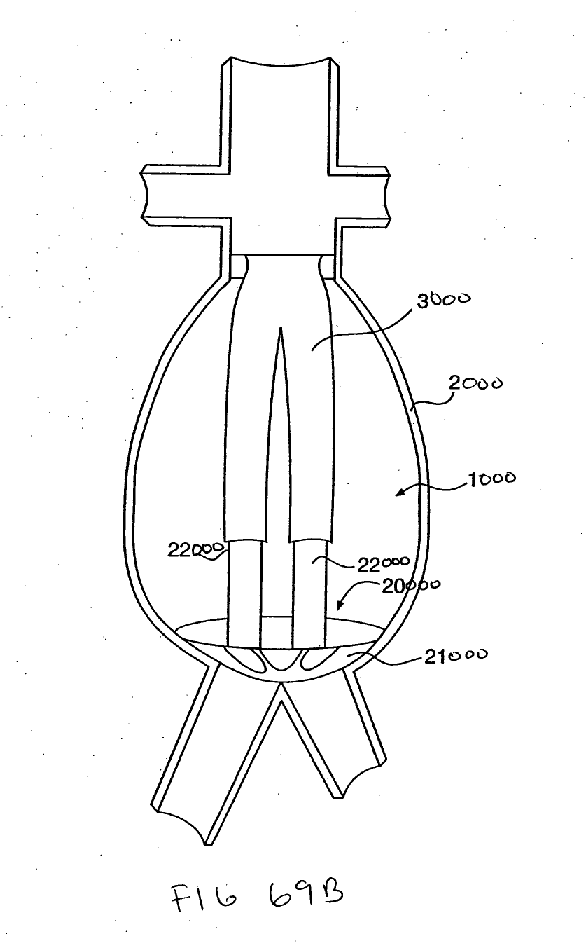

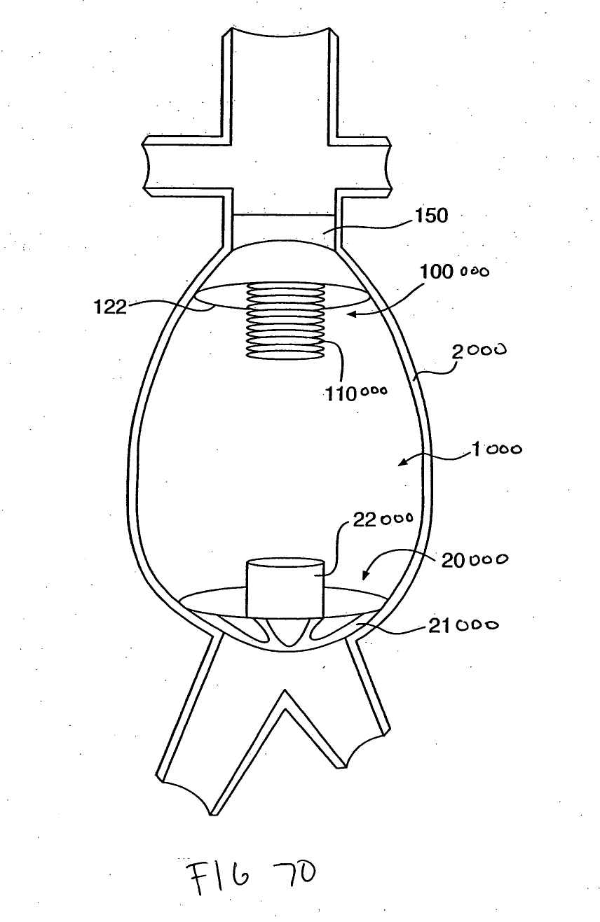

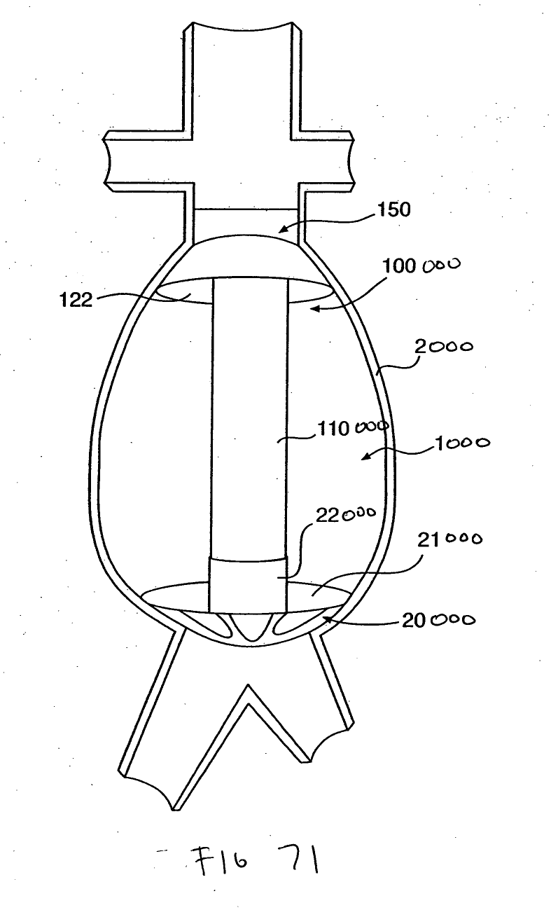

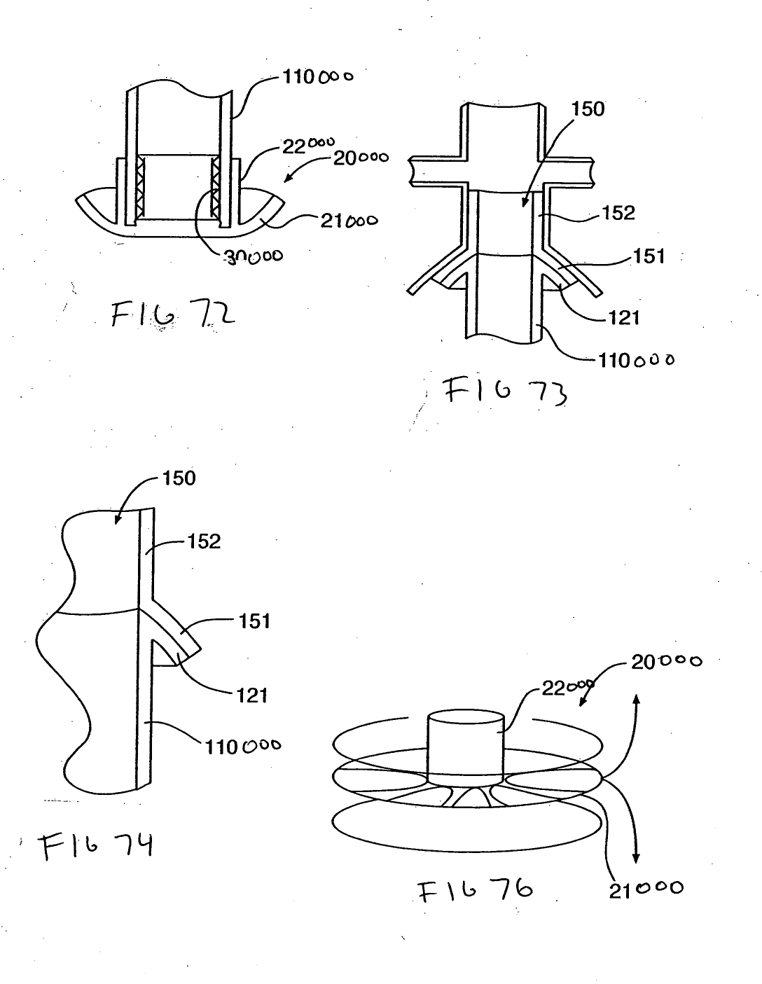

The distal graft assembly 20000 is positioned within the distal end of the vessel 1000, as shown in FIG. 68A using a guide wire, not shown, that extends between and through both common iliacs. The attachment cuff 21000 is then secured to distal end of the vessel 1000 out to the adventitia using a repair apparatus, described below. After the attachment cuff 21000 is firmly secured to the wall 2000, attachment tubes 22000 are invaginated to the position shown in FIG. 69A. A proximal graft assembly 10000 is then secured to the attachment legs 22000 using suitable connectors, such as, a self-expanding stent 30000000, as shown in FIG. 72.

The bifurcated proximal graft assembly 10000 comprises a pair of tubular legs 11000. The tubular legs 11000 are sized to be received within/without the graft attachment tubes 22000. The bifurcated proximal graft assembly 10000 may also comprise an attachment cuff 12000 for attachment to the wall 2000 of the vessel 1000. The attachment cuff 12000 has a similar structure to the attachment cuff 21000 of attachment device 20000. The tubular legs 11000 are invaginated following the process of securing the attachment cuff 12000 to the wall 2000. The attachment legs 22000 may be positioned within the tubular legs 11000, as shown in FIG. 69A. Alternatively, the tubular legs 11000 may be positioned within the attachment legs 22000, as shown in the embodiment of FIG. 71.

It is also contemplated that the distal graft assembly 20000 may be used with a standard tube graft 3000, as shown in FIGS. 68B and 69B. In this variation, the tube graft 3000 is secured to the wall 2000 of the vessel 1000 while in an inverted position, as shown in FIG. 68B using fasteners, described below, and a self-expanding stent 30000000, if desired. The tube graft 3000 is then invaginated and secured to the distal graft assembly 20000, as described above. The benefit of the invagination of the graft 3000 is that the fasteners securing the graft 3000 to the vessel 1000 are not in direct contact with the blood within the vessel 1000. This will reduce the possible build up of thrombus at the point of attachment and thereafater the creation of emboli.

The proximal graft assembly 10000 and distal graft assembly 20000 will enable the creation of a cross sectional area ratio between the common iliacs and the distal aorta that exists only at childhood. The ratio may be 1.1 to 1.0. This ratio minimizes the reflected wave that is instrumental in the creation of plaque deposits at the distal bifurcation.

FIGS. 70 and 71 depict another embodiment of a repair graft for repair of an abdominal aortic aneurysm 1000 according to the present invention. The proximal graft assembly 100000 is secured to a wall 2000 of the abdominal aorta to exclude the aneurysm 1000 from the circulatory system of the patient. The proximal graft assembly 100000 is used in connection with the distal graft assembly 20000, described above. In this embodiment, the distal graft assembly 20000 comprises a single attachment leg or tube 22000. The proximal graft assembly 100000 comprises a tube graft assembly 110000 for forming a passageway within the vessel 1000.

The radially extending attachment cuff 121 provides a greater surface area for securing the proximal graft assembly 100000 to the wall 2000. Additionally, the radially extending portion 121 is flexible, which permits some positioning adjustment of the proximal graft assembly 100000 in the event the size of the passageway within the abdominal aorta changes after the surgical procedure. FIG. 77 illustrates the flexibility of the attachment cuff 2100 which is similar to attachment cuff 121. Like the embodiment of FIGS. 68A and 69A, the proximal graft assembly 100000 is secured to the vessel wall 2000 in an invaginated manner, as shown in FIG. 70. After the attachment cuff 121 is secured to the vessel wall 2000, the proximal graft assembly 100000 is invaginated to the position shown in FIG. 71. The tubular leg assembly 110000 is then secured to the distal graft assembly 20000, as shown in FIG. 72. In a preferred embodiment, a self-expanding stent 30000 is used to secure it to the attachment leg 22000 of the distal graft assembly 20000. The self-expanding stent 30000 applies radial pressure against an inner surface of tube graft assembly 110000 to secure the tube graft assembly 110000 to the distal graft assembly 20000.

The self-expanding stent 30000 is a preferred method of securing the proximal tube assemblies 10000 or 100000 to the distal graft assembly 2000. However, it will be apparent to those skilled in the art that various modifications and variations can be made in the construction and configuration of the present invention without departing from the scope or spirit of the invention. For example, surgical staples, sutures, adhesives or other methods may be used to secure the proximal graft assembly 10000 to the distal graft assembly 2000. Thus, it is intended that the present invention cover the modifications and variations of the invention, provided they fall within the scope of the appended claims and their equivalents.

As described above in connection with FIGS. 68B and 69B, it is also contemplated that the distal graft assembly 2000 may be used with a standard tube graft, not shown. The tube graft will also be secured to the wall 2000 of the vessel 1000 while in a cephalad position using either fastener devices, described below, or a self-expanding stent 30000. The tube graft is then invaginated and secured to the distal graft assembly 2000, as described above.

FIGS. 73 and 74 depict a proximal attachment assembly 150 according to the present invention for securing the proximal graft assembly 10000 or 100000 to the proximal end of the vessel 1000. It is preferred that the proximal attachment assembly 150 be used in connection with securing the proximal graft assemblies 10000 or 100000 to the vessel wall 2000 according to preferred embodiments of the present invention as shown, for example, in FIGS. 70, 71, 73 and 74. The proximal attachment assembly 150 comprises a cuff attachment portion 151 and a vessel attachment portion 152. The attachment cuff 1200 or 121 is secured to the cuff attachment portion 151, by sewing, for example. The vessel attachment portion 151 is then secured to the vessel 1000 using, for example, a fastener or a self-expanding stent 30000 and fasteners, if necessary. Alternatively, the proximal attachment assembly 150 may be invaginated and secured to the vessel 1000 in the manner described above in connection with FIGS. 68B and 2B. The cuff attachment portion 151 and the attachment cuff 12000 or 121 interact in a manner such that the proximal graft assembly 10000 or 100000 are not impacted by the expansion of the neck of vessel 1000 after the surgical procedure.

Another embodiment of the repair grafts according to the present invention are disclosed in FIGS. 75 and 77. The embodiment of FIGS. 75 and 77 utilizes a pair of distal graft assemblies 20000, which are secured at the proximal and distal ends of the vessel. A proximal graft assembly 10001, which forms a passageway within the vessel 1000 interconnects the distal graft assemblies 20000. As described above, the proximal graft assembly 1000 is secured to the attachment legs 22000 of the distal graft assemblies 20000 using a self-expanding stent 30000 or other suitable fastening means. The attachment legs 22000 may be inserted in the proximal graft assembly 1000. Alternatively, the proximal graft assembly 1000 may be inserted in the attachment legs 22000, as shown in FIG. 78.

The above described repair grafts facilitates repair of a vessel in a manner that is neither profile nor dimension dependent. This is especially helpful in view of the fact that the necks of the post-surgical aorta typically increases in size for approximately twelve months. The above-described repair grafts accommodate such expansion without allowing leaks or graft migration. The attachment cuffs are capable of accommodating dimensional changes in the necks of the abdominal aorta. Furthermore, the use of the distal graft assembly 2000 permits distal attachment removing the need for iliac/femoral attachment.

In the above described embodiments, the proximal graft assemblies 10000, 100000 and 1000, distal graft assembly, and proximal attachment assembly 150 are preferably formed from Gore-Tex® or equivalent biocompatible material. It will be apparent to those skilled in the art that various modifications and variations can be made in the construction and configuration of the present invention without departing from the scope or spirit of the invention. For example, in the embodiments mentioned above, various other suitable materials such as, Dacron®, and other biocompatible graft materials may be used to form the repair grafts. Thus, it is intended that the present invention cover the modifications and variations of the invention, provided they fall within the scope of the appended claims and their equivalents.

Similar to other graft procedures, the proximal graft assemblies 10000, 100000, or 1000000 according to the present invention require attachment to the wall 2000 of the vessel. Often, it is necessary to attach the distal end of the graft into material which is routinely calcified and therefore difficult to penetrate. When paired with the absence of a distal neck in the vessel, the presence of the plaque has forced others to promote the use of a bifurcated graft in which the graft limbs are fastened by stents within the common iliac or femoral arteries. This procedure may potentially damage the femoral arteries. Furthermore, the presence of a graft and stent within the iliac or femoral arteries potentially restricts the flow of blood within the vessels. This is unnecessary when utilizing the repair grafts according to the present invention.

### IntraVascular Endoscopy (IVE) Based Repair System

Reference will now be made in detail to preferred embodiments of an apparatus according to the present invention for facilitating the repair of abdominal aortic aneurysms using above described grafts. An example of an intravascular endoscopy based system is depicted in FIGS. 78-84.

The repair apparatus 5000 comprises a housing 200 for alternately receiving a visualization apparatus 6000 and a penetration apparatus 7000, as shown in FIG. 82D. It, however, is contemplated by the inventors of the present invention that the visualization apparatus 6000 and penetration apparatus 7000 may be combined into a single assembly within the repair apparatus 5000. The housing 200 has a hollow construction, as illustrated in FIG. 79, which permits insertion of the visualization apparatus 6000 or the penetration apparatus 7000, described in detail below. The housing 200 is divided into two primary portions: static housing portion 210; and flexible housing portion 220. The housing 200 has a sufficient length such that it extends from the repair site within the vessel 1000 through the appropriate or chosen artery to a point outside the patient.

The housing 200 has a hollow interior 211 to permit passage of one of the interchangeable apparatus 6000 and 7000. An inner surface of the hollow interior 211 comprises rotation prevention means 212 for properly orienting the interchangeable apparatus 6000 and 7000 within the housing 200. In a preferred embodiment, the rotation prevention means 212 is a ridge, as shown in FIG. 79, that extends along the inner surface of the hollow interior 211. It, however, will be apparent to those skilled in the art that various modifications and variations can be made in the construction and configuration of the present invention without departing from the scope or spirit of the invention. For example, the rotation prevention means 211 mentioned above, may be located at different radial positions within the housing and may also be a ridge, a groove, a plurality of grooves, or other devices capable of preventing rotation of the interchangeable apparatus 6000 and 7000 within the housing 200. Thus, it is intended that the present invention cover the modifications and variations of the invention, provided they fall within the scope of the appended claims and their equivalents.

Positioned within the housing 200 is an apparatus guide means 214 for guiding the repair apparatus 5000, as shown in FIGS. 78 and 82A, within the vessel 1000 during use. The guide means 214 preferably is a passageway or lumen extending within the housing wall through the static portion 210. A guiding means 160 cooperates with guide means 214 to guide the apparatus 5000 during use. The guiding means 160 is preferably a guide wire which is capable of extending from the femoral artery to the axillary artery. In a preferred embodiment, the guide wire 160 is a filament (e.g., stainless steel, titanium or a Kevlar®). It, however, will be apparent to those skilled in the art that various other materials having similar properties of physical integrity, high strength, flexibility, and minimal thermal expansion may be used to form the guide wire 160. The guide wire 160 projects from the flexible housing portion 220 through an aperture 226 in the housing 200, as shown in FIG. 81.

Housing 200 also comprises an apparatus manipulation means 215 to aid in manipulating and orienting the apparatus 5000 within the vessel 1000 during the repair operation. The manipulation means 215 preferably comprises at least one passageway extending within the housing wall through the static housing portion 210 and terminating in the flexible housing portion 220. A manipulating means 170 cooperates with manipulation means 215 to guide the apparatus 5000 during use. The manipulating means 170 is preferably comprises at least one guide wire that is capable of extending from outside the patient through the housing 200. The guide wires 170 extend through the manipulating means 215. In a preferred embodiment, the guide wires 170 are filaments (e.g., stainless steel, titanium or a Kevlar®). It, however, will be apparent to those skilled in the art that various other materials having similar properties of physical integrity, high strength and flexibility may be used to form the guide wires 170.

The guide wires of the manipulating means 170 terminate within the flexible housing portion 220. Operation of the manipulating means 170 results in the articulation of an end portion of the flexible housing portion 220. The guide wires 170 maintain the flexible housing portion 220 in an articulated position, as shown in FIGS. 78 and 81, such that the visualization apparatus 6000 and the penetration apparatus 7000 can be interchanged without altering the orientation of the repair apparatus 5000 with respect to the surgical site.

The wall of the static housing portion 210 comprises an outer surface formed from silicone and an inner surface formed from Teflon®. It, however, will be apparent to those skilled in the art that various modifications and variations can be made in the construction and configuration of the present invention without departing from the scope or spirit of the invention. For example, the housing wall may be formed from a suitable polymer (e.g., Pebax®) or other material having similar properties including, but not limited to biocompatability, flexural strength, low coefficient of friction. Thus, it is intended that the present invention cover the modifications and variations of the invention, provided they fall within the scope of the appended claims and their equivalents.

The flexible housing portion 220 may be formed in a manner similar to static housing portion 210. For example, the housing may comprise an outer surface formed from silicon and an inner surface formed from Teflon®. It, however, will be apparent to those skilled in the art that various modifications and variations can be made in the construction and configuration of the present invention without departing from the scope or spirit of the invention. For example, the lining may be formed from a suitable polymer or other material having similar properties including, but not limited to biocompatability, flexural strength, low coefficient of friction. Alternatively, the flexible housing portion 220 may comprise a coiled metallic spring outer casing 224 that surrounds a lining. The lining may be formed from Teflon®. The coiled metallic spring outer casing 224 may be formed from a biocompatible stainless steel or titanium. Furthermore, the spring outer casing 224 may be formed from other suitable spring materials. It is not necessary that the outer spring casing 224 extend along the entire length of the flexible housing portion 220. Rather, the outer spring casing 224 may be positioned along the portion of the flexible housing portion 220 that is subject to bending. However, it is contemplated that an outer spring casing that extends along the entire length of the flexible housing portion 220 be within the scope of the present invention.

The flexible housing portion 220 and the static housing portion 210 are manufactured as separate components. It, however, will be apparent to those skilled in the art that various modifications and variations can be made in the construction and configuration of the present invention without departing from the scope or spirit of the invention. For example, the static housing portion 210 and the flexible housing portion 220 may be formed as a single component. In a preferred embodiment, the static housing portion 210 is permanently secured to the flexible housing portion 220. However, it is contemplated that the housing portions 210 and 220 may also be removably attached.

FIGS. 82B and 82C illustrates another repair apparatus 500 for alternatively receiving a visualization apparatus 6000 and a penetration apparatus 7000 according to another embodiment of the present invention. The repair apparatus 500 comprises a housing 20001 for alternatively receiving a visualization apparatus 6000 and a penetration apparatus 7000. The housing 20001 is flexible and has a sufficient length such that it extends from the repair site within the vessel 1000 through the appropriate artery to a point outside the patient.

The housing 20001 is hollow, as described above in connection with housing 200, to permit passage of one of the interchangeable apparatus 6000 or 7000. The housing 20001 includes at least one guide means 2140 positioned at the exterior of the housing 20001 for guiding the repair apparatus 500 within the vessel 1000 during use. The guide means 2140 preferably is a passageway extending along the exterior of the housing wall to a point adjacent the distal end 2001 of the housing 20001.

Guide wires 160 extend within the guide means 2140. The guide wires 160 extend from the end of guide means 2140 and are secured to the distal end 2001 of the housing 20001, as shown in FIGS. 82B and 82C. Adjustment of the guide wires 160 manipulates the position of the repair apparatus 500 within the vessel 1000. The above described arrangement permits a wide range of articulation of the repair apparatus 500 within the vessel 1000.

An additional guide wire 161 is secured to the distal end 2001 of the housing 20001. The guide wire 161 extends through the vessel 1000 and appropriate artery to permit the positional adjustment of the repair apparatus 500 within the vessel.

FIG. 82D illustrates another repair apparatus 5000 for alternatively receiving a visualization apparatus 6000 and a penetration apparatus 7000 according to another embodiment of the present invention. The repair apparatus 5000 comprises a flexible hollow housing 2010 and has a sufficient length such that it extends from the repair site within the vessel 1000 through the appropriate artery to a point outside.

The housing 2010 includes at least one guide wire 162 extending along the exterior of the housing 2010, as shown in FIG. 82D. The housing 2010 includes an inflatable portion 2011, located adjacent the distal end 2001. Inflation of the inflatable portion 2011 permits articulation of the repair apparatus 5000 within the vessel 1000. A passageway, not shown, extends within the housing 2010 to permit inflation of the inflatable portion 2011 with a suitable fluid, such as, saline or suitable liquid polymers or air. An additional guide wire 161 is secured to the distal end 2001 of the housing 2010. The guide wire 161 extends through the vessel 1000 and appropriate artery to permit the positional adjustment of the repair apparatus within the vessel.

The overall dimensions of the repair apparatus 5000 allows axillary access. This previously was not possible. In this regard, the repair apparatus used in connection with the visualization apparatus 6000 or penetration apparatus 7000 is capable of being used in other surgical procedures not previously contemplated. The apparatus size permits insertion through an introducer sheath device 900, described below. The apparatus 5000 may also be introduced into a vessel percutaneously. This procedure is less invasive and/or intrusive when compared to other repair surgical procedures.

### IntraVascular Endoscopy (IVE) Visualization Apparatus

Reference will now be made in detail to preferred embodiments of the interchangeable apparatus 6000 and 7000 for use with the repair apparatus 5000 according to the present invention for facilitating the repair of abdominal aortic aneurysms. The visualization apparatus 6000 will now be described in connection with FIGS. 78 and 80.

A visualization apparatus 6000 may be inserted within the repair apparatus 5000 to illuminate and permit real time direct viewing of the abdominal aorta to aid and the diagnosis and repair of the aneurysm. The visualization apparatus 6000 is an intravascular endoscope based system that comprises a housing 300 for housing various illuminating and viewing components. The housing 300 is preferably formed as a conduit that is sized to slide within housing 200. In a preferred embodiment, the housing 300 is an extrusion of silicon, Teflon® or polymer or other material having similar properties.

The housing 300 extends through the hollow interior 211 of the housing 200. The housing 300 may comprise orientation means 310 for orienting the visualization means 300 within the housing 200. The orientation means 310 cooperates with rotation prevention means 212. In a preferred embodiment, the orientation means 310 is a channel that extends along an outer surface of the housing 300. It, however, will be apparent to those skilled in the art that various modifications and variations can be made in the construction and configuration of the present invention without departing from the scope or spirit of the invention. For example, the orientation means 310 mentioned above may be located at different radial positions within the housing 300. The orientation means 310, may be a ridge, a groove, a plurality of grooves, or other devices that are complementary with the rotation prevention means 212 to prevent rotation of the visualization apparatus 6000 within the housing 200.

As shown in FIG. 80, housing 300 comprises a plurality of passageways 311, 312, 313, 314, and 315 formed therein. The passageways 311, 312, 313, 314, and 315 extend along the entire length of the housing 300. Central passageway 311 is provided for the passage of optical viewing means 320 for viewing an abdominal aorta. In a preferred embodiment, the optical viewing means 320 is a fiber optic system. The system incorporates a fiber optic bundle. It, however, will be apparent to those skilled in the art that various modifications and variations can be made in the construction and configuration of the present invention without departing from the scope or spirit of the invention. For example, the optical viewing means 320 mentioned above, may be any flexible optical system that is sized for use in surgical applications. The optical viewing means 320 permits real time direct viewing of the area of repair in the vessel 1000. The optical viewing means 320 may be connected to a video camera and monitor, not shown, that permits the surgeon to view the repair area The images may be stored and recalled as desired by using either a video printer or video cassette recorder. The penetration apparatus 7000 will be located at the same position as the visualization apparatus 6000. The penetration apparatus 7000 incorporates a radio opaque marker that will indicate the precise position of the penetration apparatus 7000 on the monitor. This allows the surgeon to monitor and track the adjustments of the repair apparatus 5000.

Peripheral illumination passageways 312 and 313 are provided for the passage of illuminating means 330 for illuminating the abdominal aorta for viewing by the optical viewing means 320. In a preferred embodiment, the illuminating means 330 is a fiber optic system including a fiber optic bundle. It, however, will be apparent to those skilled in the art that various modifications and variations can be made in the construction and configuration of the present invention without departing from the scope or spirit of the invention. For example, the illuminating means 330 mentioned above, may be any system that is sized for use in surgical applications and capable of illumination within the aorta. Although a pair of passageways are illustrated, it is contemplated that a single illumination passageway will provide sufficient illumination. Additionally, more than two passageways may also be provided.

Peripheral fluid inflow passageway 314 and peripheral fluid outflow passageway 315 are provided for the passage of fluid lens media to and from the visualization tip 340. The peripheral fluid inflow passageway 314 supplies a stream of optically clear fluid lens media from the visualization tip 340 in the area in front of the optical viewing means 320. A control means, not shown, may be incorporated into passageway 314 to control the flow volume and velocity of the fluid lens media to the visualization tip 340. The control means may be a valve or other suitable flow control devices. The control means controls the optically clear fluid lens media such that blood within the aortic cavity and the fluid lens media are pressure balanced. As a result, blood that is typically within the aorta is temporarily diverted away by the fluid lens media to a point adjacent the area of the wall 2000 to be viewed by the optical viewing means 320. The infusion of fluid lens media will dilute blood to an appropriate transparency in the immediate surgical site to exclude blood between the visualization tip 340 and the surgical site on the wall 2000. This permits the surgeon to clearly view the wall 2000 through the optical viewing means 320. In a preferred embodiment, the fluid lens is a transparent fluid to permit viewing of the wall 2000. The fluid lens media may be a saline solution. It is preferred that the solution be used for a single application (i.e., it is not reused). Other media, such as CO.sub.2 gas and GreenX liquid fluorocarbon are contemplated to be within the scope of the present invention. The peripheral fluid outflow passageway 315 acts as a return duct for the fluid lens media within the aorta. Alternatively, the fluid lens media may then be filtered using an appropriate filtering means and recirculated using a pumping means through the peripheral fluid inflow passageway 314.

In a preferred embodiment, it is contemplated that the visualization apparatus 6000 be used in combination with the introducer sheath devices 900, described below. The introducer sheath devices 900 and in particular the positioning assembies 920 permit the isolation of a portion of the vessel during the repair procedure. Specifically, the positioning assemblies 920 within the common iliacs and femoral artery permit the control of blood within the vessel. With this arrangement, it is then possible to more readily divert blood away from a viewing area with the flow of fluid lens media from the fluid inflow passageway 314.

A visualization tip 340 is securely mounted to the end of housing 300 in a fluid tight manner. The tip 340 may be snap fitted or permanently mounted to the housing 300. It, however, will be apparent to those skilled in the art that various modifications and variations can be made in the construction and configuration of the present invention without departing from the scope or spirit of the invention. For example, the visualization tip 340 mentioned above, may be secured to the housing 300 by means other than the above described snap and permanent fittings. The visualization tip 340 may be formed by injection molding or other suitable manufacturing methods in silicone or similar polymer.

The visualization tip 340 comprises apertures 341, 342, 343, 344, and 345 that correspond to passageways 311, 312, 313, 314, and 315, respectively. Aperture 341 contains a lens positioned therein to facilitate viewing of the wall 2000 with the optical viewing means 320. Apertures 342 and 343 may include windows therein whereby light from the illuminating means 330 passes through the windows to illuminate the wall 2000, although it is not necessary. Apertures 344 and 345 act as gates for the peripheral fluid inflow passageway 314 and peripheral fluid outflow passageway 315. The aperture 344 may be inwardly tapered, such that the inside diameter of the aperture adjacent the inflow passageway 314 is greater than the diameter on the outer surface of the tip 340 to concentrate the stream of fluid lens media from the fluid inflow passageway 314. The aperture 345 may be outwardly tapered, such that the inside diameter of the aperture adjacent the inflow passageway 315 is less than the diameter on the outer surface of the tip 340. It is contemplated that the tip 340 is optional.

### Penetration Apparatus

A penetration apparatus 7000 will now be described in connection with FIGS. 82-87. The penetration apparatus 7000 may be inserted within the repair apparatus 5000, 500, 5000, as shown in FIGS. 82A-D, for fastening a repair graft to the vessel wall 2000. The penetration apparatus 7000 comprises several components for fastening a repair graft including penetration means 420, secondary penetration means 430, tracking means 440 and insertion means 450. The penetration apparatus 7000 comprises housing 410 for housing the penetration means 420, secondary penetration means 430, tracking means 440 and insertion means 450. In a preferred embodiment, the housing 410 has a thin walled tri-limbed profile, as shown in FIGS. 82C, 83, and 84. In a preferred embodiment for increased flexibility, the housing 410 is positioned within the repair apparatus 5000 such that two of the three limbs of the housing 410 are spaced from the side of housing 200 containing the guide wire 160. It, however, will be apparent to those skilled in the art that various modifications and variations can be made in the construction and configuration of the present invention without departing from the scope or spirit of the invention. For example, the housing 410 mentioned above, may have more than three limbs. Alternatively, the housing 410 may be cylindrical having a plurality of inwardly projecting limbs. An alternative configuration for housing 4100 is depicted in FIGS. 82B and 15D. The housing 4100 comprises a central passageway 4110 containing penetration means 420. Additional passageways 4210 and 4130 are provided for other components such as secondary penetration means, tracking means and insertion means.

The housing 410 is preferably formed from an extrusion of silicone, Teflon®, or polymer having similar properties. Housing 410 comprises a plurality of passageways 411, 412, 413, and 414, formed therein as shown in FIG. 83. An alternative arrangement is shown in FIG. 84. The passageways 411, 412, 413, and 414 extend along the entire length of the housing means 410. Primary passageway 411 is provided for the passage of the penetration means 420. The penetration means 420 is provided to create a treatment specific hole in the wall 2000 of the abdominal aorta for securing the graft thereto with a suitable fastener device, described below. The penetration means 420 penetrates the potentially calcified vessel wall 2000 to securely fasten the repair graft to the wall 2000. The penetration means 420 may be either a laser penetrating device or a piezoelectric penetrating device. It, however, is contemplated by the inventors of the present invention that other penetration means including but not limited to CO.sub.2 penetration, micro electromechanical systems, and intraluminal suturing are considered to be within the scope of the present invention. The laser penetrating device 420 preferably is an IR fiber optic based system using laser energy to create treatment specific holes in the aorta wall 2000. The fused silica/quartz fibers that are utilized are in the 200-600 micron size range. Suitable lasers comprise but are not limited to an acousto optical laser having a wavelength of about 1.35 mu.m, and a Holmium-Yag laser having a wavelength of about 2.1 mu.m. The selected wavelength allows transition of laser energy through the fiber in the passageway 411. The laser fiber will be in direct contact with the surgical site such that the fiber projects from the end of the housing 410. It is contemplated that a single, or tri-pronged hole pattern will be created using penetration means 420 and secondary penetration means 430.

The piezoelectric penetrating device preferably is a catheter based system, which utilizes acoustic vibrations to create treatment specific suture holes to aid in graft/tissue attachment. The piezoelectric penetrating device applies an “acoustic wave” effect to create holes in the graft and vessel wall. In this variation, the passageway 411 preferably contains a super elastic titanium catheter, in rod or tube form, which enables transmittance of energy through the sometimes tortuous vessels to the surgical site. The catheter will be in direct contact with the surgical site such that the catheter projects from the end of the housing 410 into the formed treatment specific hole. The secondary penetration means 430 creates one or more temporary hole(s). The piezo-electronic device preferably operates at a frequency of 20 KHz. Other frequencys, both higher and lower, are contemplated to be within the scope of the present invention. The primary penetration means 420 is coaxial with the fastener devices such that the fastener devices may be inserted through the treatment specific hole created by the primary penetration means 420.

Secondary passageway 412 is provided for the passage of the secondary penetration means 430. The secondary penetration means 430 is also provided to create one or more temporary holes in the vessel wall 2000, in a manner similar to the primary penetration means 420. Similarly, the secondary penetration means 430 may be either a laser penetrating device or a piezoelectric penetrating device, as described above in connection with the penetration means 420. The secondary penetration means 430 serves to anchor and orient the penetration apparatus 7000 while a fastener is inserted within the treatment specific hole formed by the primary penetration means 420. After the secondary penetration means 430 is removed, the temporary holes will seal with blood that will coagulate.

Passageway 413 is provided within the housing 410 for passage of the insertion means 450, described below. Passageway 414 is provided within the housing means 410 for passage of the tracking means 440. In a preferred embodiment, the tracking means 440 is a radiopaque marker, which is utilized for the purpose of identifying the location of the penetration apparatus 7000 within the image on the monitor. It, however, will be apparent to those skilled in the art that various modifications and variations can be made in the construction and configuration of the present invention without departing from the scope or spirit of the invention. For example, the tracking means 440 mentioned above, may be a tip-tracking device or a fiber optic aiming beam.

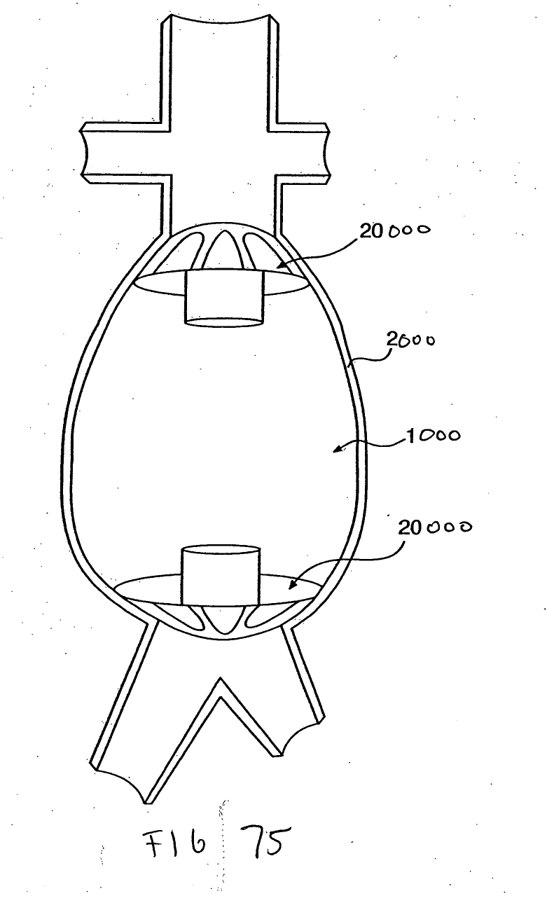

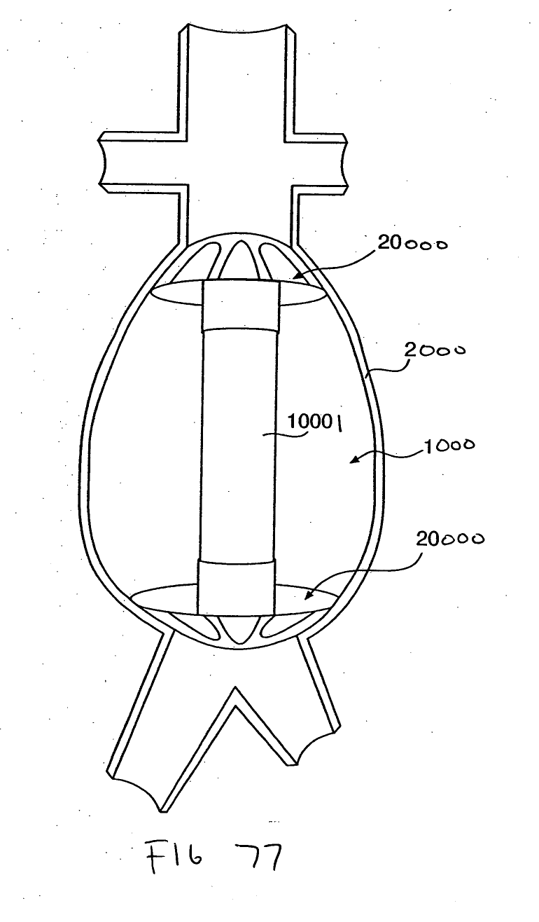

Insertion means 450 for securing the repair graft to the wall 2000 during repair of the aneurysm will be described in connection with FIG. 86. The insertion means 450 preferably comprises a mechanism that drives an individual fastener from a fastener cartridge 460, shown in FIGS. 82 and 85, into and through the treatment specific holes created by the penetration means 420 in the repair graft and wall 2000. The fastener cartridge 460 is capable of holding a plurality of fasteners such that more than one fastener may be sequentially displaced from the cartridge 460 to secure the repair graft to the abdominal aorta wall 2000. Fastener cartridge 460 is preferably detachably connected to housing 410. The fastener cartridge 460 is a hollow housing, as shown in FIG. 85, preferably formed of injection molding HDPE or Liquid Crystal, manufactured by the RTP Co. of MN. The penetration means 420 and 430, the tracking means 440 and the insertion means 450 are appropriately accommodated within the interior of the cartridge structure 460. The cartridge 460 is positioned about the housing 410.

The insertion means 450 illustrated in FIG. 86 comprises a driving means 451 for driving the fastener devices to secure the repair graft to the vessel wall 2000. A gear 452 and fastener advancing means 453 are positioned within an opening 454 in housing 410. In a preferred embodiment, the gear 452 is a worm gear. However, other suitable gear assemblies are contemplated to be within the scope of the present invention. The gear 452 is connected to the driving means 451. The fastener advancing means 453 interacts with the gear 452 to advance a fastener device to secure the repair graft to the vessel wall 2000. In a preferred embodiment, the fastener advancing means 453 is an internally geared drive plate assembly. The drive plate assembly may be capable of limited angular adjustment. Operation of the insertion means 450 is controlled by a control device, not shown, such that upon actuation by the control device, the fastener advancing means 453 is advanced to eject a fastener device from fastener cartridge 460. Alternatively, the insertion means 450 may be hand operated. The insertion means 450 is used, for example, in the embodiment illustrated in FIG. 82C.

Another embodiment of the insertion means 4500 is illustrated in FIG. 87. An insertion cartridge 4510 is secured to the distal end of the repair apparatus 5000. The insertion cartridge 4510 may be snap fitted to the housing 200. The insertion cartridge 4510 comprises a cavity 4511. A spring means 4520 is positioned within the cavity 4511. A fastener cartridge 460 is also located within the cavity 4511. An opening 4530 is located at one end of the insertion housing 4510. The housing 410 of the penetration apparatus 7000 normally prevents the spring means 4520 from ejecting a fastener device through the opening 4530. The insertion means 4500 comprises retraction means 4540 which retracts the housing 410 away from the opening 4530 which permits the fastener to be ejected into the treatment specific hole created by the primary penetration means 420. The retraction means 4540 may be a cable that acts to retract the housing 410 away from opening 4530. The release of the retraction means 4540 causes the housing 410 to return to the position adjacent the opening 4530 to prevent the discharge of a subsequent fastener device.

### IntraVascular UltraSound (IVUS) Repair System

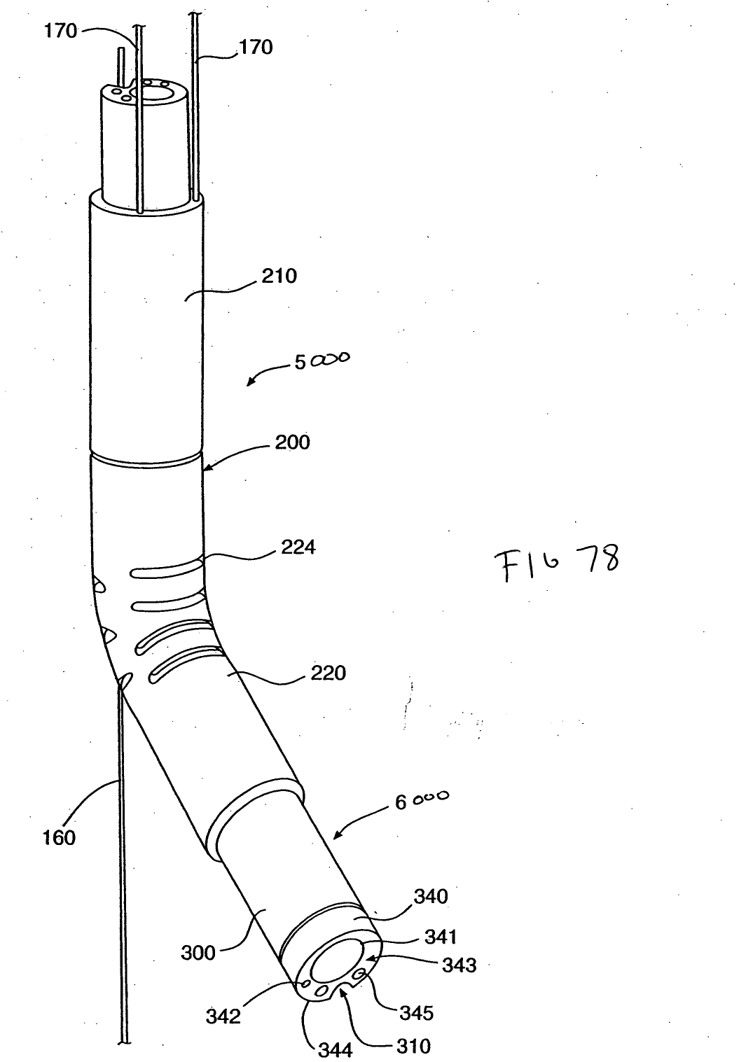

Reference will now be made in detail to preferred embodiments of an apparatus according to the present invention for facilitating the repair of abdominal aortic aneurysms using above described repair grafts. An example of an intravascular ultrasound based system is depicted in FIGS. 88-91.

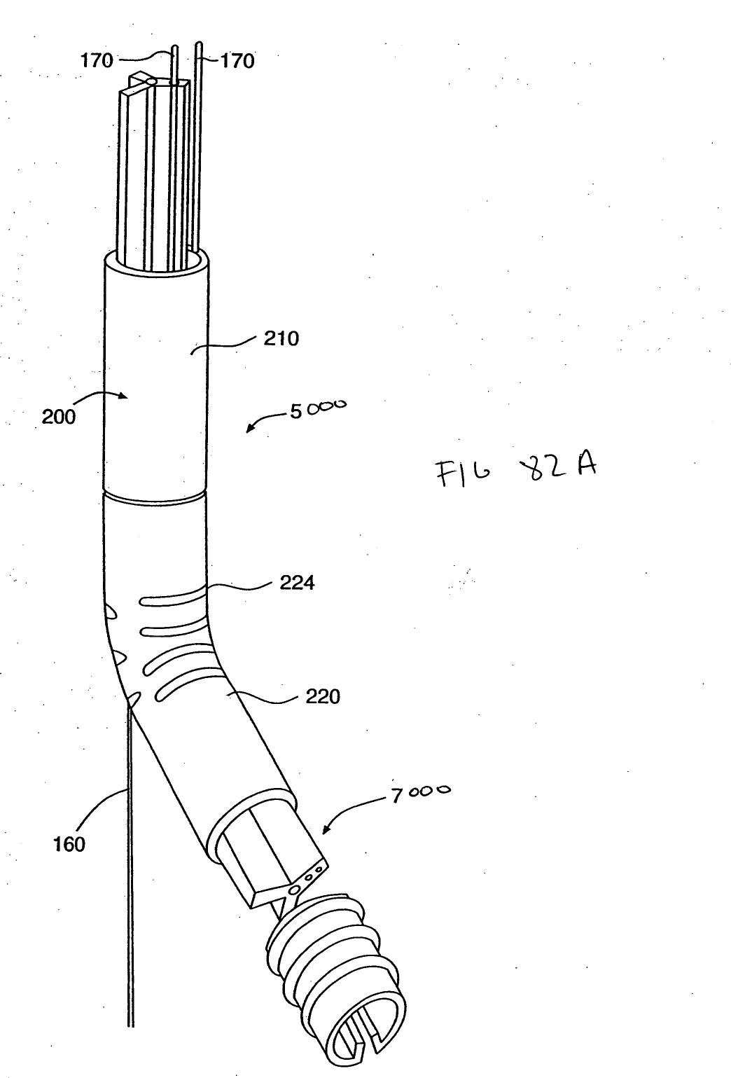

The repair apparatus 50000 comprises housing 800. The housing 800 comprises a major guide wire portion 810, a cross-section of which is shown in FIG. 90, a spacer portion 820, and a minor guide wire portion 830.

Positioned within the housing 800 is an apparatus guide means 214 for guiding the repair apparatus 50000 within the vessel 1000 during use. The guide means 214 preferably is a passageway or lumen extending the length of the housing 800 through major guide wire portion 810, the spacer portion 820, and the minor guide wire portion 830. A guiding means 160 cooperates with guide means 214 to guide the apparatus 50000 during use. The guiding means 160 is preferably a guide wire which is capable of extending from the femoral artery to the axillary artery. In a preferred embodiment, the guide wire 160 is a filament (e.g., stainless steel, titanium or Kevlar® cable). It, however, will be apparent to those skilled in the art that various other materials having similar properties of physical integrity, high strength, flexibility, and minimal thermal expansion may be used to form the guide wire 160.

Housing 800 also comprises an apparatus manipulation means 215 to aid in manipulating and orienting the penetration apparatus 700 within the vessel 1000 during the repair operation. The manipulation means 215 preferably comprises at least one passageway extending within the housing 810. The manipulation means 215 mates with complimentary passageways formed in housing 710. A manipulating means 170 cooperates with manipulation means 215 to guide the apparatus 50000 during use. The manipulating means 170 is preferably comprises at least one guide wire that is capable of extending from outside the patient through the housings 810 and 710. The guide wire 170 extends through the manipulating means 215. In a preferred embodiment, the guide wire 170 is a super elastic metal filament. It, however, will be apparent to those skilled in the art that various other materials having similar properties of physical integrity, high strength and flexibility may be used to form the guide wire 170.

Operation of the manipulating means 170 results in the articulation of an end portion of the housing 710. The guide wire 170 maintains the housing 710 in an articulated position, as shown in FIG. 88, during the repair operation.

The penetration apparatus 700 will now be described in connection with FIGS. 88-91. The penetration apparatus 700 comprises several components for fastening a repair graft including penetration means 420, secondary penetration means 430, tracking means 440, and insertion means 450. The penetration apparatus 700 comprises housing 710 for housing the penetration means 420, secondary penetration means 430, and insertion means 450. In a preferred embodiment, the housing 410 has a thin walled tri-limbed profile, as shown in FIGS. 88, 89 and 91. It, however, will be apparent to those skilled in the art that various modifications and variations can be made in the construction and configuration of the present invention without departing from the scope or spirit of the invention.

The housing 710 is preferably formed from an extrusion of silicone, Teflon®, or polymer having similar properties. Housing 710 comprises a plurality of passageways 711, 712, 713, 714, and 715 formed therein as shown in FIG. 91. The passageways 711, 712, 713, 714 and 715 extend along the entire length of the housing 710. Primary passageway 711 is provided for the passage of the penetration means 420. The penetration means 420 is provided to create an treatment specific hole in the wall 2000 of the abdominal aorta for securing the graft thereto with a suitable fastener device. The penetration means 420 penetrates the calcified portions of the wall 2000 to securely fasten the repair graft to the wall 2000 in the same manner as described above in connection with the endoscopic based system. The penetration means 420 may be either a laser penetrating device or a piezoelectric penetrating device.

Secondary passageway 712 is provided for the passage of the secondary penetration means 430. The secondary penetration means 430 is also provided to create one or more openings in the vessel wall 2000, in a manner similar to the primary penetration means 420, as described above.

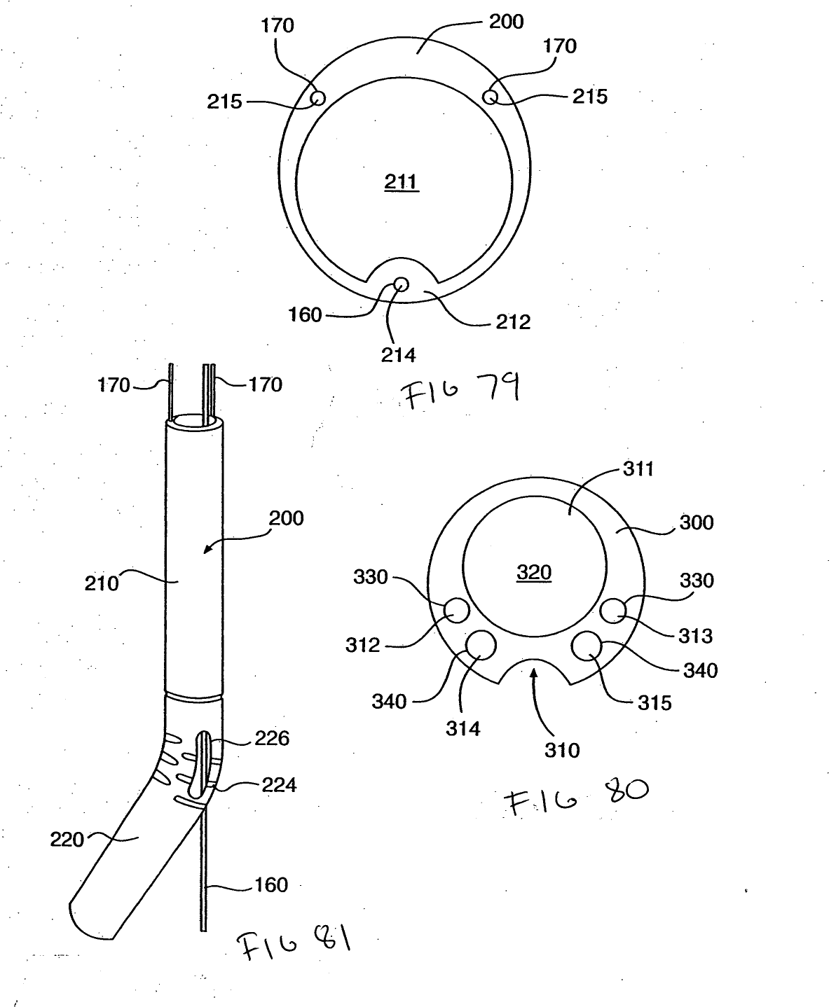

Passageway 713 is provided within the housing 710 for passage of the insertion means 450. Passageway 714 is provided within the housing 710 for passage of the guide wire 170. Passageway 715 is provided for tracking means 440. The insertion means 450 preferably comprises a mechanism that drives an individual fastener from a fastener cartridge 470, shown in FIGS. 88 and 89, into and through the treatment specific holes created by the penetration means 420 in the repair graft and wall 2000. The fastener cartridge 470 is capable of holding a procedure specific quantity of fasteners such that more than one fastener device may be sequentially displaced from the cartridge 470 to secure the repair graft to the wall 2000. Fastener cartridge 470 is preferably detachably assembled to housing 710. The fastener cartridge 470 has a hollow housing 471, as shown in FIG. 88. The penetration means 420 and 430, and the placement/fastener means 450 are appropriately accommodated within the interior of the cartridge structure 460. The cartridge structure 470 and associated fastener device are complimentary with the spacer portion 820 of the housing 800 such that the penetration apparatus 700 has a flush profile, as shown in FIG. 89.

A visualization apparatus 600 for viewing the abdominal aorta to repair the aneurysm is positioned within housing 800 adjacent the minor guide wire portion 830. The visualization apparatus 600 is an intravascular ultrasound (IVUS) based system produced, for example, by Endosonics of Rancho Cordova, Calif., that comprises a housing 601 for housing radial scanning components. The housing 601 may comprise a scanning window 602, however, it is not essential for the effective operation of the visualization apparatus 600. The visualization apparatus comprises scanning catheter positioned within the housing 601 such that it scans the area of the abdominal aorta. The housing 601 is an extrusion of silicon, Teflon® or polymer or other material having similar properties. The scanning catheter extends through the minor guide wire portion 830 of housing 800. The scanning catheter creates an image of the repair that can be viewed on an external monitor, not shown.

The housing 800 also comprise transition portions 801 and 802 located on opposite ends of the penetration apparatus 700 to provide the repair apparatus 50000 with a smooth profile, as shown in FIG. 89. This improves the movement of the repair apparatus 50000 within the vessel 1000 and adjacent arteries.

### Fasteners

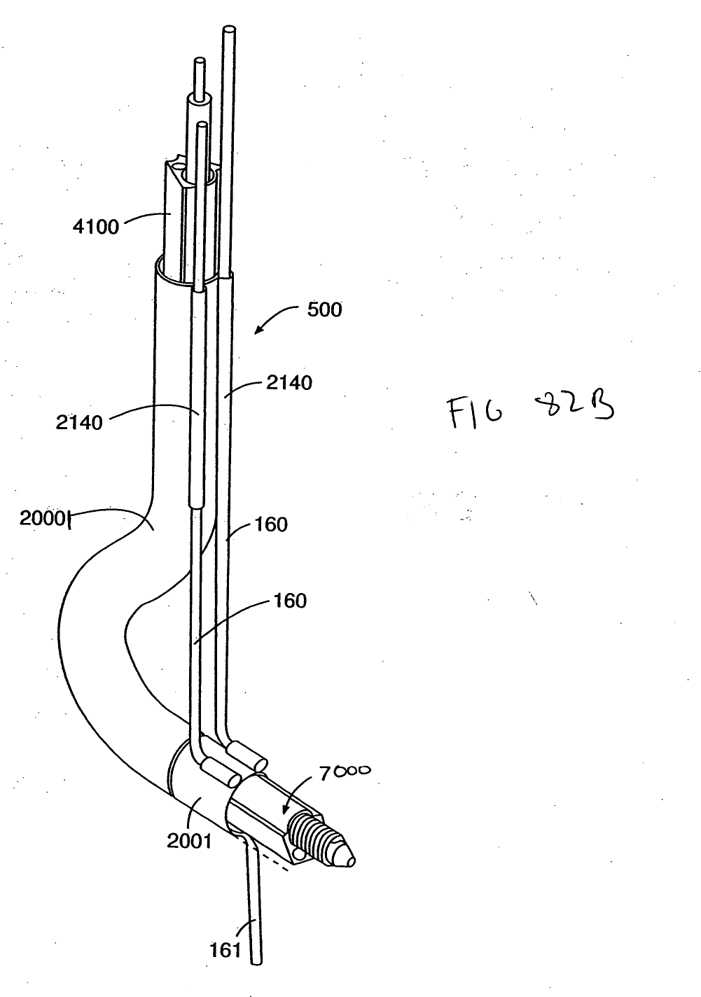

Reference will now be made in detail to preferred embodiments of a fastener device, as depicted in FIGS. 92-99, according to the present invention for securing the attachment device 20000 to the distal end of the vessel 1000. Although the fastener devices are described in connection with the repair of an aneurysm in a vessel, the use of the fastener devices in other surgical procedures as a replacement for sutures is contemplated to be within the scope of the present invention.

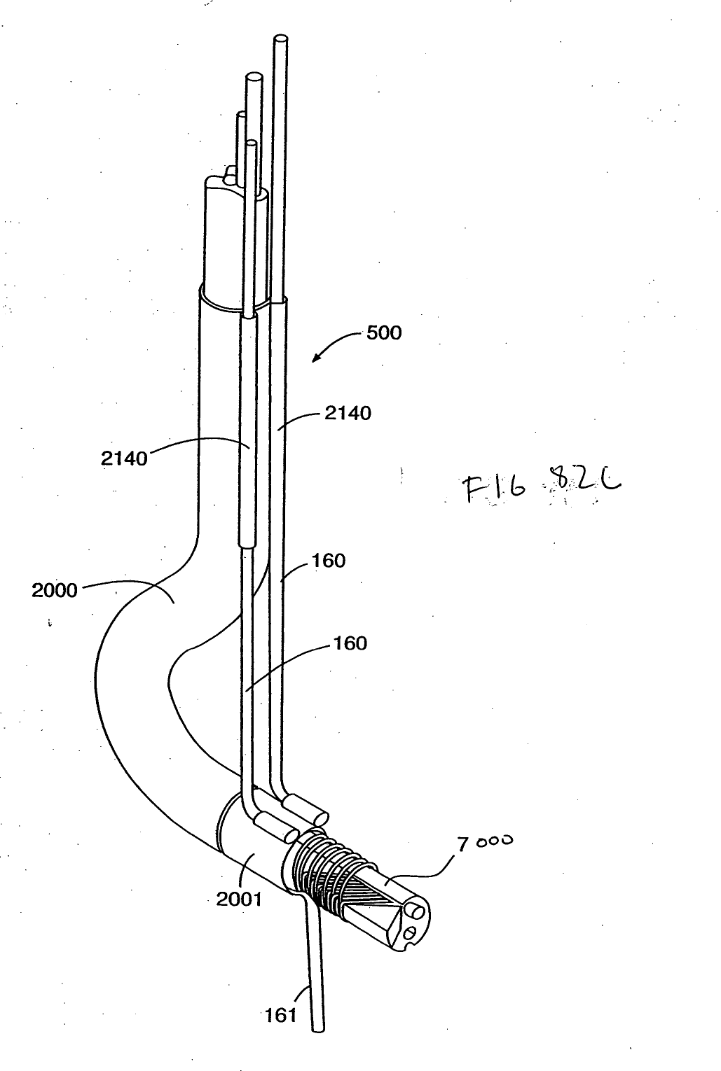

FIGS. 92 and 93 depict a fastener 510 according to an embodiment of the present invention. The fastener 510 comprises a pair of normally splayed fastening legs 512 and 513. The fastener 510 also comprises an anchoring portion 514. The fastener 510 is preferably formed from a wire-like material. The anchoring portion 514 may be formed from a coil of the wire-like material. The legs 512 and 513 are temporarily reoriented, as shown in FIG. 92, for storage on a fastener cartridge 460 and for enabling the attachment of the attachment device 20000 to the wall 2000. As the legs 512 and 513 are inserted through the attachment device 20000 and the wall 2000, the legs 512 and 513 return to a normal, as manufactured, splayed position, as shown in FIG. 93. When the fastener 510 is in a fastened position within the vessel, the anchoring portion 514 is positioned on one side of the attachment device 20000 and wall 2000 (intima/graft) adjacent the attachment device 20000. The splayed legs 512 and 513 are positioned on the opposite side of the attachment device 20000 and wall 2000 (adventia) adjacent the wall 2000. The anchoring portion 514 and splayed legs 512 and 513 apply compressive forces to the wall 2000 and the attachment device 20000 to securely fastening the attachment device 20000 to the vessel 1000.

The fastener 510 is preferably formed from a stainless steel, such that the legs 512 and 513 return to the splayed position to secure the attachment device 20000 to the wall 2000. It, however, will be apparent to those skilled in the art that various modifications and variations can be made in the construction and configuration of the present invention without departing from the scope or spirit of the invention. For example, the fastener 510 may be formed from other suitable materials including but not limited to superelastic titanium, or other procedure/performance-appropriate materials having similar properties including, but not limited to biocompatability, elasticity, and flexural strength. Thus, it is intended that the present invention cover the modifications and variations of the invention, provided they fall within the scope of the appended claims and their equivalents.

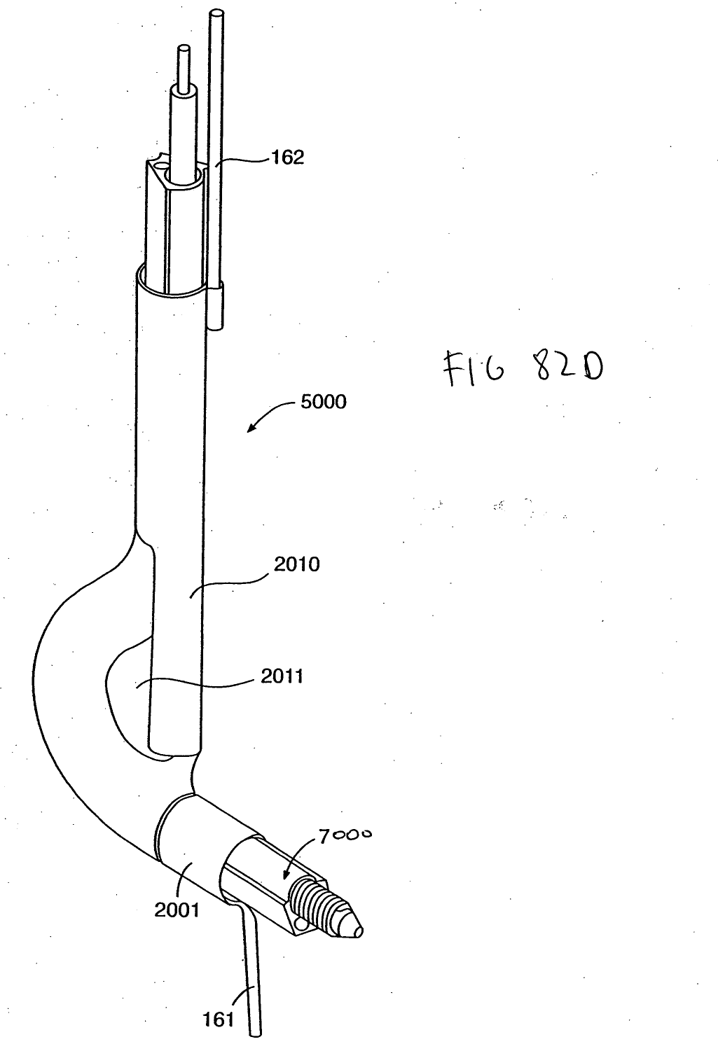

FIGS. 94 and 95 depict a fastener 520 according to an another embodiment of the present invention. The fastener 520 comprises a pair of normally splayed fastening legs 522 and 523. The fastener 520 also comprises an anchoring portion 524. The fastener 520 is also preferably formed from a wire-like material. The anchoring portion 524 may be formed from at least one coil of the wire-like material (i.e., a wound portion). The legs 522 and 523 are temporarily compressed, as shown in FIG. 94, for storage in a fastener cartridge 460 and for facilitating the attachment of the attachment device 20000 to the wall 2000. Similar to the embodiment described above in connection with FIGS. 92 and 93, as the legs 522 and 523 are inserted through the attachment device 20000 and the wall 2000, the legs 522 and 523 return to a normally splayed position, as shown in FIG. 94. When the fastener 520 is in a fastened position within the vessel, the anchoring portion 524 is positioned on one side of the attachment device 20000 and wall 2000 adjacent the attachment device 20000. The splayed legs 522 and 523 are positioned on another side of the attachment device 20000 and wall 2000 adjacent the wall 2000. The anchoring portion 524 and splayed legs 522 and 523 apply compressive forces to the wall 2000 and the attachment device 20000 to securely fastening the attachment device 20000 to the vessel 1000.

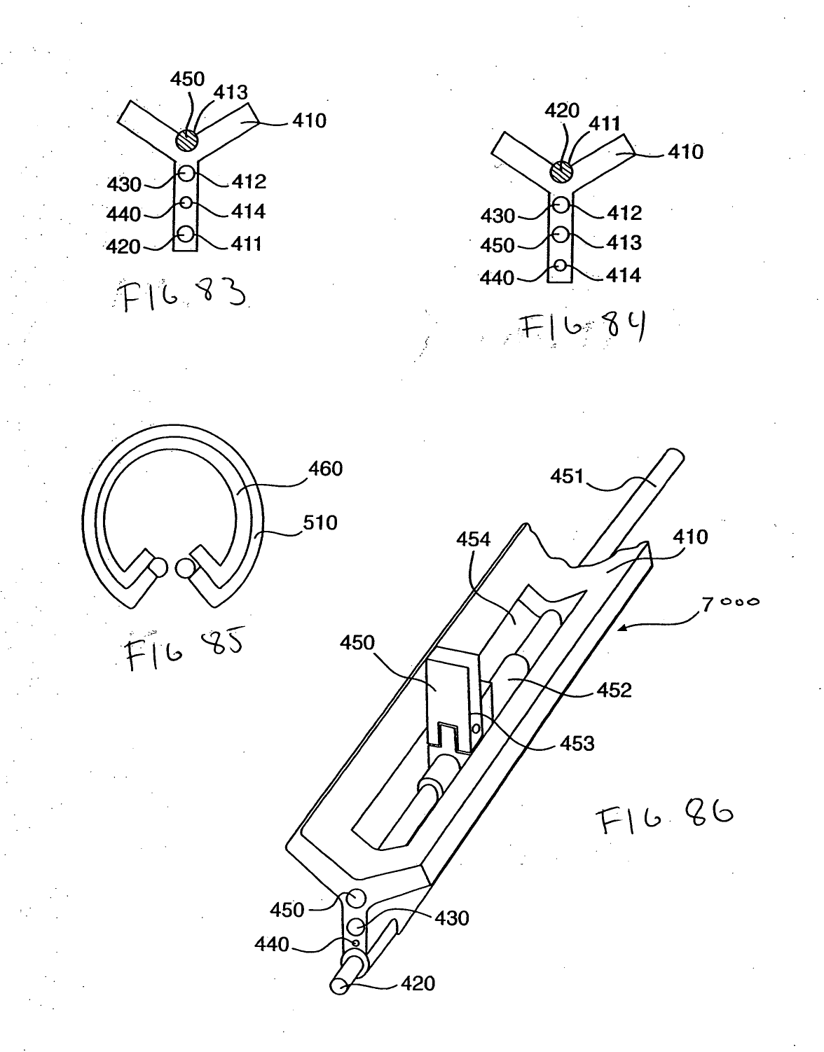

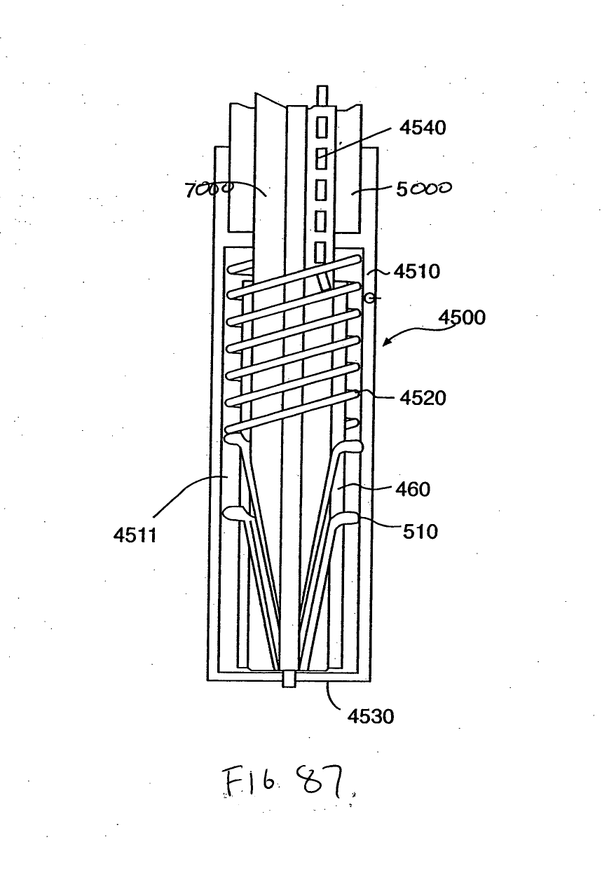

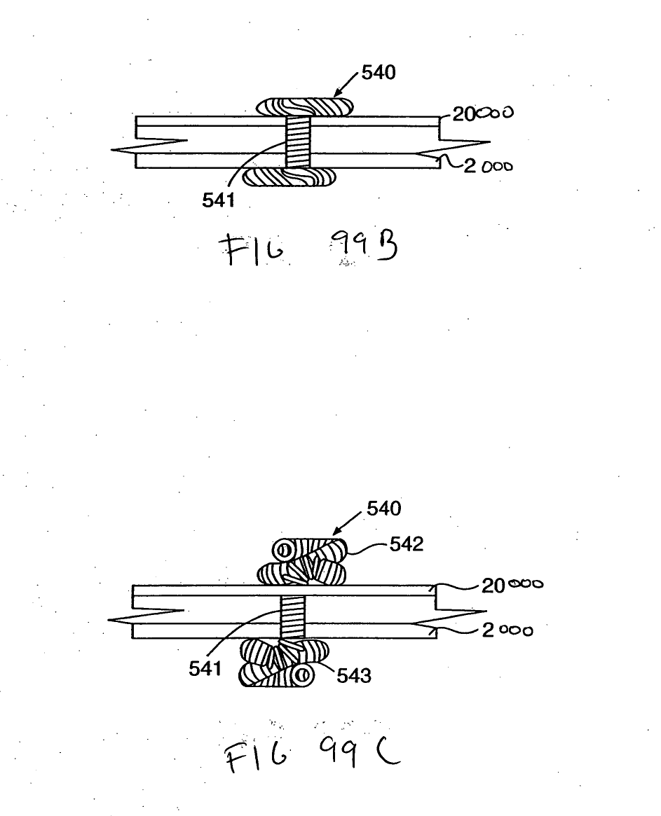

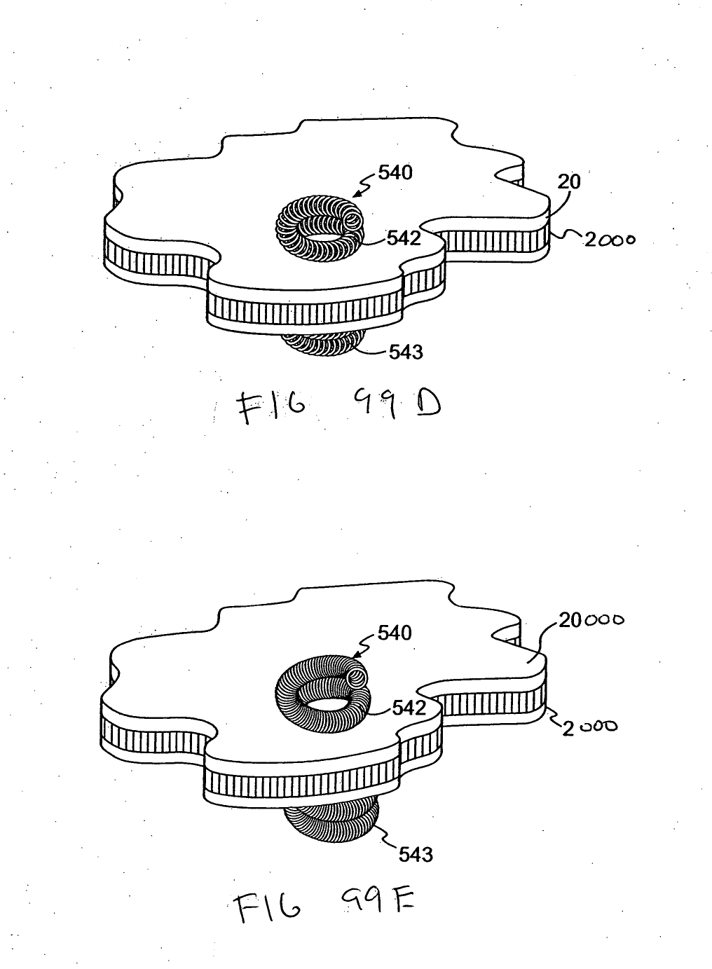

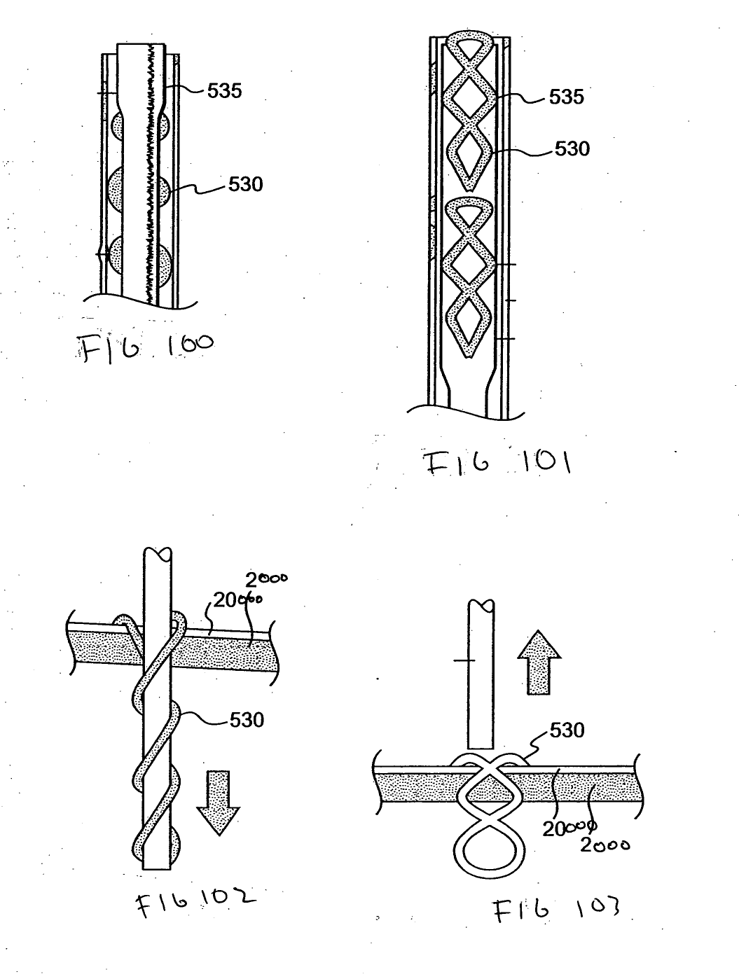

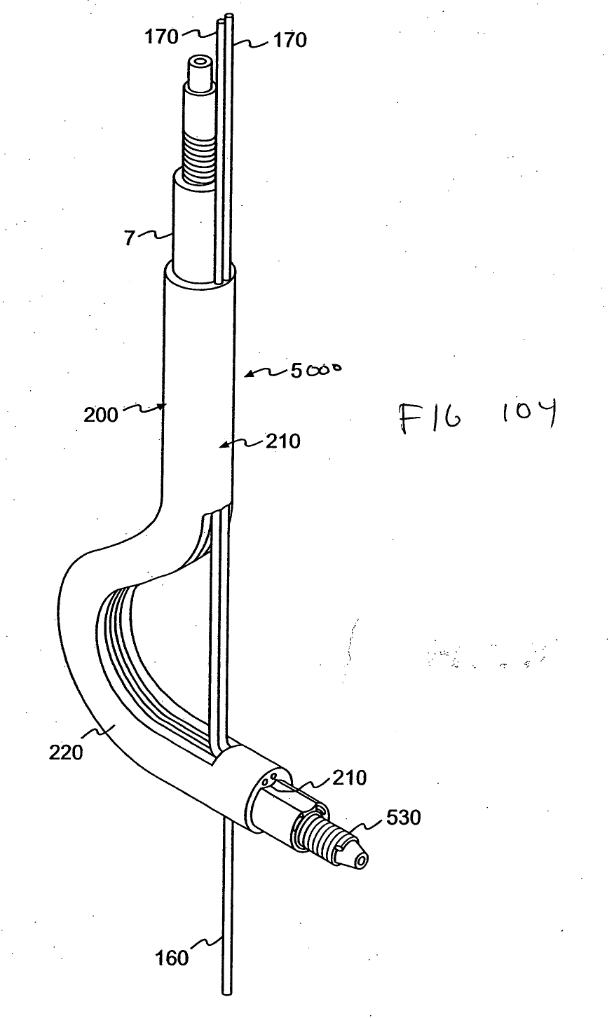

FIGS. 96 and 97 depict a fastener 530 according to another embodiment of the present invention. Fastener 530 is a spring type fastener, which may comprise a coil spring. The fastener 530 is also formed from a wire-like material. The fastener 530 comprises a plurality of coils, as shown in FIG. 96. The end portions 531 and 532 of the wire-like material are preferably located on the same end of the fastener 530, as shown in FIGS. 96, 97, and 101-103. Unlike fastener 510 and 520, the fastener 530 is temporarily elongated for storage in the fastener cartridge 535, as shown in FIGS. 100, 101, and 104. As the fastener 530 is inserted through the attachment device 20000 and wall 2000 using the insertion means 450 on the penetration device 7000, as shown in FIG. 102, the fastener 530 remains in an elongated position until the insertion means 450 is removed from the treatment specific hole 3000 created in the wall 2000 of the vessel 1000 and the attachment device 20000 formed by the penetration apparatus 7000. The fastener 530 then assumes a collapsed position, as shown in FIG. 97. When the fastener 530 is in a fastened position within the vessel 1000, the end portions 531 and 532 are positioned on one side of the attachment device 20000 and wall 2000 adjacent the attachment device 20000, as shown in FIG. 103. The remaining portion of the fastener 530 is positioned on another side of the attachment device 20000 and wall 2000 adjacent the wall 2000. The fastener 530 apply compressive forces to the wall 2000 and the attachment device 20000 to securely fastening the attachment device 20000 to the vessel 1000. Fastener 530 may be formed from a suitable shape memory alloy, stainless steel, titanium, a superelastic alloy, or any other procedure/performance-appropriate materials.

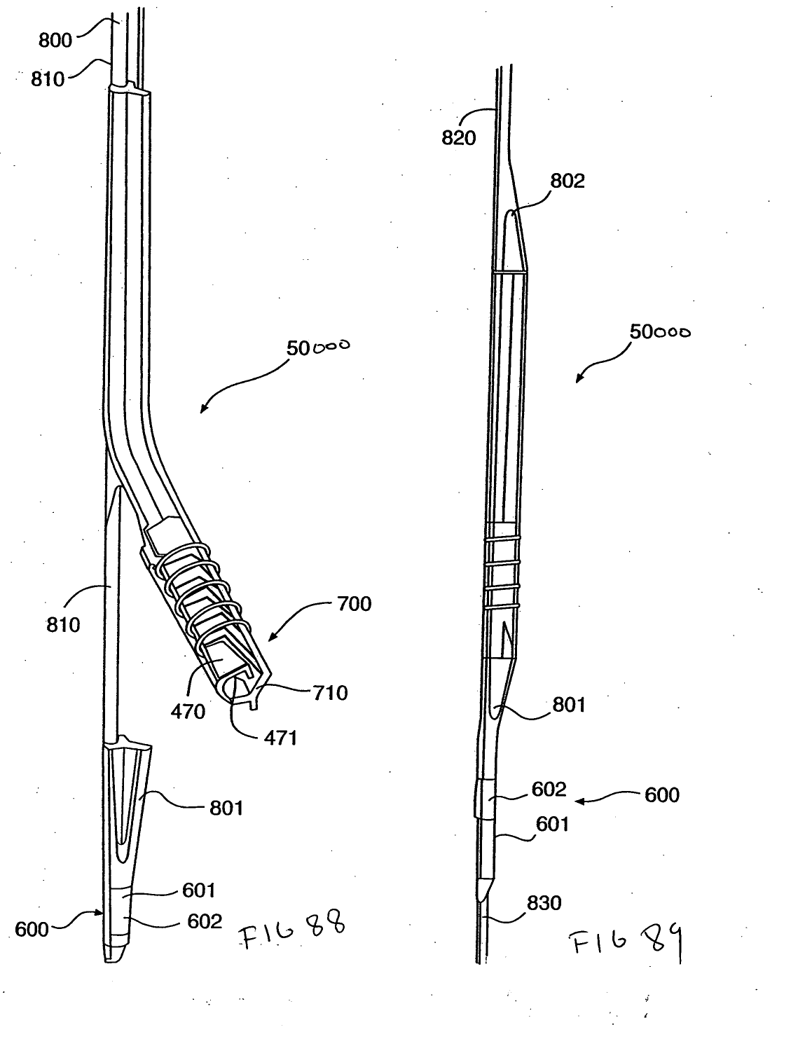

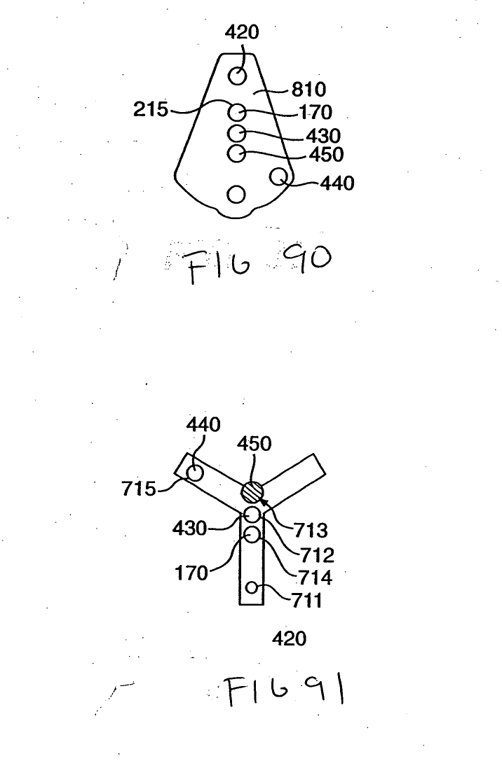

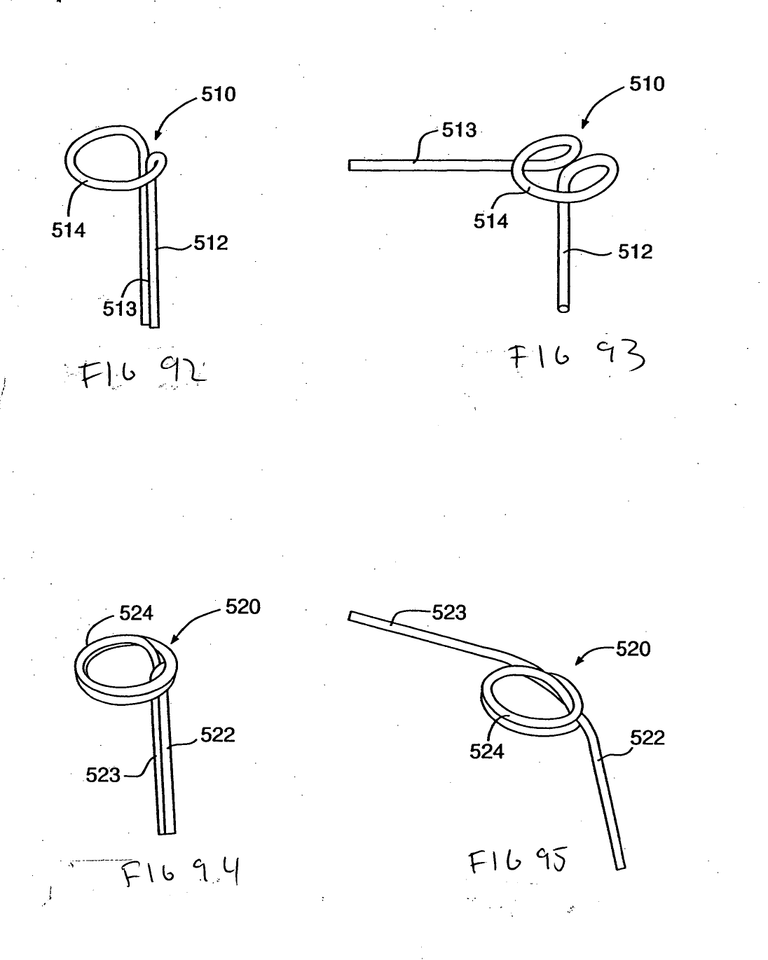

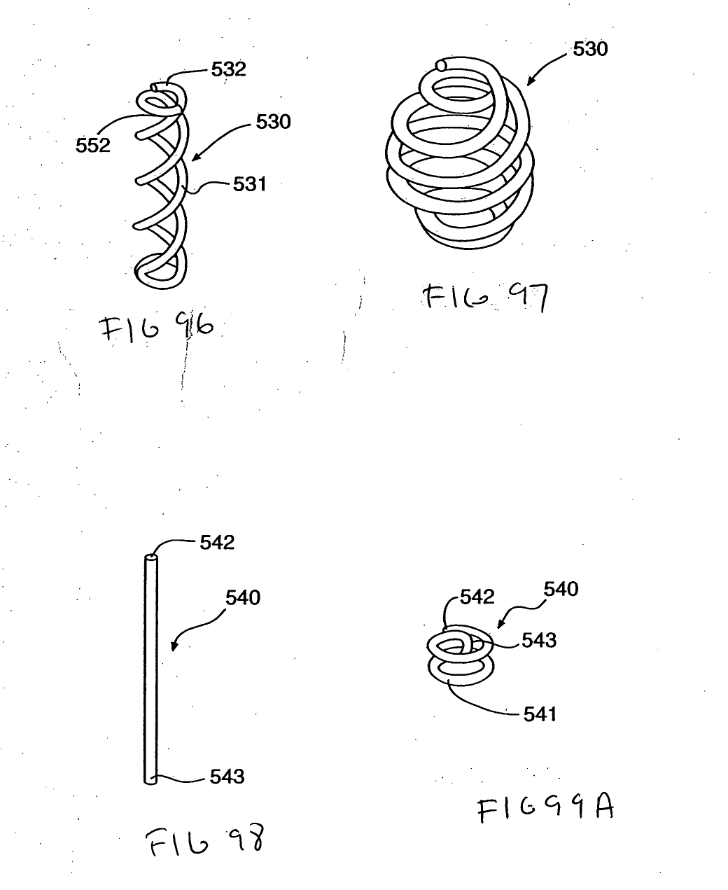

FIGS. 98, 99a, 99b, and 99c depict a fastener 540 according to another embodiment of the present invention. Fastener 540 is a coil spring type fastener. Fastener 540 comprises a mid-section 541, and semi-knotted end portions 542 and 543. The fastener 540 is also formed from a coil spring using materials, as described above. The fastener 540 is substantially linear, as shown in FIG. 98, for storage in a fastener cartridge, not shown. As the fastener 540 is inserted through the attachment device 20000 and wall 2000, the fastener 540 returns to its normally coiled position, as shown in FIG. 99a The fastener 540 applies compressive forces to the wall 2000 and the attachment device 20000 to securely fastening the attachment device 20000 to the vessel 1000 such that one semi-knotted end portion 542 is positioned adjacent the attachment device 20000 and the other semi-knotted end portion 543 is positioned adjacent the wall 2000 of the vessel 1000, as shown in FIGS. 99b and 99c. FIG. 99b depicts an axially wound fastener 540. FIG. 99d depicts the fastener 540 of FIG. 99b secured to the wall 2000. FIG. 99c depicts a radially wound fastner 540. FIG. 99e depicts the fastener 540 of FIG. 99c secured to the wall 2000.

It, however, will be apparent to those skilled in the art that various modifications and variations can be made in the construction and configuration of the present invention without departing from the scope or spirit of the invention. For example, the fastening means mentioned above, may be pop-rivet fasteners, screw-type fasteners, rapid hardening plastic extradites, and other suitable fasteners are contemplated to be within the scope of the present invention. Thus, it is intended that the present invention cover the modifications and variations of the invention, provided they fall within the scope of the appended claims and their equivalents.

### Introducer Sheath Devices

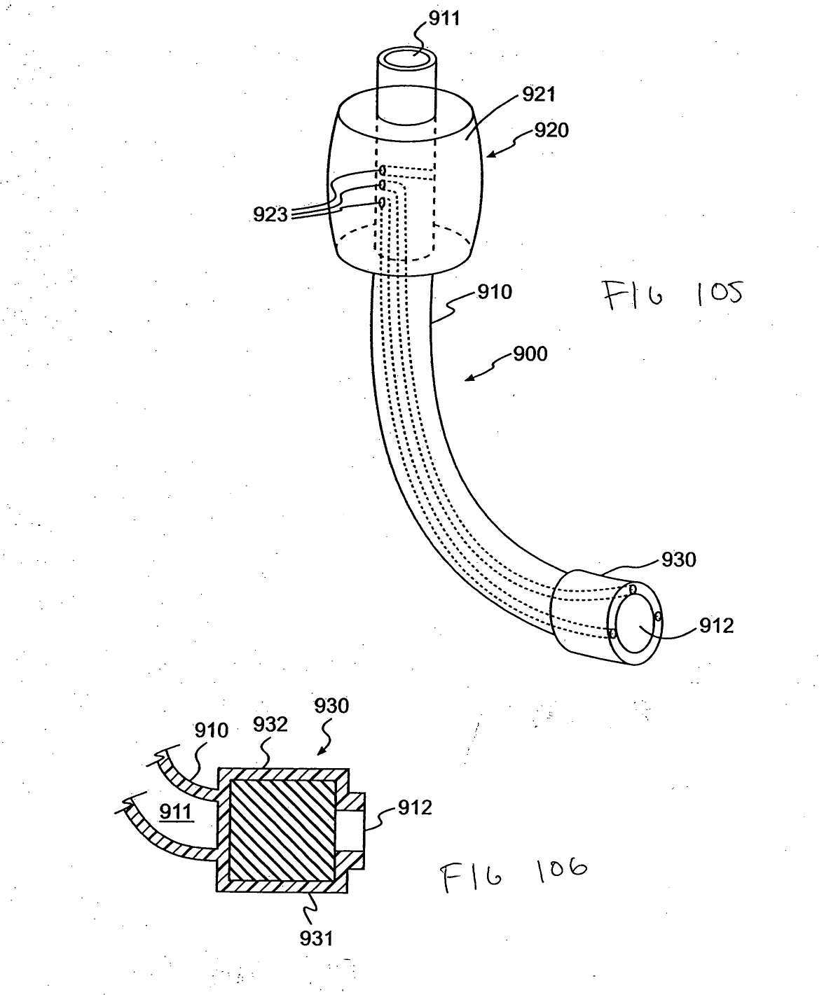

Reference will now be made to a preferred embodiment of an introducer sheath device according to the present invention of use in the repair of abdominal aneurysms, an example of which is illustrated in FIGS. 105 and 106. The introducer sheath devices create a protective passageway through the vessel through which the graft and repair devices are inserted. The introducer sheath devices protect the arteries from damage that may occur when the repair apparatus and other device are passed through the tortuous artery passageways during a surgical procedure.

Existing methods for repairing aneurysms utilize introducer sheath devices only in the femoral and common iliac arteries. Typically, guide wires extend from a femoral arteriotomy to an occlusion balloon placed within the proximal neck of the aorta at a point cephalad with respect to the abdominal aorta. Typically, others have gained access to the abdominal aorta via a femoral or common iliac arteriotomy into which is inserted an introducer sheath device of between 18-28 Fr. diameter. The size of these devices may cause damage to the vessels through which they pass.

By contrast, the inventors of the present invention contemplate the use of more than one unique introducer sheath device 900, as shown in FIG. 100. The sheaths 900 are introduced over a femoral/axillary guide wire. One introducer sheath device 900 extends from either an axillary incision or a brachial incision to the proximal neck of the vessel 1000. Another introducer sheath device 900 extends from a femoral incision to the distal neck of the vessel or common iliac/distal aorta transition. The introducer sheath devices according to the present invention that extend through the axillary vessel and through the femoral artery have similar constructions. However, the introducer sheath device that extends through the axillary artery has a smaller size in the range between 9-12 Fr. and is able to navigate the arteriotomy/distal aorta passageway without problem. The smaller size will permit access to the aorta via either the left brachial or axillary artery, both of which are significantly smaller than the femoral or common iliac arteries. This procedure, previously, beyond consideration, may now significantly benefit these vascular surgeries.

Each introducer sheath device 900 comprises a housing 910 having a hollow interior 911 that permits the passage of the tube graft and other repair apparatus through the introducer sheath device to the vessel 1000. The repair apparatus are introduced through the an opening 912 in the end portion of the housing 910. In a preferred embodiment, the housing 910 is a thin walled co-extrusion having an outer surface formed, for example, from silicon and an inner surface formed, for example, from Teflon®. Alternatively, the housing 910 may be formed of a suitable polymer having similar properties.

The introducer sheath device 900, also, comprises positioning assembly 920 for maintaining the sheath 900 in proper orientation within the vessel. In a preferred embodiment, the positioning assembly 920 comprises an inflatable cuff 921 located at one end of housing 910. The positioning assembly 920 further comprises an inflation device 922 for inflating the cuff 921. The inflation device 922 in a preferred embodiment comprises a plurality of passageways 923 formed within the wall of housing 910. A suitable fluid, such as saline, is supplied from an external source through the passageways 923 to fill the cuff 921. The passageways 923 terminate at inflatable cuff 921, as shown in FIG. 105.

Prior introducer sheath devices have not been able to control the loss of significant amounts of blood through the open end of the introducer sheath device that is positioned outside of the body. Others have attempted to prevent this blood loss through the use of complex clamping systems. The present invention provide a unique seal arrangement to prevent significant blood loss. A seal 930 located at one end of the housing 910 adjacent opening 912 will prevent significant blood loss. The seal 930 comprises an expanded housing assembly 931. A self-sealing gel-like material 932 is located within the expanded housing assembly 931. The material 932 permits the insertion of the repair apparatus through the material 932, which forms a seal around the repair apparatus. As a the repair apparatus is removed from the introducer sheath device 900, the material 932 forms a seal behind the repair apparatus as it is removed through opening 912.

It, however, will be apparent to those skilled in the art that various modifications and variations can be made in the construction and configuration of the present invention without departing from the scope or spirit of the invention. It is intended that the present invention cover the modifications and variations of the invention, provided they fall within the scope of the appended claims and their equivalents.

### Method of Repairing an Aneurysm

Reference will now be made in detail to a preferred embodiment of the method of repairing an aneurysm utilizing the above described components according to the present invention.

### Intravascular Endoscopy (IVE) Based Repair Method

The IntraVascular Endoscopy (IVE) based repair method will be described in connection with the use of a proximal graft assembly 10000 and distal graft assembly 2000. Introducer sheath devices 900 are placed by femoral arteriotomy in both common iliacs under radiological guidance such that the positioning assembly 920 is positioned at the common iliac/aortic bifurcation transition. A guide wire is fed from one femoral incision to the other, also under guidance. A distal graft assembly 2000 is fed over the guide wire until the attachment cuff 2100 appears directly above the carina at the bifurcation, as shown in FIG. 68A.

A second guide wire 160 is now fed under radiological guidance between one femoral incision and the left axillary incision. Another introducer sheath device 900 is fed from the axillary until the positioning assembly 920 reaches the infrarenal aorta at which time it is inflated. The repair apparatus 5000 is then fed through the introducer sheath device 900 over guide wire 160 from either the femoral or axillary access to the midpoint of the aortic aneurysm. The visualization apparatus 6000 is then fed through the hollow interior 211 of housing 200 to the area of the wall 2000 to which the attachment cuff 22000 is to be attached. The guide wires 170 are then manipulated to adjust the orientation of the visualization apparatus 6000 to permit viewing of the wall 2000 as described above, such that an image appears on the monitor. An image of the wall 2000 appears on the monitor. The visualization apparatus 6000 is then removed and the penetration apparatus 7000 is then inserted through the hollow interior 211. The guide wires 170, as described above, permit the penetration apparatus to be positioned in the same position as the visualization apparatus 6000. The tracking means 440 pinpoints the location of the penetration means 420 with respect to the wall 2000 and attachment cuff 22000 as viewed on the monitor. The primary and secondary penetration means 420 and 430 are then operated to form treatment specific holes within the cuff 22000 and wall 2000, as described above. The primary penetration means 420 is then retracted and a fastener is then inserted within the treatment specific hole using the insertion means 450. The location of the penetration apparatus 7000 is then adjusted to repeat the process over the area previously viewed by the visualization apparatus 6000. The penetration apparatus 7000 is then removed and the visualization apparatus 6000 is reinserted. The viewing and fastening process is alternately repeated until the attachment cuff 22000 is firmly attached to the wall 2000.

The repair apparatus 5000 is removed once the attachment cuff 22000 is secured to the wall 2000. The proximal graft assembly 10000 is then inserted in an inverted manner through the femoral arteriotomy over the guide wire 160 to the position shown in FIG. 68A. The repair apparatus 5000 is then fed through the introducer sheath device 900 over the guide wire 160 from either a femoral or axillary access to a position adjacent the attachment cuff 12000. The visualization apparatus 6000 and the penetration apparatus 7000 are then alternately inserted in the manner described above to secure the attachment cuff 12000 to the wall 2000. In the event that a standard graft 3000 is used, the inverted graft 3000 is secured directly to the wall 2000 in a similar manner. Alternatively, a self-expanding stent 30000 may be used in combination with fasteners to secure the graft 3000 to the wall 2000. The repair apparatus 5000 is then removed.

Once the proximal graft assembly 10000 or 3000 are secured in place, the first guide wire is removed and the graft 3000 or 10000 is invaginated. The tubular legs 1100 are then inserted into the attachment tubes 22000. Self-expanding stents 30000 are then used to secure the attachment tubes 22000 and tubular legs 1100 firmly together. The guide wire 160 is then removed. The positioning assemblies 920 are deflated and the introducer sheath device 900 are removed from the femoral and axillary arteries. The incisions are then closed completing the repair process. The outlined procedure according to the present invention is far less intrusive than current known techniques. As a result, a patient's recovery period should decrease.

### IntraVascular Ultrasound Repair Method

The intravascular ultrasound repair method will be described in connection with the use of a proximal graft assembly 10000 and distal graft assembly 2000. Introducer sheath device 900 are placed by femoral arteriotomy in both common iliacs under radiological guidance such that the positioning assembly 920, as described above in connection with the IntraVascular Endoscopy (IVE) based repair method. A distal graft assembly 2000 is fed over a guide wire, as described above, until the attachment cuff 2100 appears directly above the carina at the bifurcation, as shown in FIG. 68A.

A second guide wire 160 is now fed under guidance between one femoral incision and the left axillary incision. Another introducer sheath device 900 is fed from the axillary until the positioning assembly 920 reaches the infrarenal aorta at which time it is inflated. The repair apparatus 50000 is then fed through the introducer sheath device 900 over guide wire 160 to the midpoint of the aortic aneurysm. The visualization apparatus 600 is then positioned adjacent the area of the wall 2000 to which the attachment cuff 22000 is to be attached. The scanning catheter is drawn caudad providing images of the distal aortal common iliac transition on an external monitor. The repair apparatus 50000 is then oriented such that the penetration apparatus 700 is adjacent the area where the attachment cuff 2100 is to be attached to the wall 2000.

The primary and secondary penetration means 420 and 430 are then operated to form treatment specific holes within the cuff 22000 and wall 2000, as described above. The primary penetration means 420 is then retracted and a fastener is then inserted within the treatment specific hole using the insertion means 450. The location of the penetration apparatus 700 is then adjusted to repeat the process over the area previously viewed by the visualization apparatus 600. The repair apparatus 50000 is then oriented such that the visualization apparatus 600 may scan another portion of the wall 2000. The viewing and mounting process is alternately repeated until the attachment cuff 22000 is firmly attached to the wall 2000.

The repair apparatus 50000 is removed once the attachment cuff 22000 is secured to the wall 2000. The proximal tube graft assembly 10000 is then inserted in an inverted manner over the guide wire 160 to the position shown in FIG. 68A. The repair apparatus 50000 is then inserted through a femoral incision over the guide wire 160 to a position adjacent the attachment cuff 12000. The visualization apparatus 600 and the penetration apparatus 700 are then alternately operated in the manner described above to secure the attachment cuff 12000 to the wall 2000.

Once the proximal graft assembly 10000 is secured in place, the first guide wire and the repair apparatus 50000 are removed and the proximal graft assembly 10000 is invaginated. The tubular legs 11 are then inserted into the attachment tubes 22000 or vice verse. Self-expanding stents 30 are then used to secure the attachment tube 22000 and tubular legs 11 firmly together. The guide wire 160 is then removed. The positioning assemblies 920 are deflated and the introducer sheath devices 900 are removed from the femoral and axillary arteries. The incisions are then closed completing the repair process.

While this invention has been described in conjunction with specific embodiments thereof, it is evident that many alternatives, modifications and variations will be apparent to those skilled in the art. Accordingly, the preferred embodiments of the invention as set forth herein are intended to be illustrative, not limiting. Various changes may be made without departing from the spirit and scope of the invention as defined in the following claims.

What is claimed is:

1. An insertion apparatus for deploying a surgical component to a site in a vessel comprising:
a first attachment device releasably attaching the surgical component to the insertion apparatus, and
a second attachment device housing at least one flexible surgical fastener adapted to attach the surgical component to the surgical site.

2. The insertion apparatus of claim 1, wherein the surgical component is a graft.

3. The insertion apparatus of claim 1, wherein the first attachment device supplies an electric current to release the surgical component from the insertion apparatus.

4. The insertion apparatus of claim 1, wherein the first attachment device supplies heat to release the surgical component from the insertion apparatus.

5. The insertion apparatus of claim 1, wherein the surgical component comprises at least one distinctive marker.

6. The insertion apparatus of claim 1, wherein the first attachment device comprises at least one attachment wire and a support wire.

7. An insertion apparatus for deploying a surgical component to a site in a vessel comprising:
at least one attachment wire with a first end and a second end;
a support wire;
wherein the first end of the at least one attachment wire is releaseably connected to the surgical component and the second end is connected to the support wire;
wherein the attachment wire and support wire positions the surgical component at a site in the vessel, and an attachment device housing at least one flexible surgical fastener adapted to attach the surgical component to the surgical site.

8. The insertion apparatus of claim 7, further comprising a fastener delivery catheter.

9. The insertion apparatus of claim 7, wherein the surgical component is a graft.

10. The insertion apparatus of claim 7, further comprising an electric current supplied to the attachment wire for releasing the surgical component.

11. The insertion apparatus of claim 7, wherein the surgical component comprises at least one distinctive marker.

12. The insertion apparatus of claim 7, wherein the insertion apparatus further comprises a short sheath.

13. The insertion apparatus of claim 12, wherein the short sheath comprises a flare.

14. The insertion apparatus of claim 7, wherein the surgical component corresponds to the site in the vessel with an appropriate diameter and length.

15. The insertion apparatus of claim 7, further comprising an introducer sheath.

16. The insertion apparatus of claim 15, wherein the introducer sheath comprises a heemostatic valve and a dilator.

17. An insertion apparatus for deploying a surgical component to a site in a vessel comprising:
an attachment hoop releasably connected to the surgical component;
at least one attachment wire with a first end and a second end;
a support wire;
wherein the first end of the at least one attachment wire is connected to the attachment hoop and the second end of the at least one attachment wire is connected to the support wire;
wherein the insertion apparatus positions the surgical component in the vessel; and
an attachment device housing at least one flexible surgical fastener adapted to attach the surgical component to the surgical site.

18. The insertion apparatus of claim 17, further comprising a fastener delivery catheter.

19. The insertion apparatus of claim 17, wherein the surgical component is a graft.

20. The insertion apparatus of claim 17, further comprising an electric current supplied to the attachment hoop for releasing the surgical component.
